SEQUENCE LISTING (49 sequences)

SEQ ID NO: 1 - DNA, 17 bp, synthetic construct
  source 1..17: mol_type = other DNA
agtggtaccgtctgcta

SEQ ID NO: 2 - DNA, 17 bp, synthetic construct
  source 1..17: mol_type = other DNA
cttctactgctcacagg

SEQ ID NO: 3 - DNA, 17 bp, synthetic construct
  source 1..17: mol_type = other DNA
cgggtgcttgccatctt

SEQ ID NO: 4 - DNA, 17 bp, synthetic construct
  source 1..17: mol_type = other DNA
tgccattctgaagccgg

SEQ ID NO: 5 - DNA, 17 bp, Homo sapiens
  source 1..17: mol_type = genomic DNA
cgggtgcttgccatctt

SEQ ID NO: 6 - DNA, 17 bp, Homo sapiens
  source 1..17: mol_type = genomic DNA
gtgcttgccatcttcat

SEQ ID NO: 7 - DNA, 17 bp, Homo sapiens
  source 1..17: mol_type = genomic DNA
cttgccatcttcatctg

SEQ ID NO: 8 - DNA, 17 bp, Homo sapiens
  source 1..17: mol_type = genomic DNA
ccatcttcatctgcagc

SEQ ID NO: 9 - DNA, 17 bp, Homo sapiens
  source 1..17: mol_type = genomic DNA
cagcgggtgcttgccat

SEQ ID NO: 10 - DNA, 17 bp, Homo sapiens
  source 1..17: mol_type = genomic DNA
ttctgccattctgaagc

SEQ ID NO: 11 - DNA, 17 bp, Homo sapiens
  source 1..17: mol_type = genomic DNA
tgccattctgaagccgg

SEQ ID NO: 12 - DNA, 17 bp, Homo sapiens
  source 1..17: mol_type = genomic DNA
ccattctgaagccgggt

SEQ ID NO: 13 - DNA, 17 bp, Homo sapiens
  source 1..17: mol_type = genomic DNA
atcatcttctgccattc

SEQ ID NO: 14 - DNA, 17 bp, Homo sapiens
  source 1..17: mol_type = genomic DNA
atcttctgccattctga

SEQ ID NO: 15 - DNA, 17 bp, Mus musculus
  source 1..17: mol_type = genomic DNA
agtggtaccgtctgcta

SEQ ID NO: 16 - DNA, 17 bp, Mus musculus
  source 1..17: mol_type = genomic DNA
cttctactgctcacagg

SEQ ID NO: 17 - protein, 4 aa, synthetic construct
AEEA

SEQ ID NO: 18 - protein, 5 aa, synthetic construct
HHHHH

SEQ ID NO: 19 - DNA, 15 bp, synthetic construct
  source 1..15: mol_type = other DNA
taaagttcgtttaga

SEQ ID NO: 20 - DNA, 15 bp, synthetic construct
  source 1..15: mol_type = other DNA
gctctccatcttacc

SEQ ID NO: 21 - DNA, 15 bp, synthetic construct
  source 1..15: mol_type = other DNA
acaccgcaaacccgt

SEQ ID NO: 22 - DNA, 15 bp, synthetic construct
  source 1..15: mol_type = other DNA
cgggctgcacttaca

SEQ ID NO: 23 - DNA, 15 bp, synthetic construct
  source 1..15: mol_type = other DNA
tactagtgcctgtgc

SEQ ID NO: 24 - DNA, 15 bp, synthetic construct
  source 1..15: mol_type = other DNA
gtatacgacatcagt

SEQ ID NO: 25 - protein, 7 aa, simian virus 40
PKKKRKV

SEQ ID NO: 26 - protein, 20 aa, unidentified
AVKRPAATKKAGQAKKKKLD

SEQ ID NO: 27 - protein, 9 aa, Homo sapiens
PAAKRVKLD

SEQ ID NO: 28 - protein, 25 aa, Caenorhabditis elegans
MSRRRKANPTKLSENAKKLAKEVEN

SEQ ID NO: 29 - protein, 9 aa, Escherichia coli
KLKIKRPVK

SEQ ID NO: 30 - protein, 9 aa, Homo sapiens
ETFSDLWKL

SEQ ID NO: 31 - protein, 9 aa, Homo sapiens
DDIEQWFTE

SEQ ID NO: 32 - protein, 9 aa, Homo sapiens
SDIMDFVLK

SEQ ID NO: 33 - protein, 9 aa, Homo sapiens
DLLDFSMMF

SEQ ID NO: 34 - protein, 9 aa, Saccharomyces cerevisiae
ETLDFSLVT

SEQ ID NO: 35 - protein, 9 aa, Homo sapiens
RKILNDLSS

SEQ ID NO: 36 - protein, 9 aa, Homo sapiens
EAILAELKK

SEQ ID NO: 37 - protein, 9 aa, Homo sapiens
DDVVQYLNS

SEQ ID NO: 38 - protein, 9 aa, Saccharomyces cerevisiae
DDVYNYLFD

SEQ ID NO: 39 - protein, 9 aa, Saccharomyces cerevisiae
DLFDYDFLV

SEQ ID NO: 40 - protein, 9 aa, Homo sapiens
DFFDYDLLF

SEQ ID NO: 41 - protein, 9 aa, Saccharomyces cerevisiae
EDLYSILWS

SEQ ID NO: 42 - protein, 9 aa, Saccharomyces cerevisiae
TDLYHTLWN

SEQ ID NO: 43 - protein, 22 aa, synthetic construct
AQQAQQQQQNQAQQAQQQQQNQ

SEQ ID NO: 44 - protein, 15 aa, synthetic construct
PPPDLGPPPDLGPPP

SEQ ID NO: 45 - protein, 19 aa, synthetic construct
KSHAHAQKRIRRRLLIILL

SEQ ID NO: 46 - DNA, 12 bp, synthetic construct
  source 1..12: mol_type = other DNA
atcaccaagtag

SEQ ID NO: 47 - DNA, 12 bp, synthetic construct
  source 1..12: mol_type = other DNA
tgtgttacgcta

SEQ ID NO: 48 - DNA, 12 bp, synthetic construct
  source 1..12: mol_type = other DNA
gtgacatacatt

SEQ ID NO: 49 - protein, 10 aa, synthetic construct
KFFKFFKFFK

(19) United States
(12) Patent Application Publication
(10) Pub. No.: US 2024/0150408 A1
(43) Pub. Date: May 9, 2024

(54) METHODS AND SYSTEMS FOR TARGETING AUTOIMMUNE AND INFLAMMATORY PATHWAYS USING NANOLIGOMERS

(71) Applicant: Sachi Bioworks Inc., Louisville, CO (US)

(72) Inventor: Prashant Nagpal, Lafayette, CO (US)

(73) Assignee: Sachi Bioworks Inc., Louisville, CO (US)

(21) Appl. No.: 18/376,197

(22) Filed: Oct. 3, 2023

Related U.S. Application Data
(63) Continuation-in-part of application No. 18/137,101, filed on Apr. 20, 2023.
(60) Provisional application No. 63/409,294, filed on Sep. 23, 2022.
(60) Provisional application No. 63/335,485, filed on Apr. 27, 2022.
(60) Provisional application No. 63/344,152, filed on May 20, 2022.
(60) Provisional application No. 63/390,909, filed on Jul. 20, 2022.

(51) Int. Cl. C07K 14/00; B82Y 5/00

(52) U.S. Cl. CPC C07K 14/003; B82Y 5/00; C07K 2319/09

(57) ABSTRACT

Compositions and methods involving nanoligomers are disclosed herein. Nanoligomers may include a targeting sequence and a nanostructure. A targeting sequence may include a polynucleotide binding domain and a transcription activation domain. A nanostructure may include a nanoparticle and a cell uptake domain.

### CROSS-REFERENCE TO RELATED APPLICATIONS

This application is a continuation-in-part of Non-provisional application Ser. No. 18/137,101 filed on Apr. 20, 2023 and entitled “METHODS AND SYSTEM FOR TARGETING AUTOIMMUNE AND INFLAMMATORY PATHWAYS USING NANOLIGOMERS,” which claims the benefit of priority of U.S. Provisional PATENT APPLICATION Ser. No. 63/409,294, filed on Sep. 23, 2022, and titled “METHODS AND SYSTEMS FOR TARGETING NEUROINFLAMMATORY PATHWAYS USING NANOLIGOMERS,” and U.S. Provisional Patent Application Ser. No. 63/335,485, filed on Apr. 27, 2022, and titled “METHODS AND SYSTEMS FOR REVERSING RADIATION-INDUCED IMMUNOSUPPRESSION,” and U.S. Provisional Patent Application Ser. No. 63/344,152, filed on May 20, 2022, and titled “OLIGOMER FOR MITIGATION OF NEUROINFLAMMATORY DISEASE, AND A METHOD FOR MANUFACTURING THE SAME,” and U.S. Provisional Patent Application Ser. No. 63/390,909, filed on Jul. 20, 2022 and titled “METHOD FOR MITIGATING A VIRAL AGENT INFECTION IN A SUBJECT AND SYSTEM FOR BIODISTRIBUTING A NANOLIGOMER IN A SUBJECT,” each of which is incorporated by reference herein in its entirety.

### FIELD OF THE INVENTION

The present invention generally relates to the field of health and biology. In particular, the present invention is directed to methods and systems for targeting autoimmune and inflammatory pathways using Nanoligomers.

### BACKGROUND

Neuroinflammation is a key factor in the development of neurodegenerative diseases, including prion disease. Pathogenesis includes accumulation of misfolded proteins, synaptic dysfunction, and cognitive/behavioral deficits followed by irreversible neuronal death, with no effective treatments to halt or slow the progression.

Even beyond neuroinflammation, same pathways are implicated in a range of autoimmune diseases such as Ulcerative Colitis, Crohn's Disease, Psoriasis, and many others. Unintended activation of human immune system which attacks healthy cells and organs represents one of the biggest unmet clinical need and pharmaceutical market.

### SUMMARY OF THE DISCLOSURE

In an aspect, a nanoligomer includes a targeting sequence, wherein the targeting sequence comprises a polynucleotide binding domain and a transcription activation domain; and a nanostructure, wherein the nanostructure comprises a cell uptake domain.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is an illustration of an exemplary embodiment of a nanoligomer;

FIG. 2 shows exemplary cognition and behavioral deficits are protected by nanoligomers treated by intraperitoneal route in accordance with aspects of the disclosure;

FIG. 3 shows exemplary microglia and astrocytic inflammation are significantly reduced using nanoligomers in prion diseased mice in accordance with aspects of the disclosure;

FIG. 4 shows exemplary prion induced spongiotic change and neuronal loss that is significantly decreased with Nanoligomer SB_NI_112 treatment in accordance with aspects of the disclosure;

FIG. 5 is a block diagram of an exemplary embodiment of a machine-learning module that may perform one or more machine-learning processes in accordance with aspects of the invention;

FIG. 6 shows additional exemplary cognition and behavioral deficits are protected by nanoligomers treated by intraperitoneal route in accordance with aspects of the disclosure;

FIG. 7 shows exemplary microglia and astrocytic inflammation are significantly reduced using nanoligomers in prion diseased mice in accordance with aspects of the disclosure;

FIG. 8 shows exemplary prion induced spongiotic change and neuronal loss that is significantly decreased with Nanoligomer SB_NI_112 treatment in accordance with aspects of the disclosure; and

FIG. 9 shows exemplary prion induced spongiotic change and neuronal loss that is significantly decreased with Nanoligomer SB_NI_112 treatment in accordance with aspects of the disclosure;

FIG. 10 shows results of a study examining SB_NI_112 availability within different regions of the brain;

FIG. 11A-F shows results of an animal model of autoimmune disease with or without SB_NI_112;

FIG. 12A-B depicts flow cytometry results and an experiment design for determining Treg populations;

FIG. 13 is a diagram showing how brain penetrant nanoligomers targeting NF-KB and NLRP3 may inhibit up and downstream causes of neuroinflammation in aging/AD;

FIG. 14 shows H&E stained first pass and clearance organs with no accumulation or immunogenic response using 500 mg/kg dosing of NF-KB/NLRP3 downregulating SB_NI_112 treatment;

FIG. 15A-D show data on the impacts on cytokines and photos of inflammation from a mouse model with or without SB_NI_112;

FIG. 16A-B shows data on novel object recognition and elevated maze test performance of mice with or without SB_NI_112;

FIG. 17A-B shows a heatmap of top age-related RNA-seq changes and representative immunohistochemistry staining for H&E stained liver of mice with or without SB_NI_112;

FIG. 18A-B shows data on cytokine levels, and novel object recognition and elevated maze test performance of rTG4510 mice with or without SB_NI_112;

FIG. 19A-B shows representative immunohistochemistry staining for T-217 stain for phosphorylated tau in old mice study; and rTG4510 mice;

FIG. 20A-B shows disease activity index and cytokine data from a DSS-induced colitis mouse study;

FIG. 21 shows design of nanoligomer platform for targeting BGCs in gut microbes;

FIG. 22 shows selection criteria used to identify nanoligomers;

FIG. 23A-B presents data on nanoligomer targeting of specific species;

FIG. 24A-D shows data on a relationship between nanoligomers targeting key immunomodulatory metabolites and host immune response;

FIG. 25A-B shows a gut-immune response in vitro model for cell lysate and cell supernatant using IL-18 expression by donor-derived PBMCs;

FIG. 26A-B shows data on changes in metabolite profile of Eubacterium rectale in response to nanoligomer treatment;

FIG. 27A-B shows nanoligomer PK/PD safety and toxicology study results;

FIG. 28A-F provides a design of a dextran sodium sulfate (DSS) mouse model and results of the study;

FIG. 29A-E provides results of a 3 week DSS study for mice given or not given SB_NI_112;

FIG. 30A-D provides results with and without SB_NI_112 in a genetic TNF^{ΔARE} model;

FIG. 31A-D provides data on a safety and PK/PD study in a canine model; and

FIG. 32 is a block diagram of a computing system that can be used to implement any one or more of the methodologies disclosed herein and any one or more portions thereof in accordance with aspects of the invention.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Incorporated herein by reference is a sequence listing XML file entitled, “1186-006USC1 sequence listing 3.xml,” created on January 11, 2024, and including a size of 42.2 KB (43,626 bytes).

In some embodiments, a nanoligomer described herein is capable of regulating the NLRP3 inflammasome. The NLRP3 inflammasome is a component of the innate immune system that, among other functions, modifies production and secretion of proinflammatory cytokines such as IL-1B and IL-18. Activation of the NLRP3 inflammasome is associated with inflammatory diseases such as Alzheimer's disease. In some embodiments, a nanoligomer is capable of downregulating NLRP3 inflammasome activity. In some embodiments, a nanoligomer is capable of downregulating expression of a component of the NLRP3 inflammasome. In some embodiments, a nanoligomer is capable of downregulating NF-κβ activity. In some embodiments, a composition comprises a nanoligomer capable of downregulating NLRP3 inflammasome activity and a nanoligomer capable of downregulating NF-κβ activity. In some embodiments, a subject in need thereof is treated by administration of a composition comprising one or more nanoligomers.

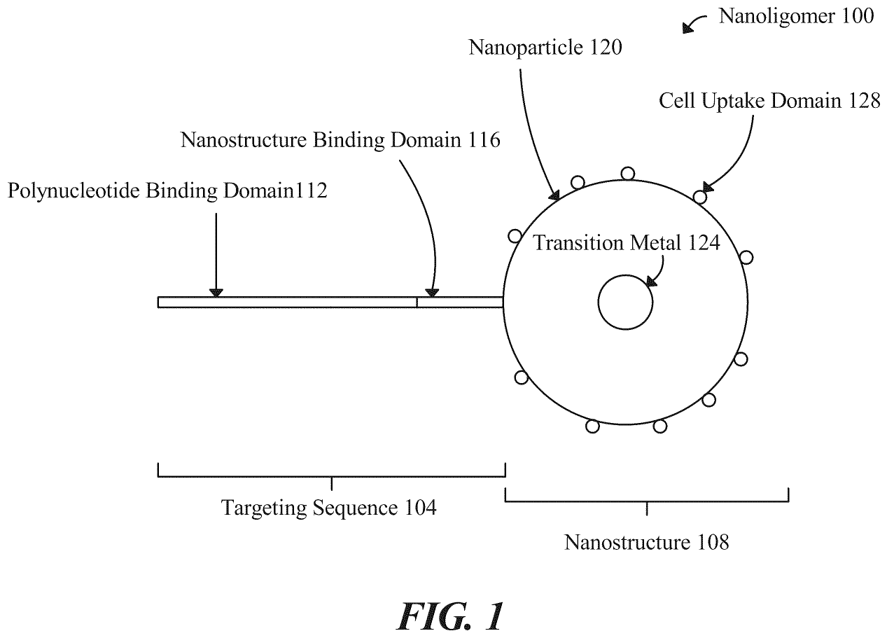

Referring now to FIG. 1, an exemplary embodiment of a system for targeting neuroinflammation is illustrated. A system may include a computing device. A system may include a processor. A processor may include, without limitation, any processor described in this disclosure. A processor may be included in a computing device. A computing device may any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device may include but is not limited to, for example, a computing device or cluster of computing devices in a first location and a second computing device or cluster of computing devices in a second location. Computing device may include one or more computing devices dedicated to data storage, security, distribution of traffic for load balancing, and the like. Computing device may distribute one or more computing tasks as described below across a plurality of computing devices of computing device, which may operate in parallel, in series, redundantly, or in any other manner used for distribution of tasks or memory between computing devices. Computing device may be implemented, as a non-limiting example, using a “shared nothing” architecture.

Still referring to FIG. 1, computing device may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, a nanoligomer 100 may include a targeting sequence 104 and a nanostructure 108. A targeting sequence 104 may include a polynucleotide binding domain 112 and a nanostructure binding domain 116. In some embodiments, a targeting sequence may further include a nuclear localization sequence. In some embodiments, a targeting sequence may further include a transcription activation domain. A nanostructure 108 may include a nanoparticle 120. A nanoparticle 120 may include a transition metal 124. A nanostructure 108 may include a cell uptake domain 128.

Still referring to FIG. 1, in some embodiments, a nanoligomer may include a nanoligomer disclosed in Table 1.

TABLE 1

| Description | Sequence |
|---|---|
| SB_NI_112 mouse | AGTGGTACCGTCTGCTA-AEEA-HHHHH-Au22, Glutathione 18 |
| NFKB1 | |
| SB_NI_112 mouse | CTTCTACTGCTCACAGG-AEEA-HHHHH-Au22, Glutathione 18 |
| NLRP3 | |
| SB_NI_112 human | CGGGTGCTTGCCATCTT-AEEA-HHHHH-Au22, Glutathione 18 |
| NLRP3 | |
| SB_NI_112 human | TGCCATTCTGAAGCCGG-AEEA-HHHHH-Au22, Glutathione 18 |
| NF-κβ | |

Still referring to FIG. 1, in some embodiments, a nanoligomer may include a

nanoligomer disclosed in Table 2.

TABLE 2

| Description | Sequence |
|---|---|
| SB_BGC_CK1-A | A, T, C, A, C, C, A, A, G, T, A, G, |
| | PO, K, F, F, K, F, F, K, F, F, K |
| SB_BGC_CK1-B | T, G, T, G, T, T, A, C, G, C, T, A, |
| | PO, K, F, F, K, F, F, K, F, F, K |
| SB_BGC_CK1-C | G, T, G, A, C, A, T, A, C, A, T, T, |
| | PO, K, F, F, K, F, F, K, F, F, K |

As used in Table 2, A, T, G, and C are nucleotides on a peptide nucleic acid backbone. As used in Table 2, K is Lysine and F is Phenylalanine. As used in Table 2, PO is an AEEA linker. In some embodiments, a composition may include one or more nanoligomers described in Table 2. For example, a composition may include a all 3 nanoligomers described in Table 2.

Still referring to FIG. 1, in some embodiments, a nanoligomer 100 may include a targeting sequence 104. As used herein, a “targeting sequence” is a sequence of nucleobases including a polynucleotide binding domain and a nanostructure binding domain. A targeting sequence may include a peptide nucleic acid (PNA). As used in this disclosure, a “peptide nucleic acid” is a DNA analog comprising a (2-aminoethyl) glycine carbonyl unit, as opposed to a phosphate backbone, that is linked to a nucleotide base by the glycine amino nitrogen and/or methylene linker. In an embodiment, and without limitation, a PNA may include a backbone composed of peptide bonds linking nucleobases. In another embodiment, and without limitation, a PNA may include an amino-terminal and/or a carboxy-terminal end. In another embodiment, and without limitation, a PNA may include a 5′ and/or a 3′ end in the conventional sense, with reference to a complementary nucleic acid sequence to which it specifically hybridizes. In an embodiment, a PNA may include a sequence that may be described in a conventional fashion similar to DNA and/or RNA, such as but not limited to having nucleotides including guanine (G), uracil (U), thymine (T), adenine (A), and/or cytosine (C) which may correspond to a nucleotide sequence of a DNA molecule. In an embodiment, a PNA may be resistant to proteases and/or nucleases as a function of a structural difference from DNA, wherein the structure difference may result in a PNA not being recognized by a hepatic transporter(s) recognizing DNA. In another embodiment, a PNA may comprise at least one modified phosphate backbone such as, but not limited to phosphorothioate, phosphorodithioate, 5-phosphoramidothioate, phosphoramidate, phosphordiamidate, methylphosphonate, alkyl phosphotriester, formacetal, and/or the like thereof. In some embodiments, one or more of a polynucleotide binding domain, a nanoparticle binding domain, a nuclear localization sequence, and a transcription activation domain includes a PNA. A targeting sequence may include a polynucleotide, such as DNA or RNA. A targeting sequence may include an antisense oligonucleotide. As used in this disclosure, an “antisense oligonucleotide” is an antisense molecule that modulates the expression of one or more genes and/or polynucleotides. For example, and without limitation, an antisense oligonucleotide may include antisense PNAs, antisense RNAs, and the like. In another embodiment, antisense oligonucleotides may include RNA and/or DNA oligomers such as but not limited to interfering RNA molecules, such as dsRNA, dsDNA, mRNA, siRNA, and/or hpRNA as well as locked nucleic acids, BNA, polypeptides and/or other oligomers and the like. A nanoligomer may include an inhibitory sequence. An “inhibitory sequence,” as used in this disclosure, is a sequence of nucleotides or other repeating units that acts to suppress a sequence of interest such as a sequence involved in the production of an undesirable protein. In a non-limiting example, an inhibitory sequence may be used to decrease production of neuroinflammatory such as proteins. An inhibitory sequence may include, without limitation, sequences of ribonucleic acid (RNA), deoxyribonucleic acid (DNA), or peptide nucleic acid (PNA). In an embodiment, a targeting sequence is complementary to a sequence of interest. In an mbodiment, an antisense oligonucleotide is complementary to a sequence of interest. In an embodiment, an inhibitory sequence is complementary to a sequence of interest. As used in this disclosure, a “complementary” sequence is a sequence of consecutive nucleobases or semi-consecutive nucleobases capable of hybridizing to another nucleic acid strand or duplex even if less than all the nucleobases base pair with a counterpart nucleobase. In some embodiments, between 70% and 100%, or any range derivable therein, of a nucleobase sequence may be capable of base-pairing with a nucleic acid molecule during hybridization.

Still referring to FIG. 1, a targeting sequence 104 may include a polynucleotide binding domain 112. As used herein, a “polynucleotide binding domain” is a sequence of nucleobases capable of hybridizing with a target polynucleotide. In some embodiments, a polynucleotide binding domain is complementary to a target polynucleotide. In some embodiments, a target polynucleotide encodes a proinflammatory cytokine, a direct inflammasome target, or a transcription factor. In some embodiments, a target polynucleotide encodes TERT, a cytokine selected from the list Interleukin-1β or IL-1β, IL-1α, tumor necrosis factor-alpha or TNF-α, TNF receptor 1 or TNFR1, Interleukin 6 or IL-6, IL-4, and IL-13. In some embodiments, a target polynucleotide encodes an inflammasome target selected from the list NLRP1, NLRP3, NLRC4, AIM2. In some embodiments, a target polynucleotide may encode NLRP3. In some embodiments, a target polynucleotide may encode NF-κβ. In some embodiments, a polynucleotide binding domain targeting mouse NFKB1 has the sequence AGTGGTACCGTCTGCTA. In some embodiments, a polynucleotide binding domain targeting mouse NLRP3 has the sequence CTTCTACTGCTCACAGG. In some embodiments, a polynucleotide binding domain targeting human NLRP3 has the sequence CGGGTGCTTGCCATCTT. In some embodiments, a polynucleotide binding domain targeting human NF-κβ has the sequence TGCCATTCTGAAGCCGG. In some embodiments, a target polynucleotide may include a sequence disclosed in Table 3. In some embodiments, a polynucleotide binding domain may be capable of hybridizing with a sequence disclosed in Table 3.

TABLE 3

| Description | Sequence |
|---|---|
| Human NLRP3 RNA | CGGGTGCTTGCCATCTT |
| Human NLRP3 RNA | GTGCTTGCCATCTTCAT |
| Human NLRP3 RNA | CTTGCCATCTTCATCTG |
| Human NLRP3 RNA | CCATCTTCATCTGCAGC |
| Human NLRP3 RNA | CAGCGGGTGCTTGCCAT |
| Human NF-κβ RNA | TTCTGCCATTCTGAAGC |
| Human NF-κβ RNA | TGCCATTCTGAAGCCGG |
| Human NF-κβ RNA | CCATTCTGAAGCCGGGT |
| Human NF-κβ RNA | ATCATCTTCTGCCATTC |
| Human NF-κβ RNA | ATCTTCTGCCATTCTGA |
| Mouse NFKB1 RNA | AGTGGTACCGTCTGCTA |
| Mouse NLRP3 RNA | CTTCTACTGCTCACAGG |
| SB_BGC_CK1-A | ATCACCAAGTAG |
| SB_BGC_CK1-B | TGTGTTACGCTA |
| SB_BGC_CK1-C | GTGACATACATT |

Still referring to FIG. 1, in some embodiments, a polynucleotide binding sequence may be capable of hybridizing with a section of a sequence in Table 3. In non-limiting examples, a polynucleotide binding sequence may be capable of hybridizing to a 10, 11, 12, 13, 14, 15, 16, or 17 nucleotide long stretch of a sequence in Table 3.

Still referring to FIG. 1, in some embodiments, a polynucleotide binding sequence may include a sequence selected from SEQ ID NO: 1-4. In some embodiments, a polynucleotide binding sequence may be capable of hybridizing with a sequence selected from SEQ ID NO: 5-16.

Still referring to FIG. 1, in some embodiments, a polynucleotide binding sequence may be capable of hybridizing to a viral target sequence. In some embodiments, a viral target sequence may include a target sequence from a DNA virus, such as adenoviruses, herpesviruses, poxviruses, parvoviruses and the like. In some embodiments, a viral target sequence may include a target sequence from an RNA virus, such as influenza, SARS, MERS, COVID-19, Dengue Virus, hepatitis C, hepatitis E, West Nile fever, Ebola virus disease, rabies, polio, mumps, measles, and the like. In some embodiments, a polynucleotide binding sequence capable of hybridizing to a viral target sequence may include a sequence disclosed in Table 4.

TABLE 4

| Description | Sequence |
|---|---|
| α-TRS | TAAAGTTCGTTTAGA |
| α-AUG | GCTCTCCATCTTACC |
| α-FS | ACACCGCAAACCCGT |
| α-PK1 | CGGGCTGCACTTACA |
| α-PK2 | TACTAGTGCCTGTGC |
| α-PK3 | GTATACGACATCAGT |

Still referring to FIG. 1, in some embodiments, a polynucleotide binding domain may target a bacterial polynucleotide. In some embodiments, a polynucleotide binding domain may target a biosynthetic gene cluster polynucleotide. In some embodiments, a polynucleotide binding domain may target a porogymonas gingivali, eubacterium rectale, pseudobutyrivibrio xylanivorans and/or akkermansia muciniphilia polynucleotide.

Still referring to FIG. 1, in some embodiments, a target polynucleotide may include RNA, such as mRNA. In some embodiments, hybridization of a polynucleotide binding sequence to a target mRNA may sterically hinder a ribosomal binding site and downregulate translation from the mRNA. In some embodiments, hybridization of a polynucleotide binding sequence to a target mRNA may prevent translation of the RNA. In some embodiments, a target polynucleotide may include DNA. In some embodiments, hybridization of a polynucleotide binding sequence to a target DNA may prevent transcription of the DNA.

Still referring to FIG. 1, a targeting sequence 104 may include a nanostructure binding domain 116. As used herein, a “nanostructure binding domain” is a nucleobase sequence containing a binding domain for a nanostructure. In some embodiments, a nanostructure binding domain may include a sequence of 5, 6, or 7 consecutive histidine (H in Table 1), 5, 6, or 7 consecutive cysteine, 5, 6, or 7 methionine, 5, 6, or 7 lysine, 5, 6, or 7 glutamine, 5, 6, or 7 arginine, or 5, 6, or 7 asparagine. In some embodiments, a nanostructure binding domain may include a sequence of 5 consecutive histidine. In some embodiments, a nanostructure binding domain may include SEQ ID NO: 18.

Still referring to FIG. 1, a targeting sequence 104 may include a linker. As used herein, a protein “linker” is an amino acid sequence that connects two other sequences. In some embodiments, a linker may include the sequence AEEA. In some embodiments, a linker may include SEQ ID NO: 17. In some embodiments, a spacer may be positioned between nanostructure binding domain and the rest of the targeting sequence.

Still referring to FIG. 1, a nanoligomer may include a nuclear localization sequence. In some embodiments, targeting sequence 104 may include a nuclear localization sequence. As used herein, a “nuclear localization sequence” is an amino acid sequence that increases transport to the nucleus. In some embodiments, a targeting sequence includes a polynucleotide binding sequence targeting DNA and a nuclear localization sequence. In some embodiments, a targeting sequence includes a polynucleotide binding sequence targeting RNA and no nuclear localization sequence. Non-limiting examples of nuclear localization sequences include SV40 sequences such as PKKKRKV, nucleoplasmin sequences such as AVKRPAATKKAGQAKKKKLD, c-Myc sequences such as PAAKRVKLD, EGL-13 sequences such as MSRRRKANPTKLSENAKKLAKEVEN, and TUS-protein sequences such as KLKIKRPVK.

Still referring to FIG. 1, in some embodiments, a targeting sequence 104 may include a transcription activation domain. As used herein, a “transcription activation domain” is an amino acid sequence that stimulates transcription. In some embodiments, inclusion of a transcription activation domain may cause nanoligomer to upregulate transcription of a sequence of interest. A transcription activation sequence may include, without limitation, sequences of ribonucleic acid (RNA), deoxyribonucleic acid (DNA), or peptide nucleic acid (PNA).

Still referring to FIG. 1, in some embodiments, a transcription activation domain may include an acidic domain. As used herein, an “acidic domain” is a sequence at least 3 amino acids in length and including at least 40% acidic amino acids. In some embodiments, a transcription activation domain may be at least 2, 3, 4, 5, 6, 7, 8, 9, 10, or more amino acids in length. In some embodiments, a transcription activation domain may be at most 3, 4, 5, 6, 7, 8, 9, or 10 amino acids in length. In some embodiments, a transcription activation domain may include 10%, 20%, 30%, 40%, 50%, 60%, 70%, 80%, 90%, or 100% acidic amino acids. In some embodiments, a transcription activation domain may include 0.01%-99.9% acidic amino acids. In some embodiments, a transcription activation domain may include a sequence of 3-9 amino acids and at least 40% acidic amino acids. In some embodiments, a transcription activation domain may include a sequence of 3-9 amino acids and at least 50% acidic amino acids.

Acidic domains may be referred to as acid blobs, negative noodles, or nine-amino-acid transactivation domains (9aaTAD). Acidic domains may include a domain common to large superfamilies in eukaryotic transcription factors represented by Gal4, Oaf1, Leu3, Rtg3, Pho4, Gln3, Gcn4 in yeast, and p53, NFAT, NF-κB, and VP16 in mammals. Acidic domains may be rich in D and E amino acids. Acidic domains may have an associated 3 amino acid hydrophobic region next to its N-terminal end. In some embodiments, an acidic domain may include a 9aaTAD disclosed in Table 5.

TABLE 5

| 9 amino acid | | |
|---|---|---|
| sequence | | |
| (9aaTAD) | Source | Peptide-KIX interaction |
| E TFSD LWKL | p53 TAD1 | LSPEETFSDLWKLPE |
| D DIEQ WFTE | p53 TAD2 | QAMDDLMLSPDDIEQWFTEDPGPD |
| S DIMD FVLK | MLL | DCGNILPSDIMDFVLKNTP |
| D LLDF SMMF | E2A | PVGTDKELSDLLDFSMMFPLPVT |
| E TLDF SLVT | Rtg3 | E2A homolog |
| R KILN DLSS | CREB | RREILSRRPSYRKILNDLSSDAP |
| E AILA ELKK | CREBaB6 | CREB-mutant binding to KIX |
| D DVVQ YLNS | Gli3 | TAD homology to CREB/KIX |
| D DVYN YLFD | Gal4 | Pdr1 and Oaf1 homolog |
| D LFDY DFLV | Oaf1 | DLFDYDFLV |
| D FFDY DLLF | Pip2 | Oafl homolog |
| E DLYS ILWS | Pdr1 | EDLYSILWSDWY |
| T DLYH TLWN | Pdr3 | Pdr1 homolog |

Still referring to FIG. 1, in some embodiments, a transcription activation domain may include a glutamine rich domain. As used herein, a “glutamine rich domain” is a sequence at least 3 amino acids in length and including at least 60% glutamine. In some embodiments, a transcription activation domain may be at least 2, 3, 4, 5, 6, 7, 8, 9, 10, or more amino acids in length. In some embodiments, a transcription activation domain may be at most 3, 4, 5, 6, 7, 8, 9, or 10 amino acids in length. In some embodiments, a transcription activation domain may include 10%, 20%, 30%, 40%, 50%, 60%, 70%, 80%, 90%, or 100% glutamine. In some embodiments, a transcription activation domain may include 0.01%-99.9% glutamine. In some embodiments, a transcription activation domain may include a sequence of 3-9 amino acids and at least 60% glutamine. A glutamine rich domain may contain multiple repetitions such as QQQXXXQQQ. As non-limiting examples, glutamine rich domains may include POU2F1 (Oct1), POU2F2 (Oct2), and SP1 (Sp/KLF family). In some embodiments, a glutamine rich domain may include the sequence AQQAQQQQQNQAQQAQQQQQNQ.

Still referring to FIG. 1, in some embodiments, a transcription activation domain may include a proline rich domain. As used herein, a “proline rich domain” is a sequence at least 3 amino acids in length and including at least 60% proline. In some embodiments, a transcription activation domain may be at least 2, 3, 4, 5, 6, 7, 8, 9, 10, or more amino acids in length. In some embodiments, a transcription activation domain may be at most 3, 4, 5, 6, 7, 8, 9, or 10 amino acids in length. In some embodiments, a transcription activation domain may include 10%, 20%, 30%, 40%, 50%, 60%, 70%, 80%, 90%, or 100% proline. In some embodiments, a transcription activation domain may include 0.01%-99.9% proline. In some embodiments, a transcription activation domain may include a sequence of 3-9 amino acids and at least 60% proline. A proline rich domain may contain multiple repetitions such as PPPXXXPPP. As non-limiting examples, proline rich domains may include c-jun, AP2, and/or Oct-2. In some embodiments, a proline rich domain may include the sequence PPPDLGPPPDLGPPP.

Still referring to FIG. 1, in some embodiments, a transcription activation domain may include an isoleucine rich domain. As used herein, a “isoleucine rich domain” is a sequence at least 3 amino acids in length and including at least 50% isoleucine. In some embodiments, a transcription activation domain may be at least 2, 3, 4, 5, 6, 7, 8, 9, 10, or more amino acids in length. In some embodiments, a transcription activation domain may be at most 3, 4, 5, 6, 7, 8, 9, or 10 amino acids in length. In some embodiments, a transcription activation domain may include 10%, 20%, 30%, 40%, 50%, 60%, 70%, 80%, 90%, or 100% isoleucine. In some embodiments, a transcription activation domain may include 0.01%-99.9% isoleucine. In some embodiments, a transcription activation domain may include a sequence of 3-9 amino acids and at least 50% isoleucine. An isoleucine rich domain may contain multiple repetitions such as IIXXII. As a non-limiting example, an isoleucine rich domain may include NTF-1. In some embodiments, an isoleucine rich domain may include the sequence KSHAHAQKRIRRRLLIILL.

Still referring to FIG. 1, in some embodiments, which transcription activation domain is used may vary with promotor sequence. For example, many acidic domains may act using TATA-binding factors or TAFs (TATA-binding protein-associated factors), while other transcription activation domains may use a different mechanism of action, making them better suited for cases when a TATA-box is not present and GC and/or CCAAT boxes are found. As a non-limiting example, VP16 may act via the acidic region which may interact with TATA binding factor TFIID. Acidic domains such as VP16 may be prioritized when TATA-box is available for a target polynucleotide. As another example, glutamine rich domains may bind to GC rich sequence elements and may be prioritized when GC boxes/promoters are present. In another example, proline rich domains may interact with CCAAT box, and may be prioritized when a CCAAT box is present. In some embodiments, an isoleucine rich domain may be used when TATA-box binding protein associated factors are present.

Still referring to FIG. 1, in some embodiments, a eukaryotic activation domain may include a promoter selected from the list consisting of CMV (Strong mammalian promoter from human cytomegalovirus), EFla (Strong mammalian promoter from human elongation factor 1 alpha), CAG (Strong hybrid mammalian promoter), PGK (Mammalian promoter from phospholycerate kinase gene), TRE (Tetracycline response element promoter), U6 (Human U6 nuclear promoter for small RNA expression), and UAS (Drosophila promoter containing Gal4 binding sites). In some embodiments, a bacterial activation domain may include a promoter selected from the list consisting of T7, Sp6, lac, araBad, trp, and Ptac.

Still referring to FIG. 1, in some embodiments, a nanoligomer 100 may include a nanostructure 108. In some embodiments, a nanoligomer may include a targeting sequence bound to a nanostructure. As used in this disclosure a “nanostructure” is a structure of intermediate size between microscopic and molecular structures. For example, and without limitation, nanostructure may include a structure that comprises a size in the range of 0.1 nm to 100 nanometers. In an embodiment, and without limitation, a nanostructure 108 may include a nanoparticle 120. In an embodiment, and without limitation, a targeting sequence may be bound to a nanostructure via a covalent bond. As used in this disclosure a “covalent bond” is a chemical bond that involves sharing electrons between atoms. For example, and without limitation, covalent bond may include electron pairs that are shared and/or bonded as a function of a stable balance of attractive and/or repulsive forces between atoms. In an embodiment, and without limitation, a covalent bond may allow molecules and/or atoms to fill one or more valence shells of an atom to produce a stable electronic configuration. In another embodiment, a covalent bond may include one or more interactions such as, but not limited to σ-bonding, ϕ-bonding, metal-to-metal bonding, agnostic interactions, bent bonds, three-center two-electron bonds, three-center four-electron bonds, and the like.

Still referring to FIG. 1, a nanostructure 108 may include a nanoparticle 120. As used herein a “nanoparticle” is a three-dimensional object existing on a nanoscale, wherein the particle is between 0.1 nm and 100 nm in each spatial dimension. A nanoparticle may be spherical. For example, and without limitation, a nanoparticle may include a spherical nanoparticle with a diameter of about 23 nm.

Still referring to FIG. 1, in some embodiments, a nanoparticle 120 may include a transition metal. In some embodiments, a nanoparticle may include gold. In some embodiments, a nanoparticle may include Au 22. As used in this disclosure a “transition metal nanoparticle” is a nanoparticle composed of a transition metal. For example, and without limitation, a transition metal nanoparticle may include a gold nanoparticle. As a further non-limiting example, a transition metal nanoparticle may include a copper nanoparticle. As a further non-limiting example, a transition metal nanoparticle may include a zinc nanoparticle. In an embodiment, and without limitation, a transition metal nanoparticle may include one or more transition metals comprising groups 3-12 transition metals on the period table of elements.

Still referring to FIG. 1, in some embodiments, and without limitation, pharmaceutical composition may include a lipid nanoparticle. A lipid nanoparticle (LNP) may comprise a therapeutic oligomer, a cationic lipid, an aggregation reducing agent, such as but not limited to polyethylene glycol (PEG) lipid and/or PEG-modified lipid, and a non-cationic lipid such as but not limited to a neutral lipid, a sterol, and the like. In some embodiments, a lipid nanoparticle may include an outer lipid membrane and an inner aqueous center. In some embodiments, lipid nanoparticle may include any morphology generated as a function of combining a cationic lipid and one or more further lipids in an aqueous environment and/or in the presence of a therapeutic oligomer. For example, and without limitation, lipid nanoparticle may include a liposome, a lipid complex, a lipoplex, an emulsion, a micelle, a lipidic nanocapsule, a nanosuspension and the like. In an embodiment, and without limitation, lipid nanoparticle may include at least one cationic lipid, a neutral lipid, a sterol such as but not limited to cholesterol, and/or a PEG-lipid, wherein lipid nanoparticle may comprise a molar ratio of 20-60% cationic lipid, 5-25% neutral lipid, 25-55% sterol, and/or 0.5-15% PEG-lipid. LNP may be attached to targeting sequence and/or one or more cell uptake domains. In some embodiments, lipid nanoparticle may include at least 20%, 25%, 30%, 35%, 40%, 45%, 50%, or 55% cationic lipid. In some embodiments, lipid nanoparticle may include at least 5%, 10%, 15%, or 20% neutral lipid. In some embodiments, lipid nanoparticle may include at least 25%, 30%, 35%, 40%, 45%, or 50% sterol. In some embodiments, lipid nanoparticle may include at least 0.5%, 0.6%, 0.7%, 0.8%, 0.9%, 1%, 1.1%, 1.2%, 1.3%, or 1.4% PEG-lipid. Additionally, or alternatively, a pharmaceutical composition may include an amino alcohol lipidoid.

Still referring to FIG. 1, lipid nanoparticles may include any cationic lipid suitable for forming a lipid nanoparticle. In an embodiment, and without limitation, cationic lipid may comprise a net positive charge at a physiological pH. In another embodiment, and without limitation, cationic lipid may be an amino lipid. As used in this disclosure an “amino lipid” is a lipid comprising at least a fatty acid and/or fatty alkyl chains and an amino head group such as, but not limited to, an alkylamino or dialkylamino group, wherein the amino head group may be protonated to form a cationic lipid at physiological pH. In an embodiment, and without limitation, cationic lipid may be, for example, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA),1,2-Dilinolenyloxy-N,N-dimethylaminopropane (DLenDMA),1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC),1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP),1,2-Dilinoleyloxy-3-trimethylaminopropane chloride salt (DLin-TMA·Ci),1,2-Dilinoleoyi-3-trimethylaminopropane chloride salt (DLin-TAP·CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP),1,2-Dilinoleyloxo-3-(2-N,N-dimethylamino)ethoxypropane (DLin-EG-DMA), 2,2-Dilinoleyl-4-dimethylaminomethyl-[1,3]-dioxolane(DLin-K-DMA) and/or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl 4-(dimethylamino)butanoate (MC3), 1,1′-(2-(4-(2-((2-(bis(2-hydroxydodecyl)amino)ethyl)(2-hydroxydodecyl)amino)ethyl)piperazin-1 yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-DMA), (6Z,9Z,28Z,31Z)-heptatriaconta-6,9,28,31-tetraen-19-yl 4-(dimethylamino) butanoate (DLin-M-C3-DMA), 3 -((6Z, 9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), and the like thereof. Additionally or alternatively, cationic lipids may include, but are not limited to, N,N-distearyl-N,N-dimethylammonium bromide (DDAB), 3P-(N-(N′,N′dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3 phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), 2,2-Dilinoleyl-4-dimethylaminoethyl-[1,3]-dioxolane (XTC), and the like thereof. In another embodiment cationic lipid may be selected from the group consisting of 98N12-5, C12-200, and/or ckk-E12.

Still referring to FIG. 1, cationic lipid may also be an amino lipid such as but not limited to amino lipids that comprise alternative fatty acid groups and/or other dialkylamino groups, including those in which the alkyl substituents are different (e.g., N-ethyl-N-methylamino-, and N-propyl-N-ethylamino-). In an embodiment, and without limitation amino lipids having less saturated acyl chains may be more easily sized for purposes of filter sterilization. Amino lipids containing unsaturated fatty acids with carbon chain lengths in the range of C14 to C22 may be used. Other scaffolds may also be used to separate the amino group and/or the fatty acid and/or fatty alkyl portion of the amino lipid. For example, and without limitation, amino lipids may include 1,2-dilinoleyoxy-3-(dimethylamino)acetoxypropane (DLin-DAC),1,2-dilinoleyoxy-3-morpholinopropane (DLin-MA),1,2-dilinoleoyl-3-dimethylaminopropane (DLinDAP), 1,2-dilinoleylthio-3-dimethylaminopropane (DLin-S-DA),1-linoleoyl-2-linoleyloxy-3 dimethylaminopropane (DLin-2-DMAP),1,2-dilinoleyloxy-3-trimethylaminopropane chloride salt (DLin-TMA·CI),1,2-dilinoleoyl-3-trimethylaminopropane chloride salt (DLin-TAP·CI), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP),1,2-dilinoleyloxo-3-(2-N,N-dimethylamino)ethoxypropane (DLin-EG-DMA), 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA), dilinoleyl-methyl-4-dimethylaminobutyrate (DLin-MC3-DMA), and the like.

Still referring to FIG. 1, in some embodiments, amino and/or cationic lipids may have at least one protonatable and/or deprotonatable group, such that the lipid is positively charged at a pH at or below physiological pH (e.g. pH 7.4) and neutral at a second pH, wherein second pH may be at or above physiological pH. In an embodiment, addition and/or removal of protons as a function of pH is an equilibrium process, wherein the reference to a charged and/or a neutral lipid refers to the nature of the predominant species and does not require that all of the lipid be present in the charged or neutral form. In an embodiment, and without limitation, lipids that have more than one protonatable or deprotonatable group, and/or are zwitterionic, may be selected. In another embodiment, and without limitation, protonatable lipids may have a pKa of the protonatable group in the range of 4 to 11, such as but not limited to a pKa of 5 to 7. Lipid nanoparticles may include two or more cationic lipids. The cationic lipids may be selected to contribute different advantageous properties. For example, and without limitation, cationic lipids that differ in properties such as amine pKa, chemical stability, half-life in circulation, half-life in tissue, net accumulation in tissue, and/or toxicity may be used in the formulation of lipid nanoparticle. As a further non-limiting example, cationic lipids may be chosen so that the properties of the mixed-lipid nanoparticle may be more desirable than the properties of a single-lipid nanoparticle of individual lipids.

Still referring to FIG. 1, cationic lipid may be present in a ratio of 20 mol % to 70 or 75 mol %, or from 45 to 65 mol %, or 20, 25, 30, 35, 40, 45, 50, 55, 60, 65, or 70 mol % of the total lipid present in the lipid nanoparticle. In another embodiment, and without limitation lipid nanoparticles may comprise from 25% to 75% on a molar basis of cationic lipid, e.g., from 20 to 70%, from 35 to 65%, from 45 to 65%, 60%, 57.5%, 57.1%, 50% or 40% on a molar basis (based upon 100% total moles of lipid in the lipid nanoparticle). In another embodiment, and without limitation, the ratio of cationic lipid to nucleic acid may be from 3 to 15, such as from 5 to 13 or from 7 to 11. The amount of the permanently cationic lipid or lipidoid may be selected as a function of the nucleic acid concentration. In an embodiment, and without limitation, these amounts may be selected to result in an N/P ratio of the nanoparticle(s) or of the composition in the range from 0.1 to 20. As used in this disclosure an “N/P ratio” is the mole ratio of the nitrogen atoms (“N”) of the basic nitrogen-containing groups of the lipid and/or lipidoid to the phosphate groups (“P”) of therapeutic oligomer. The N/P ratio may be calculated on the basis that, for example, Ipg RNA may contain 3 nmol phosphate residues, provided that targeting sequence exhibits a statistical distribution of bases. The “N”-value of the lipid or lipidoid may be calculated on the basis of its molecular weight and the relative content of permanently cationic and/or cationisable groups. In another embodiment, and without limitation, the LNP comprises one or more additional lipids which stabilize the formation of particles during their formation.

Still referring to FIG. 1, pharmaceutical composition may include a non-cationic lipid, wherein non-cationic lipid may be a neutral lipid, an anionic lipid, an amphipathic lipid, and the like. In an embodiment, and without limitation, neutral lipids may include any of a number of lipid species that exist either in an uncharged and/or neutral zwitterionic form at physiological pH. For example, and without limitation, non-cationic lipids may include diacylphosphatidylcholine, diacylphosphatidylethanolamine, ceramide, sphingomyelin, dihydrosphingomyelin, cephalin, cerebrosides, and the like. In an embodiment, and without limitation, neutral lipids may be selected as a function of stabilizing LNP size and stability in a subject's bloodstream. In another embodiment, and without limitation, the neutral lipid may be a lipid having two acyl groups (e.g., diacylphosphatidylcholine and diacylphosphatidylethanolamine). In an embodiment, and without limitation, neutral lipids may contain saturated fatty acids with carbon chain lengths in the range of C10 to C20. In another embodiment, and without limitation, neutral lipids may include mono and/or diunsaturated fatty acids with carbon chain lengths in the range of C10 to C20. Additionally or alternatively, neutral lipids may comprise mixtures of saturated and/or unsaturated fatty acid chains. For example, and without limitation, neutral lipids may include distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl- phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dimyristoyl phosphatidylcholine (DMPC), distearoyl-phosphatidyl-ethanolamine (DSPE), SM, 16-O-monomethyl PE, 16-O-dimethyl PE, 18-1-trans PE, 1-stearoyl-2-oleoyl-phosphatidyethanolamine (SOPE), cholesterol, and the like thereof. As a further non-limiting example, anionic lipids may include phosphatidylglycerol, cardiolipin, diacylphosphatidylserine, diacylphosphatidic acid, N-dodecanoyl phosphatidylethanoloamine, N-succinylphosphatidylethanolamine, N-glutaryl phosphatidylethanolamine, lysylphosphatidylglycerol, and the like thereof. In an embodiment, and without limitation neutral lipid may be 1,2-distearoyl-sn-glycero-3phosphocholine (DSPC). In another embodiment, and without limitation, LNPs may comprise a neutral lipid selected from DSPC, DPPC, DMPC, DOPC, POPC, DOPE, SM, and the like thereof. In another embodiment, and without limitation, the molar ratio of the cationic lipid to the neutral lipid may range from 2:1 to 8:1. Amphipathic lipids may refer to any suitable material, wherein the hydrophobic portion of the lipid material orients into a hydrophobic phase, while the hydrophilic portion orients toward the aqueous phase. In an embodiment, and without limitation, amphipathic lipids may include phospholipids, aminolipids, and/or sphingolipids. As a further non-limiting example, phospholipids may include sphingomyelin, phosphatidylcholine, phosphatidylethanolamine, phosphatidylserine, phosphatidylinositol, phosphatidic acid, paimitoyloleoyl phosphatdylcholine, phosphatidylcholine, lysophosphatidylethanolamine, dipalmitoylphosphatidylcholine, dioleoylphosphatidylcholine, distearoylphosphatidylcholine, dilinoleoylphosphatidylcholine, and the like. As a further non-limiting example, amphipathic lipids may include any other phosphorus-lacking compounds, such as but not limited to sphingolipids, glycosphingolipid families, diacylglycerols, and beta-acyloxyacids.

Still referring to FIG. 1, non-cationic lipid may be present in a ratio of from 5 mol % to 90 mol %, 5 mol % to 10 mol %, about 5, 10, 15, 20, 25, 30, 35, 40, 45, 50, 55, 60, 65, 70, 75, 80, 85, or about 90 mol % of the total lipid present in the LNP. In an embodiment, and without limitation, LNPs may comprise from 0% to 15 or 45% on a molar basis of neutral lipid, e.g., from 3 to 12% or from 5 to 10%. For example, and without limitation, LNPs may include about 15%, about 10%, about 7.5%, or about 7.1% of neutral lipid on a molar basis, wherein the percentage may be based upon 100% total moles of lipid in the LNP. In an embodiment, and without limitation, LNP may be formed as a function of a sterol. For example, and without limitation, sterol may include a cholesterol. In an embodiment, and without limitation, sterol may be present in a ratio of 10 mol % to 60 mol % or 25 mol % to 40 mol % of the LNP. In an embodiment, and without limitation, the sterol may be present in a ratio of about 10, 15, 20, 25, 30, 35, 40, 45, 50, 55, or about 60 mol % of the total lipid present in the LNP. In another embodiment, and without limitation, LNPs may comprise from 5% to 50% on a molar basis of the sterol, e.g., 15% to 45%, 20% to 40%, about 48%, about 40%, about 38.5%, about 35%, about 34.4%, about 31.5% or about 31% on a molar basis, wherein the percentage may be based upon 100% total moles of lipid in the LNP.

Still referring to FIG. 1, LNP may be formed as a function of an aggregation reducing agent. The aggregation reducing agent may be a lipid capable of reducing aggregation. For example, and without limitation, aggregation reducing agent may include polyethylene glycol (PEG)-modified lipids, monosialoganglioside Gml, polyamide oligomers (PAO), and the like thereof. As a further non-limiting example, aggregation reducing agent may include one or more compounds with uncharged, hydrophilic, steric-barrier moieties, which may prevent aggregation during formulation as a function of being coupled to a lipid, such as but not limited to PEG, Gml, and/or ATTA. In an embodiment, and without limitation, aggregation reducing agent may be, for example, selected from a polyethyleneglycol (PEG)-lipid including, without limitation, a PEG-diacylglycerol (DAG), a PEG-dialkylglycerol, a PEG-dialkyloxypropyl (DAA), a PEG-phospholipid, a PEG-ceramide (Cer), or a mixture thereof, such as but not limited to PEG-Cer14 and/or PEG-Cer20. The PEG-DAA conjugate may be, for example, a PEG-dilauryloxypropyl (C12), a PEG-dimyristyloxypropyl (C14), a PEG-dipalmityloxypropyl (C16), or a PEG-distearyloxypropyl (C18). Other pegylated-lipids may include, but are not limited to, polyethylene glycol-didimyristoyl glycerol (C14-PEG or PEG-C14, where PEG has an average molecular weight of 2000 Da) (PEG-DMG); (R)-2,3 bis(octadecyloxy)propyl-1-(methoxy polyethylene glycol)2000)propylcarbamate)(PEG-DSG); PEG-carbamoyl-1,2-dimyristyloxypropylamine, in which PEG has an average molecular weight of 2000 Da (PEG) cDMA); N-Acetylgalactosamine-((R)-2,3-bis(octadecyloxy)propyl-1-(methoxy polyethylene glycol)2000)propylcarbamate))(GalNAc-PEG-DSG); mPEG (mw2000)-diastearoylphosphatidyl-ethanolamine (PEG-DSPE); and polyethylene glycol-dipalmitoylglycerol (PEG-DPG). In an embodiment, and without limitation, the aggregation reducing agent may be PEG-DMG. In other embodiments, the aggregation reducing agent may be PEG-c-DMA.

Still referring to FIG. 1, the molar ratio of the cationic lipid to the PEGylated lipid may range from 100:1 to 25:1. In an embodiment and without limitation, the composition of LNPs may be influenced by, inter alia, the selection of the cationic lipid component, the degree of cationic lipid saturation, the nature of the PEGylation, the ratio of all components, and/or biophysical parameters such as but not limited to size. In another embodiment, and without limitation, LNPs may comprise from 35 to 45% cationic lipid, from 40% to 50% cationic lipid, from 50% to 60% cationic lipid and/or from 55% to 65% cationic lipid. In an embodiment, and without limitation, the ratio of lipid to therapeutic oligomer 112 may range from 5:1 to 20:1, from 10:1 to 25:1, from 15:1 to 30:1 and/or at least 30:1. The average molecular weight of the PEG moiety in the PEG-modified lipids can range from 500 to 8,000 Daltons, such as but not limited to 1,000 to 4,000 Daltons. In another embodiment, and without limitation, the average molecular weight of the PEG moiety may be about 2,000 Daltons. In an embodiment, and without limitation the concentration of aggregation reducing agent may range from 0.1 to 15 mol %, per 100% total moles of lipid in the LNP. In an embodiment, and without limitation, LNPs include less than 3, 2, or 1 mole percent of PEG or PEG-modified lipid, based on the total moles of lipid in the LNP. In further embodiments, LNPs may comprise from 0.1% to 20% of the PEG-modified lipid on a molar basis, e.g., about 0.5 to about 10%, about 0.5 to about 5%, about 10%, about 5%, about 3.5%, about 3%, about 2,5%, about 2%, about 1.5%, about 1%, about 0.5%, or about 0.3% on a molar basis, wherein the percentage is based on 100% total moles of lipids in the LNP. As a non-limiting example. different LNPs may have varied molar ratios of cationic lipid, non-cationic (or neutral) lipid, sterol (e.g., cholesterol), and/or aggregation reducing agent, such as but not limited to a PEG-ylated lipid, on a molar basis based upon the total moles of lipid in the lipid nanoparticles. Additionally or alternatively, the total amount of nucleic acid, particularly the one or more therapeutic oligomers in the lipid nanoparticles may vary and/or may be defined depending on the therapeutic oligomer to total lipid w/w ratio. For example, and without limitation, targeting sequence to total lipid ratio may be less than 0.06 w/w and/or between 0.03 w/w and 0.04 w/w.

Still referring to FIG. 1, LNPs may occur as liposomes and/or lipoplexes as described in further detail below. In an embodiment, and without limitation, LNPs may have a median diameter size of from 50 nm to 300 nm, such as from 50 nm to 250 nm, for example, from 50 nm to 200 nm. In an embodiment, and without limitation, smaller LNPs may be used. For example, and without limitation, particles may comprise a diameter from below 0.1 pm up to 100 nm such as, but not limited to, less than 0.1 pm, less than 1.0 pm, less than 5 pm, less than 10 pm, less than 15 pm, less than 20 pm, less than 25 pm, less than 30 pm, less than 35 pm, less than 40 pm, less than 50 pm, less than 55 pm, less than 60 pm, less than 65 pm, less than 70 pm, less than 75 pm, less than 80 pm, less than 85 pm, less than 90 pm, less than 95 pm, less than 100 pm, less than 125 pm, less than 150 pm, less than 175 pm, less than 200 pm, less than 225 pm, less than 250 pm, less than 275 pm, less than 300 pm, less than 325 pm, less than 350 pm, less than 375 pm, less than 400 pm, less than 425 pm, less than 450 pm, less than 475 pm, less than 500 pm, less than 525 pm, less than 550 pm, less than 575 pm, less than 600 pm, less than 625 pm, less than 650 pm, less than 675 pm, less than 700 pm, less than 725 pm, less than 750 pm, less than 775 pm, less than 800 pm, less than 825 pm, less than 850 pm, less than 875 pm, less than 900 pm, less than 925 pm, less than 950 pm, less than 975 pm, and the like thereof. In another embodiment, nucleic acids may be delivered using smaller LNPs which may comprise a diameter from 1 nm to 100 nm, from lnm to l0 nm, lnm to 20 nm, from 1 nm to 30 nm, from lnm to 40 nm, from lnm to 50 nm, from 1 nm to 60 nm, from lnm to 70 nm, from lnm to 80 nm, from 1 nm to 90 nm, from 5 nm to from 100 nm, from 5 nm to 10 nm, 5 nm to 20 nm, from 5 nm to 30 nm, from 5 nm to 40 nm, from 5 nm to 50 nm, from 5 nm to 60 nm, from 5 nm to 70 nm, from 5 nm to 80 nm, from 5 nm to 90 nm, 10 to 50 nm, from 20 to 50 nm, from 30 to 50 nm, from 40 to 50 nm, from 20 to 60 nm, from 30 to 60 nm, from 40 to 60 nm, from 20 to 70 nm, from 30 to 70 nm, from 40 to 70 nm, from 50 to 70 nm, from 60 to 70 nm, from 20 to 80 nm, from 30 to 80 nm, from 40 to 80 nm, from 50 to 80 nm, from 60 to 80 nm, from 20 to 90 nm, from 30 to 90 nm, from 40 to 90 nm, from 50 to 90 nm, from 60 to 90 nm and/or from 70 to 90 nm. In an embodiment, and without limitation, the LNP may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

Still referring to FIG. 1, LNPs may have a single mode particle size distribution, wherein a single mode may denote that the particle size distribute is not bi-modal and/or poly-modal. In an embodiment, and without limitation, one or more alternative lipids may be included in the liposome compositions for a variety of purposes, such as to prevent lipid oxidation and/or to attach ligands onto the liposome surface. Any of a number of lipids may be present in LNPs, including amphipathic, neutral, cationic, and anionic lipids. Such lipids may be used alone or in combination. Additional components that may be present in an LNP include bilayer stabilizing components such as polyamide oligomers, peptides, proteins, detergents, and the like.

Still referring to FIG. 1, pharmaceutical compositions may be formulated as liposomes. In an embodiment, and without limitation, cationic lipid-based liposomes may be able to complex with negatively charged nucleic acids, such as but not limited to therapeutic oligomers, via electrostatic interactions, which may result in complexes that offer biocompatibility, low toxicity, and/or the possibility of the large-scale production required for in vivo clinical applications. Liposomes may fuse with the plasma membrane for uptake, wherein once inside the cell, the liposomes may be processed via the endocytic pathway, and wherein the nucleic acid may then be released from the endosome/carrier into the cytoplasm. In an embodiment, and without limitation, liposomes may increase one or more biocompatibilities as a function of a biological membrane similarity, wherein liposomes may be prepared from both natural and/or synthetic phospholipids. In an embodiment, and without limitation, liposomes may consist of a lipid bilayer that may be composed of cationic, anionic, or neutral (phospho)lipids and/or cholesterol, which may enclose an aqueous core. Both the lipid bilayer and the aqueous space may incorporate hydrophobic and/or hydrophilic compounds, respectively. Liposomes may have one or more lipid membranes. Liposomes may be single-layered and/or unilamellar and/or multi-layered and/or multilamellar. Liposome characteristics and/or behavior in vivo may be modified by addition of a hydrophilic polymer coating, such as but not limited to a polyethylene glycol (PEG), to the liposome surface which may confer steric stabilization. In another embodiment, liposomes may be used for specific targeting by attaching ligands, such as but not limited to antibodies, peptides, and/or carbohydrates, to the surface and/or to the terminal end of the attached PEG chains. In another embodiment, liposomes may be spherical vesicles and may range in size from 20 nm to 100 microns. In another embodiment, liposomes may be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

Still referring to FIG. 1, pharmaceutical composition may be formulated in the form of lipoplexes, such as but not limited to cationic lipid bilayers sandwiched between nucleic acid and/or therapeutic oligomer layers. Cationic lipids, such as DOTAP, (1,2) dioleoyl-3-trimethylammonium-propane) and DOTMA (N-[1-(2,3-dioleoyloxy)propyl]-N,N,N-trimethyl-ammonium methyl sulfate) may form complexes and/or lipoplexes with negatively charged nucleic acids to form nanoparticles by electrostatic interaction, which may provide high in vitro transfection efficiency. Additionally or alternatively, pharmaceutical composition may be formulated in the form of nanoliposomes, such as but not limited to neutral lipid-based nanoliposomes such as 1,2-dioleoyl-sn-glycero-3-phosphatidylcholine (DOPC)-based nanoliposomes. Additionally or alternatively, pharmaceutical composition may be formulated in the form of an emulsion. In an embodiment, and without limitation, emulsion may include a cationic oil-in-water emulsion, wherein the emulsion particle comprises an oil core and a cationic lipid which may interact with targeting sequence, wherein the therapeutic oligomer may be anchored to the emulsion particle. In an embodiment, and without limitation, emulsion may include a water-in-oil emulsion comprising a continuous hydrophobic phase in which the hydrophilic phase may be dispersed.

Still referring to FIG. 1, in some embodiments, a nanostructure 108 may include a cell uptake domain 128. As used herein, a “cell uptake domain” is a sequence of 1 to 3 amino acids that increases uptake by a cell type of interest. In some embodiments, a cell uptake domain may modulate the degree to which a nanoligomer is taken up by a tissue or organ of interest. For example, certain cell uptake domains may increase nanoligomer uptake in the brain. In some embodiments, a cell uptake domain may be attached to the outside of a nanostructure. In some embodiments, a cell uptake domain may use a stable thiol-amine covalent bond to attach to a nanostructure surface. In some embodiments, this bond type may avoid cleavage or dissociation as nanoligomers transport through different tissues and cell regions. In some embodiments, a nanoligomer may include multiple cell uptake domains.

Still referring to FIG. 1, in some embodiments, a cell uptake domain may include a sequence of 1-3 amino acids. In some embodiments, a cell uptake domain may include a sequence of 3 amino acids, such as 3 of the same amino acid. Without wishing to be bound by theory, limiting a cell uptake domain to 3 or fewer amino acids may avoid creating an immune response.

Still referring to FIG. 1, in some embodiments, a cell uptake domain may cause selective uptake and/or active diffusion of a nanoligomer. In some embodiments, inclusion of certain amino acids in a cell uptake domain may cause uptake by varying tissues and/or organs. Examples of cell uptake domain amino acids and their associated organs are disclosed in Table 6.

TABLE 6

| Target location | Cell uptake domain |
|---|---|
| Brain | Glycine, glutamic acid, glutamine, histidine, tyrosine, |
| | GABA, tryptophan, arginine |
| Liver | Cysteine, glycine, methionine, glutamine, arginine |
| Kidney | Arginine, carnitine |
| Heart | Arginine, carnitine, taurine, citrulline |
| Lungs | Glutathione (reduced), cysteine, glutamic acid, glycine |
| Stomach | Glycine, glutamic acid, histidine |
| GI tract | Glycine, glutamine, alanine, serine |
| Circulation | Arginine, carnitine, taurine |
| Muscle | Glycine, glutamine, valine, leucine, isoleucine, alanine |
| Connective tissue | Glycine, lysine, proline, carnosine |

Still referring to FIG. 1, as a non-limiting example, a tri-peptide for uptake into organs including the brain, liver, lung, stomach, GI tract, muscle, connective tissue may include cysteine, glutamic acid, and glycine. In some embodiments, a cell uptake domain including cysteine, glutamic acid, and glycine or reduced glutathione may be used for an NLRP3 and/or NFKB specific nanoligomer. Replacing glycine with arginine may be used for more specific delivery to kidney, heart, and circulation. In some embodiments, a cell uptake domain may include glutathione. In some embodiments, a cell uptake domain may include glutathione 18.

Still referring to FIG. 1, there are also size considerations relevant to cell uptake that apply both to the cell uptake domain and to the rest of the nanoligomer. In some embodiments, the hydrodynamic size of a nanoligomer may be restricted to about 5 nm. As non-limiting examples, the hydrodynamic size of a nanoligomer may be 5 nm, or less than 5 nm. As used herein, a “hydrodynamic size” of an object is a size of a perfect solid sphere that would exhibit the same hydrodynamic friction as the object. Without wishing to be bound by theory, this may prevent accumulation in first pass-organs such as the liver, kidney and spleen, and may allow for easier clearance of non-bound nanoligomers. In some embodiments, a nanoligomer designed for brain penetration may have a hydrodynamic size of less than 2 nm. In some embodiments, peripherally restricted nanoligomers may be 4 to 5 nm.

Still referring to FIG. 1, there are also charge considerations relevant to cell uptake that apply both to the cell uptake domain and to the rest of the nanoligomer. In some embodiments, positive and negatively charged molecules may form a protein corona in serum, which may cause them to exceed the size ranges listed above. In some embodiments, nanoligomer components may be designed using moieties that are either neutral in charge (such as peptide nucleic acids) or zwitterionic (such as cysteine). As non-limiting examples, a targeting sequence may include peptide nucleic acids and a LNP may include only neutral and zwitterionic lipids or primarily neutral and zwitterionic lipids. In some embodiments, this may avoid drawing oppositely charged components in biological media and tissues or activating an immune response. In some embodiments, the exterior of a nanoligomer is primarily neutral in charge. In some embodiments, a nanoligomer does not form a protein corona in serum.

Still referring to FIG. 1, in some embodiments, a composition, such as a therapeutic composition, may include a nanoligomer. In some embodiments, a composition may include more than one type of nanoligomer. In some embodiments, a composition may include a first nanoligomer including a targeting sequence targeting NLRP3 and a second nanoligomer including a targeting sequence targeting NF-κβ. In some embodiments, a composition including a first nanoligomer and a second nanoligomer may have a 1:1 ratio of nanoligomers. For example, a composition may have a 1:1 ratio of nanoligomers targeting NLRP3 and nanoligomers targeting NF-κβ.

Still referring to FIG. 1, in some embodiments, a nanoligomer 100 may be capable of regulating the expression of a target gene. In some embodiments, a nanoligomer may be capable of upregulating transcription of a target gene. In some embodiments, a nanoligomer may be capable of downregulating transcription of a target gene. In some embodiments, a nanoligomer may be capable of downregulating expression of a gene by regulating mRNA translation. In some embodiments, a nanoligomer may be capable of downregulating expression of a gene by decreasing RNA stability.

Still referring to FIG. 1, in some embodiments, administration of a nanoligomer 100 to a subject may have a therapeutic or prophylactic effect. As used herein, a “therapeutic effect” is a reduction or elimination of a symptom of a disease in a subject. In some embodiments, administration of a nanoligomer to a subject may result in a therapeutic or prophylactic effect in the central nervous system. In some embodiments, administration of a nanoligomer to a subject may result in a therapeutic or prophylactic effect in the brain. In some embodiments, a nanoligomer is capable of crossing the blood brain barrier.

Still referring to FIG. 1, in some embodiments, a nanoligomer 100 may be capable of regulating the NLRP3 inflammasome. In some embodiments, a nanoligomer may be capable of downregulating expression of the NLRP3 gene. In some embodiments, a nanoligomer may include a targeting sequence capable of hybridizing with a polynucleotide encoding NLRP3. In some embodiments, a nanoligomer may include a targeting sequence capable of hybridizing with mRNA encoding NLRP3. RNA inhibition may be achieved by blocking translation of targeted mRNA or blocking functional regions of non-coding RNA. RNA inhibition may be achieved by signaling for RNase degradation of the target RNA. In some embodiments, a nanoligomer may include a targeting sequence capable of hybridizing with DNA encoding NLRP3. In some embodiments, a nanoligomer may be capable of downregulating expression of the NF-κβ gene. In some embodiments, a nanoligomer may include a targeting sequence capable of hybridizing with a polynucleotide encoding NF-κβ. In some embodiments, a nanoligomer may include a targeting sequence capable of hybridizing with mRNA encoding NF-κβ. RNA inhibition may be achieved by blocking translation of targeted mRNA or blocking functional regions of non-coding RNA. RNA inhibition may be achieved by signaling for RNase degradation of the target RNA. In some embodiments, a nanoligomer may include a targeting sequence capable of hybridizing with DNA encoding NF-κβ. In some embodiments, a nanoligomer may include a targeting sequence capable of hybridizing with a polynucleotide encoding a gene selected from IL-1β, IL-1α, TNF-α, IL-6, IL-4, IL-13, AIM2, TNRF1, NLRP1, NLRP6, NLRC4, NLRP3, and NF-κβ.

Still referring to FIG. 1, in some embodiments, a composition comprising a nanoligomer targeting NLRP3 and a nanoligomer targeting NF-κβ is administered to a subject. In some embodiments, administration of a NLRP3 nanoligomer and a NF-κβ nanoligomer may lead to a reduction in IL-18 levels compared to a nanoligomer targeting only one of them or compared to a control. In some embodiments, administration of a NLRP3 nanoligomer and a NF-κβ nanoligomer may lead to a reduction in IL-1 levels compared to a nanoligomer targeting only one of them or compared to a control. In some embodiments, administration of a NLRP3 nanoligomer and a NF-κβ nanoligomer may lead to a reduction in IL-4 levels compared to a nanoligomer targeting only one of them or compared to a control. In some embodiments, administration of a NLRP3 nanoligomer and a NF-κβ nanoligomer may lead to a reduction in CD30 levels compared to a nanoligomer targeting only one of them or compared to a control. In some embodiments, administration of a NLRP3 nanoligomer and a NF-κβ nanoligomer may lead to a reduction in IL-31 levels compared to a nanoligomer targeting only one of them or compared to a control. In some embodiments, administration of a NLRP3 nanoligomer and a NF-κβ nanoligomer may lead to a reduction in CXCL5 levels compared to a nanoligomer targeting only one of them or compared to a control. In some embodiments, administration of a NLRP3 nanoligomer and a NF-κβ nanoligomer may lead to a reduction in CCL4 levels compared to a nanoligomer targeting only one of them or compared to a control. In some embodiments, administration of a NLRP3 nanoligomer and a NF-κβ nanoligomer may lead to a reduction in CCL20 levels compared to a nanoligomer targeting only one of them or compared to a control. In some embodiments, administration of a NLRP3 nanoligomer and a NF-κβ nanoligomer may lead to a reduction in CXCL11 levels compared to a nanoligomer targeting only one of them or compared to a control. In some embodiments, administration of a NLRP3 nanoligomer and a NF-κβ nanoligomer may lead to a reduction in CD4OL levels compared to a nanoligomer targeting only one of them or compared to a control.

Still referring to FIG. 1, in some embodiments, one or more nanoligomers are formulated with a pharmaceutically acceptable excipient. As used herein, a “pharmaceutically acceptable excipient” is a pharmaceutically acceptable material, composition or vehicle involved in carrying or transporting a payload from one cell-type, organ, or portion of the body to another cell-type, organ, or portion of the body. Pharmaceutically acceptable excipients include, for example, solvents, dispersion media, coatings, antibacterial and antifungal agents, isotonic and absorption delaying agents, liquid or solid fillers, diluents, excipients, manufacturing aids (such as lubricants, talc magnesium, calcium or zinc stearate, or steric acid), or solvent encapsulating materials. Each pharmaceutically acceptable excipient may be “acceptable” in the sense of being compatible with the other ingredients of the formulation and not injurious to the subject. Some examples of materials which may serve as pharmaceutically-acceptable excipients include, without limitation: (1) sugars, for example lactose, glucose, mannose and/or sucrose; (2) starches, for example corn starch and/or potato starch; (3) cellulose, and its derivatives, for example sodium carboxymethyl cellulose, methylcellulose, ethyl cellulose, microcrystalline cellulose and/or cellulose acetate; (4) powdered tragacanth; (5) malt; (6) gelatin; (7) lubricating agents, for example magnesium stearate, sodium lauryl sulfate and/or talc; (S) excipients, for example cocoa butter and/or suppository waxes; (9) oils, for example peanut oil, cottonseed oil, safflower oil, sesame oil, olive oil, corn oil and/or soybean oil; (10) glycols, for example propylene glycol; (11) polyols, for example glycerin, sorbitol, and/or mannitol; (12) esters, for example glycerides, ethyl oleate and/or ethyl laurate; (13) agar; (14) buffering agents, for example magnesium hydroxide and/or aluminum hydroxide; (15) alginic acid; (16) pyrogen-free water; (17) diluents, for example isotonic saline, and/or PEG400; (18) Ringer's solution; (19) C2-C12 alcohols, for example ethanol; (20) fatty acids; (21) pH buffered solutions; (22) bulking agents, for example polypeptides and/or amino acids (23) serum component, for example serum albumin, HDL and LDL; (24) surfactants, for example polysorbates (Tween 80) and/or poloxamers; and/or (25) other non-toxic compatible substances employed in pharmaceutical formulations: for example, fillers, binders, wetting agents, coloring agents, release agents, coating agents, sweetening agents, flavoring agents, perfuming agents, preservatives and/or antioxidants.

Still referring to FIG. 1, a composition described herein may be administered to a subject by any one of a variety of manners or a combination of varieties of manners. For example, a composition may be administered orally, nasally, intraperitoneally, or parenterally, by intravenous, intramuscular, topical, or subcutaneous routes, or by injection into tissue.

Still referring to FIG. 1, a composition described herein may be administered to a subject that has a disease, or that has a risk of developing a disease. As an example, a composition described herein may be administered to a subject that has a disease associated with neuroinflammation. A composition described herein may be administered to a subject that has a disease selected from the list IBD, Alzheimer's disease, Parkinson's disease, multiple sclerosis, prion's disease/Creutzfeldt-Jakob disease (CJD), neurodegenerative diseases, autoimmune diseases, cancer, liver fibrosis, NASH, diabetes, gout, myocardial infarction, and sepsis. In some embodiments, a disease may be treated via a method including administering to a subject one or more nanoligomers, or a composition including one or more nanoligomers. For example, a disease may be treated by administering a composition comprising a nanoligomer targeting NF-κβ DNA or mRNA, and a nanoligomer targeting NLRP3 DNA or mRNA. As used herein, “treating” or “treatment” means the treatment of a disease or condition of interest in a subject having the disease or condition of interest, and includes: (i) preventing the disease or condition from occurring in the subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition, i.e., relieving pain without addressing the underlying disease or condition.

Still referring to FIG. 1, in some embodiments, a composition described herein may be administered to a subject that has inflammatory bowel disease (IBD). In some embodiments, a composition described herein may be administered to a subject that has a risk of developing IBD. In some embodiments, IBD may refer to Chron's disease and ulcerative colitis. In some embodiments, IBD may be characterized by chronic inflammation of the GI tract. In some embodiments, a composition described herein may be administered to a subject that has Chron's disease. In some embodiments, a composition described herein may be administered to a subject that has ulcerative colitis.

Still referring to FIG. 1, in some embodiments, a therapeutically effective amount of a composition, such as a composition including one or more nanoligomers, is administered to a subject. As used herein, an “effective amount” or “therapeutically effective amount” is the amount of a composition of this disclosure which, when administered to a subject, is sufficient to effect treatment of a disease or condition in the subject. The amount of a composition of this disclosure which constitutes a “therapeutically effective amount” will vary depending on the composition, the condition and its severity, the manner of administration, and the age of the subject to be treated. As used herein, a “subject” is a mammal that has a disease or condition of interest, or that has a risk of developing a disease or condition of interest. In some embodiments, a subject is a homo sapiens.

Neuroprotection using an immunotherapeutic nanoligomer in prion diseased mice could open new avenues for other neurodegenerative diseases such as but not limited to Parkinson's Disease, Prion Disease, Multiple Sclerosis, and Alzheimer's disease.

We hypothesized that downregulation of key immunotherapy targets: combination of NLRP3 inflammasome and transcription factor NF-κB, would be neuroprotective. To test this, we utilized Nanoligomers in a prion-diseased mouse to assess the impact on glial inflammation, behavioral/cognition deficits, aggregation of misfolded proteins, neuroinflammatory signaling, and loss of neurons. The treatment showed decreased numbers of microglia and S100β positive astrocytes, markers of neuroinflammation, and improved hippocampal behaviors and cognitive tests, indicating slowed disease pathogenesis. Critically, the Nanoligomer protected the brain from prion-induced spongiotic change, neuronal loss, and significantly increased life span of the mice showing that Nanoligomer inhibited key inflammatory pathways can prevent neuronal death and slow the progression of neurodegenerative diseases.

Neurogenerative diseases are on the rise impacting millions of people worldwide. These diseases are commonly characterized by an increase in glial inflammation or activation and aggregation of misfolded proteins that increase over time, followed by irreversible neuronal loss. Importantly there are no effective therapies that halt this disease progression allowing for neuronal protection. There are many laboratory models commonly utilized for the studying of these diseases, however few rodent models have all aspects of human neurodegenerative protein-misfolding diseases (NPMDs). Most transgenic mouse models for Alzheimer's disease (AD), AD related diseases (ADRDs) or Parkinson's diseases display few of the neuronal pathogenesis, behavioral and cognitive deficits or clinical sign phenotypes. To study therapeutic interventions, we used a wild-type mouse that actually develops a NPMD disease following inoculation with prions. Prion diseases are rare neurodegenerative diseases that undergo the common characteristics of NPMDs as the disease progresses. Previous literature has shown that therapeutic developed for prion diseased mice can be translated to other NPMDs (cite). While a few compounds have been shown to reduce signs of prion disease in mouse models, these compounds have toxic effects in the brain or elsewhere.

Prion diseases result from the native conformation of the cellular prion protein (PrPC) misfolding to the infectious form, PrPSc. This misfolding occurs when the alpha helical PrPC protein is conformationally changed to the β-sheet rich PrPSc, which forms amyloid fibrils and aggregates, causing the disruption of brain homeostasis. Similar to other NPMDs an early sign of prion disease, thought to be caused by the aggregation of PrPSc, is toxic glial inflammation with activation of microglia and astrocytes and systemic inflammation. However, it may be that PrPSc itself is not neurotoxic, and other cellular stress pathways, including glial inflammation, play a major role in disease pathogenesis. Neuroinflammation results in increased oxidative stress, secretion of cytokines, disruption of neural signaling, and glial scarring leading to the subsequent damage and loss of synapses, neuronal dysfunction and neuronal death. Chronic neuroinflammation is known to be damaging to nervous tissue and contributes to the development of prion diseases and other NPMDs.

To identify the optimal targets for tackling neuroinflammation cascade, we screened the downregulation of several proinflammatory cytokines (e.g., Interleukin-1β or IL-1β, tumor necrosis factor-alpha or TNF-α, Interleukin 6 or IL-6), inflammasomes (e.g., NLRP3, NLRP1), key transcription factors (e.g., nuclear factor kappa-B or NF-κβ), and their combinations. Using administration of non-toxic, targeted, effective, and bioavailable Nanoligomers that can be administered as naked molecules through injection or inhalation and designed to cross the blood-brain-barrier, we identified a combination of NLRP3 inflammasome and NF-κβ transcription factor as a viable candidate. During brain stress, like NPMDs, there is significant cross-talk between microglia and astrocytes which involves critical inflammatory cell signaling events, like NF-κβ and the inflammasome formation. The transcription factor, NF-κβ, when translocated into the nucleus causes a number of inflammatory cytokines and chemokines to be transcribed causing a cascade of microglia and astrocyte inflammation. Another cell signaling pathway shown to be critical for NPMDs, like prion diseases, is the activation of the inflammasome, specifically caused by the NLRP3 protein. We hypothesized that if we can inhibit both of these pathways simultaneously that prion disease and other neurodegenerative diseases such as Parkinson's Disease, Prion Disease, Multiple Sclerosis, and Alzheimer's disease, progression will slow, as a proof of principle for other NPMDs. Critically in this study, these Nanoligomers were able to save neurons, reduce spongiotic change, decrease behavioral and cognitive deficits and glial inflammation in prion diseased brains once the infection had already been ensued.

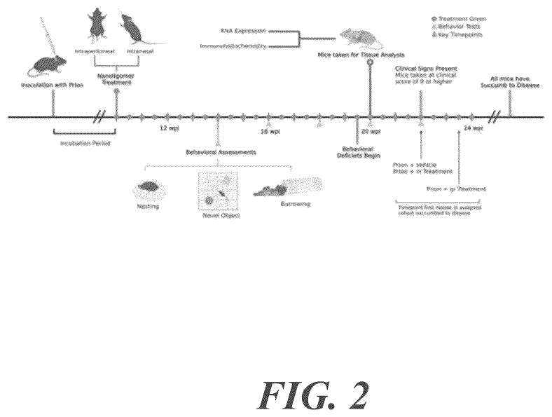

Protection of the Nanoligomer against prion-induced cognitive and behavioral deficits. Wild-type, C57Bl6/J, (Jackson laboratories) mice were infected intracerebrally with brain homogenates from Rocky Mountain Laboratories (RML) prions or normal brain homogenates (NBH) as the negative control. Nanoligomer treatment began at 10 weeks post inoculation (wpi) or approximately 40% of full disease progression and continued until the mice succumbed to prion disease. 10 mg/Kg of the Nanoligomer SB_NI_112 or saline as the vehicle, was given either intraperitoneally (i.p.) or intranasally (i.n.) twice a week to both prion and NBH mice, as shown in FIG. 1. As the disease progressed and treatment continued behavioral assessments including novel object recognition, burrowing, and nesting, were performed at 12 wpi to train and establish a baseline for each experimental cohort, shown in FIG. 2. All three behavioral assessments measure hippocampal integrity, the brain region known to show the first clinical pathology including glial inflammation and spongiotic change.

Mice with RML induced prion disease have a significant decrease in the ability to recognize a familiar object as this is prion strain that begins to aggregate within the hippocampus the brain region essential for memory and learning. Both i.p. and i.n. SB_NI_112 treatment protected prion induced deficit in novel object recognition when compared to vehicle treated diseased mice at 22 wpi, shown in FIG. 2B. The ratio of time the mouse spent with the novel object to the familiar object, seen 24 hours beforehand was calculated and noted the discrimination index. The closer the discrimination index is to one the more intact the hippocampal memory is. Both intraperitoneal treated mice (p<0.0005, mean discrimination index score: 0.449) and intranasal treated mice (p<0. 005, mean discrimination index score: 0.434) had a significantly increase in novel object recognition, when compared to vehicle treated diseased mice (discrimination index of −0.174,ie. preferring the familiar object). At 20 wpi a significant rise in the mice to recognize the novel was also seen between vehicle and SB_NI_112 i.p. by analysis of the discrimination index, shown in FIG. 2A.

Two other common hippocampal specific behavioral assessments were performed on the cohorts of mice to compare prion induced deficits with and without SB_NI_112 treatment. Mice were given a plastic tube filled with food pellets within in their home cages, for 30 min to burrow freely. Following this time, the food pellets left in the tubes were weighed and percent burrowed calculated. Prion diseased mice are known to stop burrowing as disease progresses but prion diseased mouse treated with SB_NI_112 at 20 wpi, 21 wpi, and 22 wpi, did not decrease burrowing as statistically significant as vehicle only mice, shown in FIG. 2D, showing a trend of protection (detailed quantitative assessment scoring provided in Methods). Mice were given napkins to nest overnight and scored between one (no nest formed) and five (fluffy built nest) weekly starting at 16 wpi. The weights of the mice with and without treatment with SB_NI_112 were not significantly different throughout the disease progression indicating no acute toxicity due to the Nanoliogmer. Monitoring of nest formation was found to be significantly higher among i.p. treated mice at 20 wpi (p>0.005), 21 wpi (p>0.005), and 22 wpi (p>0.05), shown in FIG. 2C, compared to vehicle treated prion infected nest formation.

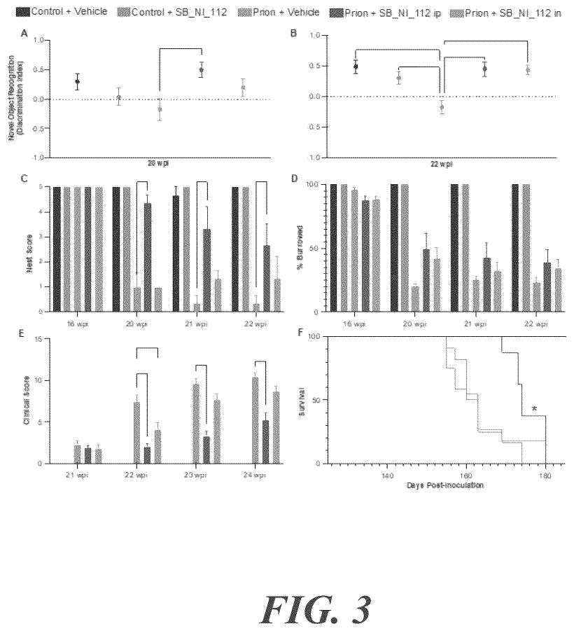

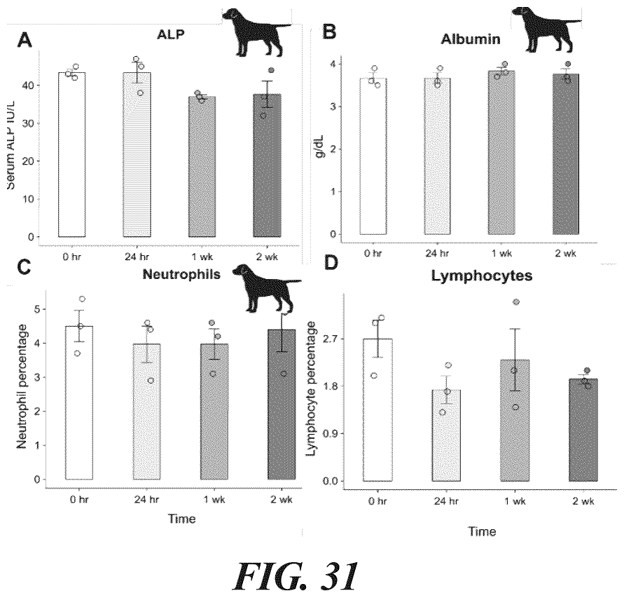

Nanoligomer SB_NI_112 treatment reduces glial inflammation. In addition to the behavioral assessments to quantify hippocampal learning deficits during disease progression, potential therapeutic effect was assessed using immunohistology and biochemical characterization. Brain tissues were collected at 20 wpi and 24 wpi from all treatment groups. Formalin fixed tissues were processed and embedded into paraffin wax. Once brains were sliced on the microtome brains were stained for both microglial and astrocytic inflammation, shown in FIG. 3. Astrocytic inflammation was identified as S100β cells, shown in FIG. 3A, and Iba1+ cells denote inflamed microglia, shown in FIG. 3F. Four brain regions known to be implemented in RML prion infection are the hippocampus, cortex, thalamus and cerebellum. In the thalamic region, both astrocytic and microglial inflammation was significantly suppressed with SB_NI_112 Nanoligomer treatment, shown in FIG. 3C and FIG. 3H, respectively, at 20 wpi. Additionally, both i.n. and i.p. SB_NI_112 treatment provided significant protection from microglia inflammation in the hippocampus, shown in FIG. 3G, cortex, shown in FIG. 31, and the cerebellum, shown in FIG. 3J.

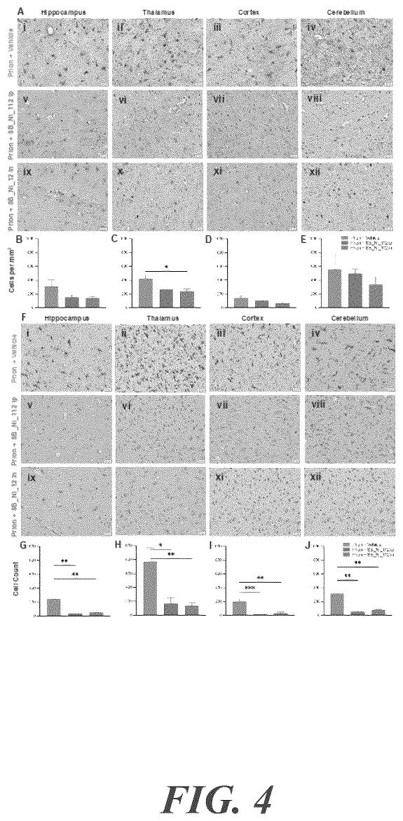

Neuronal protection using Nanoligomer SB_NI_112. A morphologic spongiotic or vacuoles within the brain tissue is a hallmark of prion disease that increases as the disease progresses. To assess and quantify this change, we used pathologic scoring of the hippocampus, thalamus, cortex, and cerebellum following H&E staining. This revealed protection against spongiotic change in both i.p. and i.n. SB_NI_112 Nanoligomer treatment of prion diseased mice, shown in FIGS. 4A and B, when compared to vehicle treated prion diseased mice. The loss of neurons within the hippocampus following RML prion infection is well established therefore we assessed this within our treatment group. Critically, neuronal numbers within the CA1 region of the hippocampus were significantly protected by SB_NI_112 Nanoligomer i.p. and i.n. treatment compared to vehicle treated (p>0.05), show in FIGS. 4C and 4D. Importantly, we see no change in the PK resistant prion protein PrP^{Sc} with Nanoligomer treatment (FIG. 4C). Therefore, the neuroprotection is independent of aggregation and misfolding of PrP^{Sc} itself and could be a good avenue of therapy for other NPMDs with differing toxic misfolded proteins.

Clinical scores and survival improved in mice treated with Nanoligomer. At 22 wpi, vehicle treated mice begin to show a significant increase clinical signs of prion disease, shown in FIG. 2E. From 22 wpi to 24 wpi i.p. and i.n. SB_NI_112 treated diseased mice have significantly (p<0.0001 and p<0.0005 respectively) lower clinical scores than vehicle treated diseased mice. Prion clinical signs included tail rigidity, hyperactivity, ataxia, extensor reflex, tremors, righting reflex, kyphosis, and poor grooming. Each sign was rated on a scale from 0-5. All clinical sign scores were combined for a total score. Mice were considered terminal and euthanized after reaching a total score of 9 or above. Date of mice succumbing to prion disease was documented for survival analysis, shown in FIG. 2E. Importantly, the lifespan of prion disease mice was significantly increased with i.p. SB_NI_112 Nanoligomer treatment (p>0.05) compared to vehicle treated mice.

The slowing of the progression of neurodegenerative protein misfolding diseases (NPMDs) has been proven to be difficult. Many promising treatments are toxic or must be delivered directly to the brain making translation to human patients difficult. Here we have successfully used a non-toxic, systemically delivered, Nanoligomer that protects against prion disease and other neurodegenerative disease, such as Parkinson's Disease, Prion Disease, Multiple Sclerosis, and Alzheimer's disease, phenotypes. Protection of cognition and other hippocampal behavioral deficits and the clinical progression of disease is accomplished with the Nanoligomer, as shown in FIG. 2. This Nanoligomer is specifically inhibiting two known inflammatory events, NF-κB and NLRP3 expression, in the brain during these diseases and significantly decreases microglial and astrocytic inflammation, slowing disease progression for prion disease and other neurodegenerative disease, such as Parkinson's Disease, Prion Disease, Multiple Sclerosis, and Alzheimer's disease. For instance, it may be slowing disease progression through the inhibition of glial inflammation, neurons were protected shown by a decrease in spongiotic change and neuronal loss, independent of a decrease in the misfolded protein itself. Critically, these findings show that these Nanoligomers can slow both early cognitive/behavioral phenotypic changes and later clinical signs, neuronal loss and death. Critically, we have identified a treatment for a neurodegenerative disease that is non-toxic and can be delivered to the periphery. These findings of neuroprotection within a prion diseased mouse can be easily translated to other neurodegenerative disease as neuroinflammation is a generic pathology irrespective of the misfolded protein.

Referring now to FIG. 2, an exemplary schematic of experimental set-up is shown.

Still referring to FIG. 2, prion diseased mice were treated with Nanoligomers or vehicle through intraperitoneal or intranasal routes of exposure, twice a week starting at 10 weeks post inoculation (10 wpi). Throughout the progression of the disease cognitive, hippocampal specific behaviors and clinical signs were monitored. At 20 wpi brain tissue was dissected for analysis of disease progression including neuroinflammation, spongiotic change and neuronal loss.

Referring now to FIG. 3, an exemplary embodiment of cognition and behavioral deficits that are protected by Nanoligomers treated by intraperitoneal route is shown.

Still referring to FIG. 3, mice were monitored for changes in cognition and behavior throughout the disease progression. Nanoligomer treatment (SB_NI_112) protected the prion induced deficit in spatial novel object recognition seen in the vehicle treated prion diseased mice when given intraperitoneal (i.p.) for 20 wpi, as shown in A, and i.p and intranasal (i.n) at 24 wpid, as shown in B. As shown in C, no significant change was seen in the hippocampal assay burrowing when mice were treated with the nanoligomers compared to vehicle. Further, as shown in D, a significant increase in the ability of the mice to build proper nests at 20 and 21 wpi with i.p. treatment of the nanoligomers compared to vehicle treatments. As shown in E, prion clinical scores were monitored and shown to be protected by both intranasal and i.p. treatment at 22 wpi and continued to protective with i.p. until 25 wpi. Additionally, as shown in F, the life-span of these prion diseased mice was significantly elongated with i.p. nanoligomer treatment. N=9 for prion positive i.p. and i.n. groups, N=12 for all other groups. One-way ANOVA and post-hoc Tukey test, error bars=SEM, *p<0.05, **p<0.01, ***p<0.001, ****p<0.0001, ns=not significant.

Referring now to FIG. 4, an exemplary embodiment of microglia and astrocytic inflammation that are significantly reduced using Nanoligomers in prion diseased mice is shown.

Still referring to FIG. 4, Section A shows exemplary representative images of the hippocampus (i, v, ix), thalamus (ii, vi, x), cortex (iii, vii, xi) and cerebellum (iv, viii, xii) brain regions of S100b+ cells, a maker of inflamed astrocytes, with SB_NI_112 intraperitoneal (i.p.) and intranasal (i.n.) treatment. Quantitative analysis of each brain region identifies a significant change in the thalamus, shown in graph C, and a trend of a decrease of inflamed astrocytes in the hippocampus, shown in graph B, and cortex, as shown in D, and cerebellum, as shown in E, with treatment. Section F shows exemplary representative images of the hippocampus (i, v, ix), thalamus (ii, vi, x), cortex (iii, vii, xi) and cerebellum (iv, viii, xii) brain regions for IBA1+ cells, a maker for microglia, with and without treatment. Quantitative analysis of each brain region identifies a significant change in the hippocampus, as shown in G, thalamus, as shown in H, cortex, as shown in I, and cerebellum, as shown in J, with both i.n. and i.p. treatment. Arrows represent examples of positive cells. Scale bar=20 μm N=3-4. One-way ANOVA *p>0.05, **p>0.001, ***p>0.0001

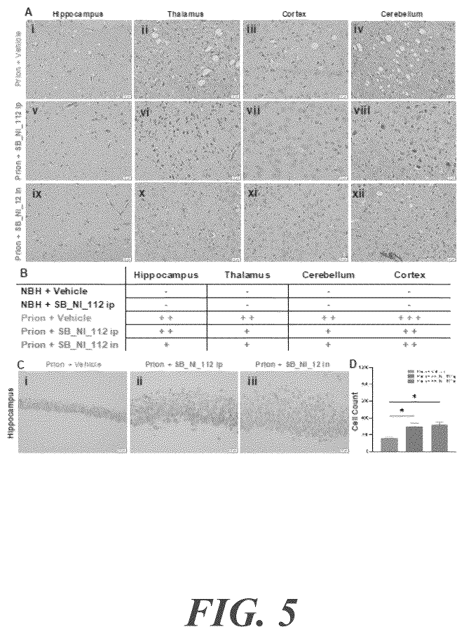

Referring now to FIG. 5, an exemplary embodiment of Prion induced spongiotic change and neuronal loss is significantly decreased with Nanoligomer SB_NI_112 treatment is shown.

Still referring to FIG. 5, Section A shows Representative images of the spongiotic change in hippocampus (i, v, ix), thalamus (ii, vi, x), cortex (iii, vii, xi) and cerebellum (iv, viii, xii) brain regions were protected with SB_NI_112 intraperitoneal (i.p.) and intranasal (i.n.) treatment. Section B shows exemplary Pathological scoring of each brain region identifies a protection or decrease of pathological score at all brain regions analyzed. Arrows point to spongiotic change. Section C shows exemplary representative images of the NeuN+cells (neuronal cell bodies) in the CA1 region of the hippocampus. Section D shows exemplary significant decrease in the number of neurons lost at 20 wpi with treatment via i.p. and i.n. SB_NI_112. Scale bar=20 mm N=3-4. One-way ANOVA *p>0.05.

### Materials and Methods

### Ethics Approval

Mice were euthanized by deeply anaesthetizing with isoflurane followed by decapitation. All mice were bred and maintained at Lab Animal Resources, accredited by the Association for Assessment and Accreditation of Lab Animal Care International, in accordance with protocols approved by the Institutional Animal Care and Use Committee at Colorado State University.

### Nanoligomer Development

Nanoligomers were synthesized using previously published procedure. Briefly, the Sachi bioinformatics platform was used to identify the lead Nanoligomer sequence of the DNA/RNA binding domain (DBD/RBD). The desired peptide molecules were synthesized using solid phase peptide synthesis, and conjugated to the gold nanoparticle, for high-throughput purification and delivery. The concentration of the peptide molecules and Nanoligomers was quantified using ultraviolet-visible (UV-Vis) optical spectroscopy.

### Mice and Brain Homogenates

C57Bl/6 (Jackson Laboratory) mice were intracranially inoculated with 30 μl of 1% 22L or Rocky Mountain Laboratories (RML) strains of mouse-adapted prions, or normal brain homogenate (NBH). Mice were monitored for weight loss and clinical signs of prion disease and euthanized after showing signs of terminal illness. 20% brain homogenates in phosphate-buffered saline (PBS) were made using beads and a tissue homogenizer (Benchmark Bead Blaster 24) and stored at −80C. Brain homogenates were aliquoted and treated with UV light for 30 minutes to sterilize before being used for cell culture.

Nanoligomer Administration. At 10 weeks post RML prion or NBH inoculation, mice were treated two times per week either intraperitoneally (i.p.) or intranasally (i.n.) with 10 mg/kg of Nanoligomer SB_NI_112 diluted in sterile saline. Mice receiving treatment intranasally were anesthetized prior to administration and laid in an intranasal apparatus that controlled isoflurane throughout the intranasal procedure.

### Cognitive Assay: Novel Object Recognition

Mice were tested in a rectangular arena (28 cm×43 cm). Mice were habituated to the arena seven days before testing. On day one of habituation, mice were permitted to explore the empty arena for ten minutes. On day two of habituation, two identical objects (constructed with Legos) were placed in the arena, approximately 10 cm from the walls with 20 cm distance between. Each mouse was placed in the arena for ten minutes of exploration. During day one of testing, mice were placed in the arena with two identical objects and were given 5 minutes of exploration. On day two of testing, one of the known objects was replaced by a novel object, and the 5-minute exploration was filmed using a 1080 P FHD Mini Video Camera. All objects and the arena were cleansed thoroughly between trials to ensure the absence of olfactory cues. Time exploring both the known and novel object was calculated blinded. Discrimination index calculated following Absolute vs Relative Analysis protocol.

### Hippocampal Behavioral Assays: Burrowing and Nesting

Briefly, mice were placed in a large cage with a PVC tube full of food pellets, as described (23). The natural tendency of rodents is to displace (burrow) the food pellets. The percentage of burrowing activity is calculated from the difference in the weight of pellets in the tube before and after 2 hours. To test nesting we used three fresh napkins were placed in each cage. After 24 hours, cages were examined for nesting activity. Nests were scored on a scale of 0-5, where 0 represents no nesting activity, and 5 represents a high-quality nest.

### Clinical Scoring of Mice

Eight key clinical signs were monitored in mice daily beginning at 20 wpi. Clinical signs included tail rigidity, hyperactivity, ataxia, extensor reflex, tremors, righting reflex, kyphosis, and poor grooming. Each sign was rated on a scale from 0-5. All clinical sign scores were combined for a total score. Mice considered terminal and euthanized after reaching a total score of 9 or above.

### Immunohistochemistry

Paraffin-embedded brains were sectioned at 4 um and stained with NeuN antibody (1:250; Cell Signaling) for CA1 neuronal counts. Astrogliosis was detected with Ibal antibody (1:400; Abcam). S100B antibody (AbCam; 1:400) was used for microglial counts. Nonspecific binding was blocked before primary antibodies with 10% Horse Serum (Vector Labs). A biotinylated secondary antibody (Vector Labs) was used, and stain was visualized with diaminobenzidine reagent. All images were taken with *** software and counted with CellSens (Ibal and S100B) or manually (NeuN).

### Reverse Transcriptase Quantitative PCR Analysis

RNA was extracted from cell culture 6-well dishes using cell scraping, QlAshredder and RNeasy extraction kits, in accordance with manufacturer's protocol, including a DNase digestion step with the RNase free DNase kit (Qiagen, Valencia, CA). Purity and concentration were determined using a ND-1000 spectrophotometer (NanoDrop Technologies, Wilmington, DE). Following isolation and purification, 25 ng of RNA was reverse transcribed using the iScript Reverse Transcriptase kit (BioRad, Hercules CA). The cDNA was amplified within 24 hours of reverse transcription using iQ SYBR Green Supermix (BioRad, Hercules CA). The corresponding validated primer sequences were used for each gene at 10 M. The expression data was analyzed using the 2-ΔΔCT method and normalized to expression of reference genes β-actin . The fold difference was compared to control (normal brain homogenate treated) samples (27). Validated primer sequences are as follows:

(β-actin) 5′-CCACTGTCGAGTCGCGT -3′ (forward), 5′-CGCAGCGATATCGTCATCCAT -3′ (reverse); (NLRP3) 5′-CCTGGGGGACTTTGGAATCA-3′ (forward), 5′-GACAACACGCGGATGTGAGA -3′ (reverse); (IL1β) 5′-GCAGCAGCACATCAACAAG-3′ (forward), 5′-CACGGGAAAGACACAGGTAG-3′ (reverse); (NF-κB1) 5′-GTGGAGGCATGTTCGGTAGT-3′ (forward), 5′-CCTGCGTTGGATTTCGTGAC-3′ (reverse); (TNFR1a) 5′-GTTGTCAATTGCTGCCCTGTC-3′ (forward), 5′-CAGTGACCCCTGATGGATGT-3′ (reverse). All RT-PCR was done following MIQE guidelines.

### Immunoblotting

Brain homogenates were isolated using phosphate buffered saline (PBS) supplemented with Phos-STOP and Complete protease inhibitors (Roche). A BCA Protein Assay kit (Thermo Scientific) was used to quantify protein concentration of lysates, and 25 μg of protein was digested with 20 μg/mL proteinase K (PK) (Roche) for PrPSc blots for 1 hour at 37C. Digestion was terminated with 2 mM PMSF. For PrPC blots, 10 μg of samples was used. Samples were run using 4-20% acrylamide SDS page gels (BioRad) and then transferred onto PVDF blotting paper (MilliPore). Primary antibody Bar-224 (Cayman Chemical) was used at 1:5,000 in 5% non-fat milk in tris buffered saline with tween and HRP-conjugated secondary antibodies at 1:5,000 (Vector Laboratories). The protein antibody complex was visualized using SuperSignal West Pico PLUS Chemiluminescent Substrate (Thermo Scientific) and visualized with the BioRad ChemiDoc MP.

In some embodiments, AI or machine learning may be used to select one or more target genes. In some embodiments, AI or machine learning may be used to select one or more nanoligomers.

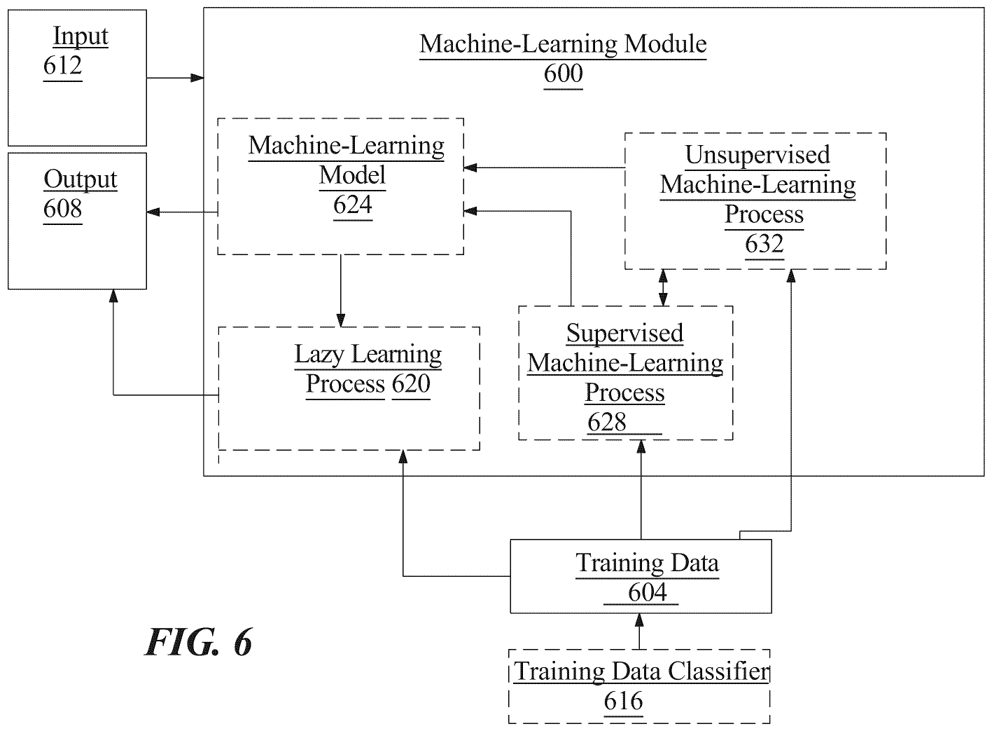

Referring now to FIG. 6, an exemplary embodiment of a machine-learning module 600 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 604 to generate an algorithm that will be performed by a computing device/module to produce outputs 608 given data provided as inputs 612; this is in contrast to a non-machine learning software program where the commands to be executed are determined in advance by a user and written in a programming language.

Still referring to FIG. 6, “training data,” as used herein, is data containing correlations that a machine-learning process may use to model relationships between two or more categories of data elements. For instance, and without limitation, training data 604 may include a plurality of data entries, each entry representing a set of data elements that were recorded, received, and/or generated together; data elements may be correlated by shared existence in a given data entry, by proximity in a given data entry, or the like. Multiple data entries in training data 604 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 604 according to various correlations; correlations may indicate causative and/or predictive links between categories of data elements, which may be modeled as relationships such as mathematical relationships by machine-learning processes as described in further detail below. Training data 604 may be formatted and/or organized by categories of data elements, for instance by associating data elements with one or more descriptors corresponding to categories of data elements. As a non-limiting example, training data 604 may include data entered in standardized forms by persons or processes, such that entry of a given data element in a given field in a form may be mapped to one or more descriptors of categories. Elements in training data 604 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively, or additionally, and continuing to refer to FIG. 6, training data 604 may include one or more elements that are not categorized; that is, training data 604 may not be formatted or contain descriptors for some elements of data. Machine-learning algorithms and/or other processes may sort training data 604 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 604 to be made applicable for two or more distinct machine-learning algorithms as described in further detail below. Training data 604 used by machine-learning module 600 may correlate any input data as described in this disclosure to any output data as described in this disclosure.

Further referring to FIG. 6, training data may be filtered, sorted, and/or selected using one or more supervised and/or unsupervised machine-learning processes and/or models as described in further detail below; such models may include without limitation a training data classifier 616. Training data classifier 616 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 600 may generate a classifier using a classification algorithm, defined as a processes whereby a computing device and/or any module and/or component operating thereon derives a classifier from training data 604. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to utilize potential match/mismatch of each candidate along the human genome.

Still referring to FIG. 6, machine-learning module 600 may be configured to perform a lazy-learning process 620 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 604. Heuristic may include selecting some number of highest-ranking associations and/or training data 604 elements. Lazy learning may implement any suitable lazy learning algorithm, including without limitation a K-nearest neighbors algorithm, a lazy naive Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 6, machine-learning processes as described in this disclosure may be used to generate machine-learning models 624. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 624 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 624 may be generated by creating an artificial neural network, such as a convolutional neural network comprising an input layer of nodes, one or more intermediate layers, and an output layer of nodes. Connections between nodes may be created via the process of “training” the network, in which elements from a training data 604 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 6, machine-learning algorithms may include at least a supervised machine-learning process 628. At least a supervised machine-learning process 628, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include inputs as described above as inputs, outputs as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 604. Persons skilled in the art, upon reviewing the entirety of this disclosure, will be aware of various possible variations of at least a supervised machine-learning process 628 that may be used to determine relation between inputs and outputs. Supervised machine-learning processes may include classification algorithms as defined above.

Further referring to FIG. 6, machine learning processes may include at least an unsupervised machine-learning processes 632. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 6, machine-learning module 600 may be designed and configured to create a machine-learning model 624 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 6, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naive Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

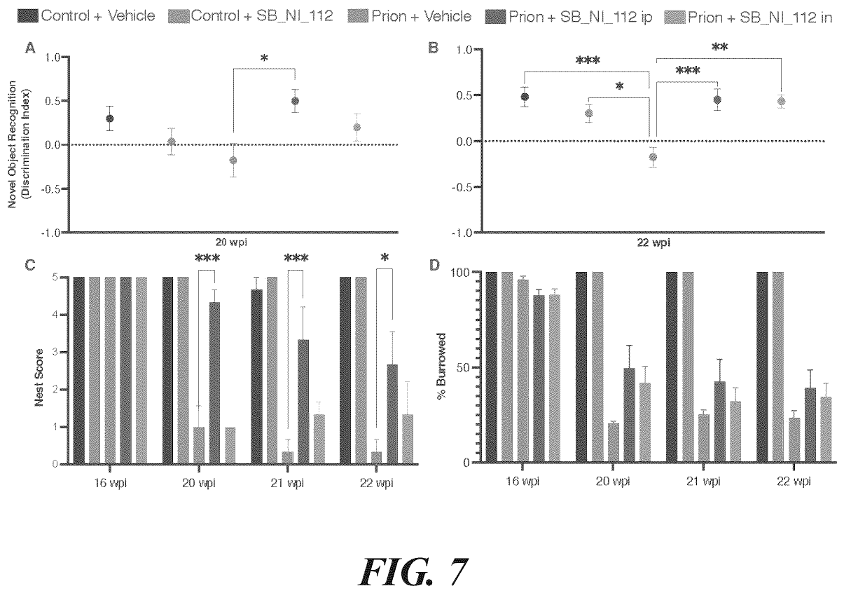

Now referring to FIG. 7, results of a prion disease mouse study are depicted. Wild type C57B16/J (prion disease model) mice were administered control+vehicle, control+SB_NI_112, prion+vehicle, prion+SB_NI_112 (intraperitoneal), or prion+SB_NI_112 (intranasal). Normal brain homogenates was used as the negative control. A and B: the ability of mice to recognize a familiar object was measured based on the ratio of time a mouse spent with a novel object to a familiar object seen 24 hours beforehand. A: intraperitoneal, 20 weeks post inoculation (wpi). B: intraperitoneal and intranasal, 24 wpi. C: Starting at 16 wpi, mice were given napkins to nest overnight and scored between 1 (no nest formed) and 5 (fluffy nest). D: Burrowing was also monitored: mice were placed in a cage with a plastic tube containing food pellets. After 30min, the percent of pellets still in the tube was weighed. Error bars=SEM, *p<0.05, **p<0.01, ***p<0.001, ****p<0.0001, ns=not significant.

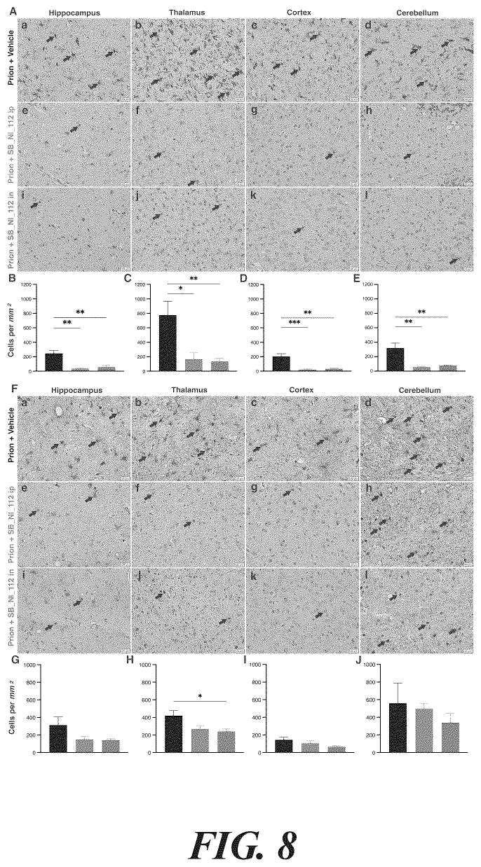

Now referring to FIG. 8, results of another prion disease mouse study are depicted. A: representative images of hippocampus, thalamus, cortex and cerebellum brain regions of S100β+ cells, a maker of inflamed astrocytes, with SB_NI_112 intraperitoneal and intranasal treatment is depicted. Cells per mm\hat{ }2 in the hippocampus (B), thalamus (C), cortex (D), and cerebellum (E) were measured. F: representative images of hippocampus, thalamus, cortex and cerebellum brain regions of IBA1+ cells, a maker for microglia with and without treatment was measured. Cells per mmA2 in the hippocampus (G), thalamus (H), cortex (I), and cerebellum (J) were measured. *p>0.05, **p>0.001, ***p>0.0001

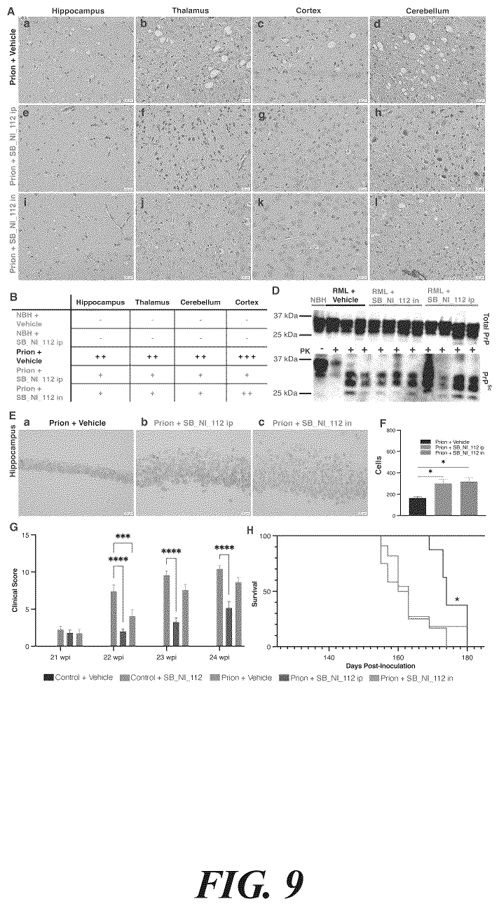

Now referring to FIG. 9, results of another prion disease mouse study are depicted. A: representative images of hippocampus, thalamus, cortex and cerebellum brain regions of mice given intraperitoneal and intranasal SB_NI_112 treatment is depicted. B: pathological scoring of each brain region. D: Representative images of the NeuN+cells (neuronal cell bodies) in the CA1 region of the hippocampus. E: Neurons lost at 20 wpi with i.p. and i.n. SB_NI_112 treatment was measured. F: Prion clinical scores at 22 wpi with i.p. and i.n. SB_NI_112 treatment was measured. H: Life span of prion disease mice with varying treatments.

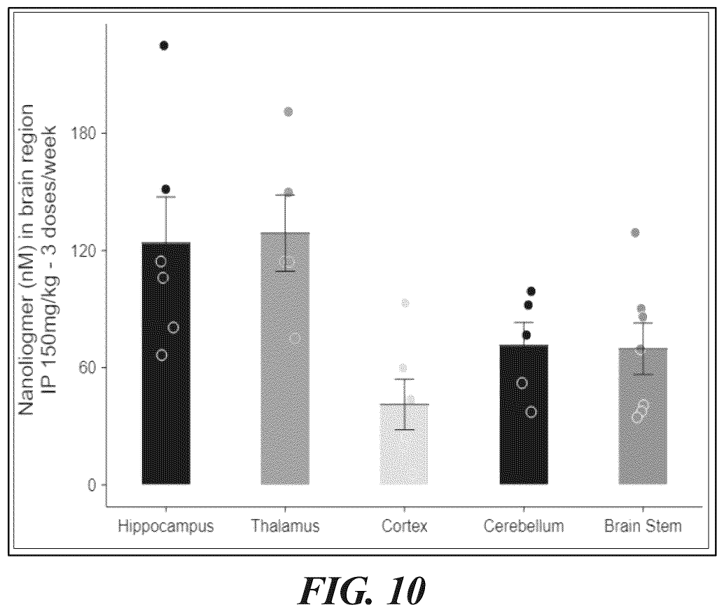

Now referring to FIG. 10, results of a study examining SB_NI_112 availability within different regions of the brain. Mice were dosed with IP administration of 150 mg/kg (maintenance dose) 3-times per week for 2 weeks. Mice were euthanized, brains were dissected using surgery to segregate different brain regions. Following organ collection, the tissues (with the Nanoligomer) were dissolved in Aqua Regia, which was then evaporated. The remaining solid was then re-suspended in nitric acid. Nanoligomer amounts were determined using Inductively Coupled Plasma Mass Spectrometry (ICP-MS).

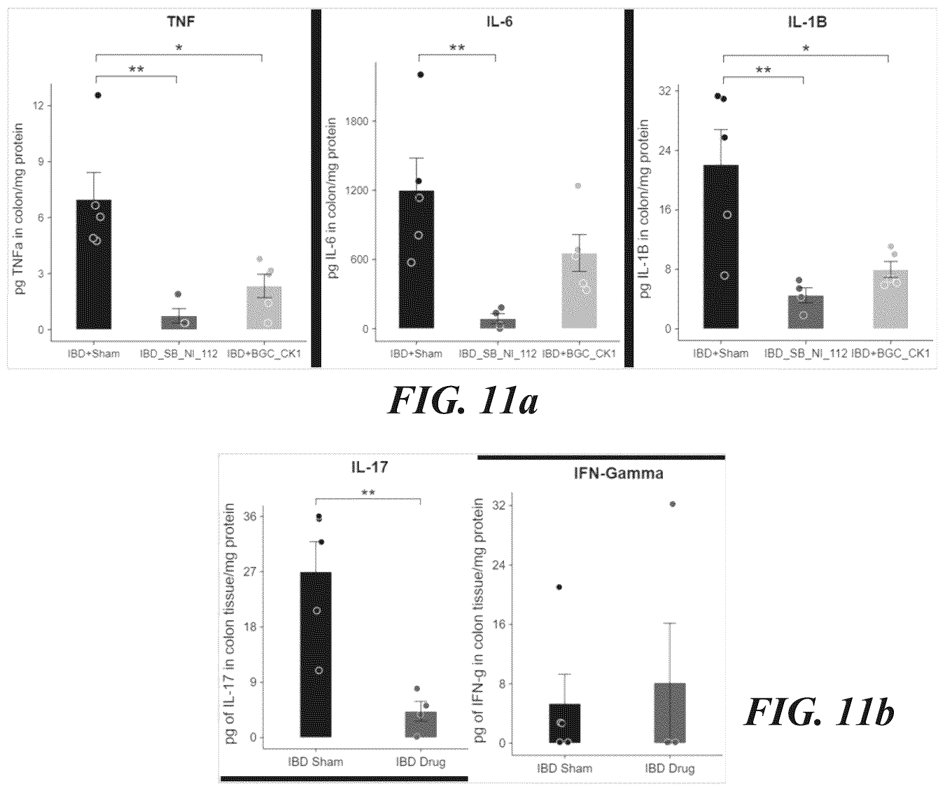

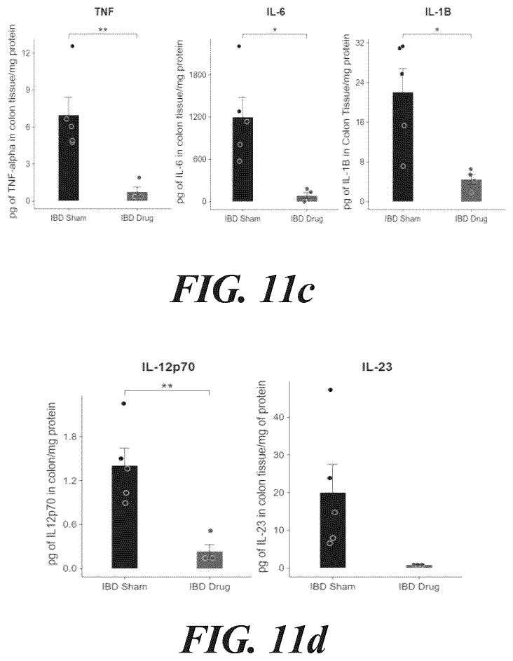

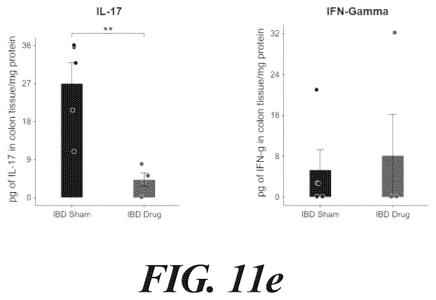

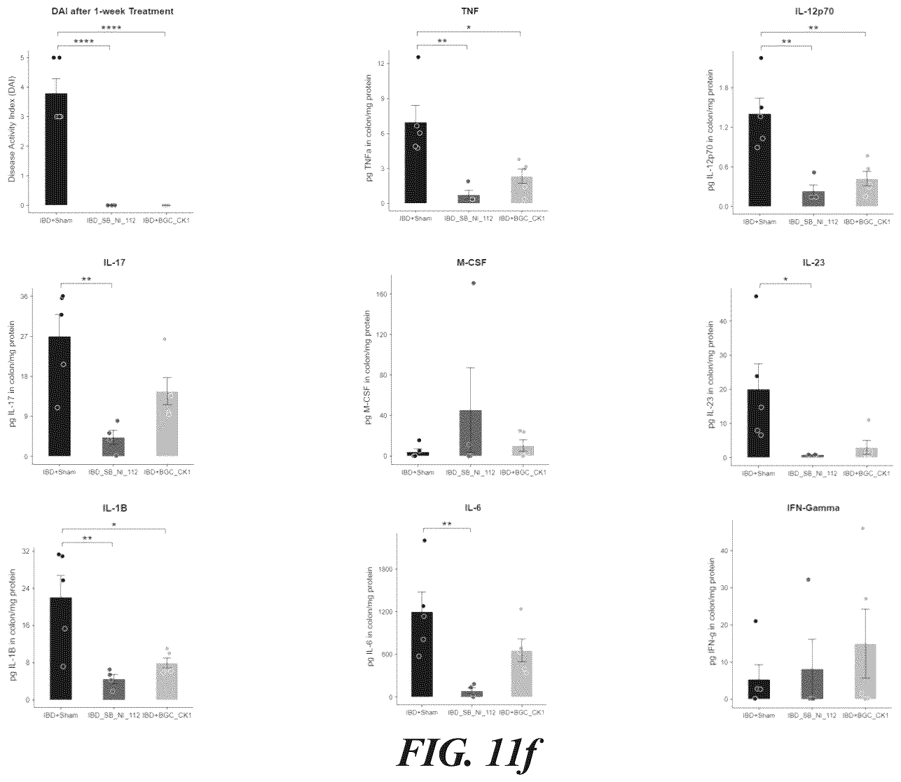

Now referring to FIG. 11, results of a study examining the role of SB_NI_112 in treating autoimmune disease such as Inflammatory Bowel Disease (IBD). Using an established Dextran Sodium Sulfate (DSS) Colitis model in mice, first C57BL-6 mice were acclimatized, their existing microbiome was wiped using 5-days of antibiotic administration, followed by recolonization with fecal gavage mixture from 10 IBD patients, to colonize mice gut with IBD-inducing microbiome. Following this, 3% DSS water was used to trigger the colitis in mice, and they were treated with either sham (saline solution) IP injection or empty (filler capsules), or treated with SB_NI_112 using IP injections or oral capsules. Following 3-dose treatment in one week, the mice colon were harvested, and analyzed for inflammation using histology and multiplexed ELISA.

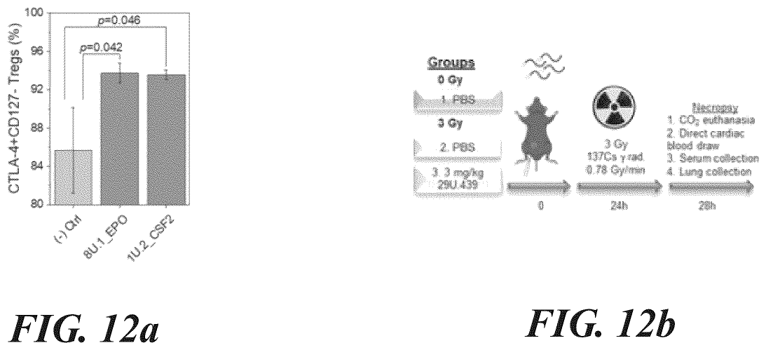

Now referring to FIG. 12, in some embodiments, a composition described herein may be administered to a subject that has an autoimmune disease or is at risk of developing an autoimmune disease. In some embodiments, a nanoligomer may regulate CSF2. While approved radioprotection therapies include Sargramostim, the impact of the inflammatory cytokine CSF2 recombinant protein on inducing further immune dysfunction especially on autoimmune diseases, including rheumatoid arthritis and multiple sclerosis has been implicated. Its potential role in astrocytosis and microgliosis leading to neurodegenerative diseases has also been revealed, leading to its use as a countermeasure target to treat radiation-induced neuropathy. However, the proposed gene-targeting to upregulate csf2 and epo resulted in upregulation of anti-inflammatory cytokines (for example IL-10) and other regulatory chemokines, pointing to controlled reversal of immune dysfunction, rather than overall increase of inflammatory markers. This also highlights the difference in immune response with recombinant protein (eg. Sargramostim) and gene therapy targeting protein upregulation.

Still referring to FIG. 12, in some embodiments, regulatory T (Treg) cells are essential for maintaining peripheral tolerance, preventing autoimmunity, and limiting chronic inflammatory diseases. To enumerate Treg cells, PBMCs were collected and stained for flow cytometry (FIG. 12B). Here we consider Tregs of relevance as the proportion of T cells that are CD25+ (Interleukin-2 receptor alpha chain) FoxP3+ (FoxP3: forkhead box P3, also known as scurfin) and their subpopulation that is CD127-(Interleukin-7 receptor alpha chain) CTLA4+ (cytotoxic T-lymphocyte-associated protein 4). To probe the impact of lead targets on Treg cell population, we conducted population-level immune regulation analysis with csf2 and epo upregulating Nanoligomers. To enumerate Treg cells, PBMCs were treated with 8U.1_EPO or 1U.2_CSF2 for 24 hours and then collected and stained for flow cytometry (FIG. 12A). We demonstrate that treatment with either 8U.1_EPO or 1U.2_CSF2 causes significant increase in Treg population compared to negative control (PBS). This observed Treg population increase highlights an additional difference in recombinant protein vs reversible gene therapy, and the potential role in regulating radiation dysfunction in multiple protein expression pathways using gene regulation networks.

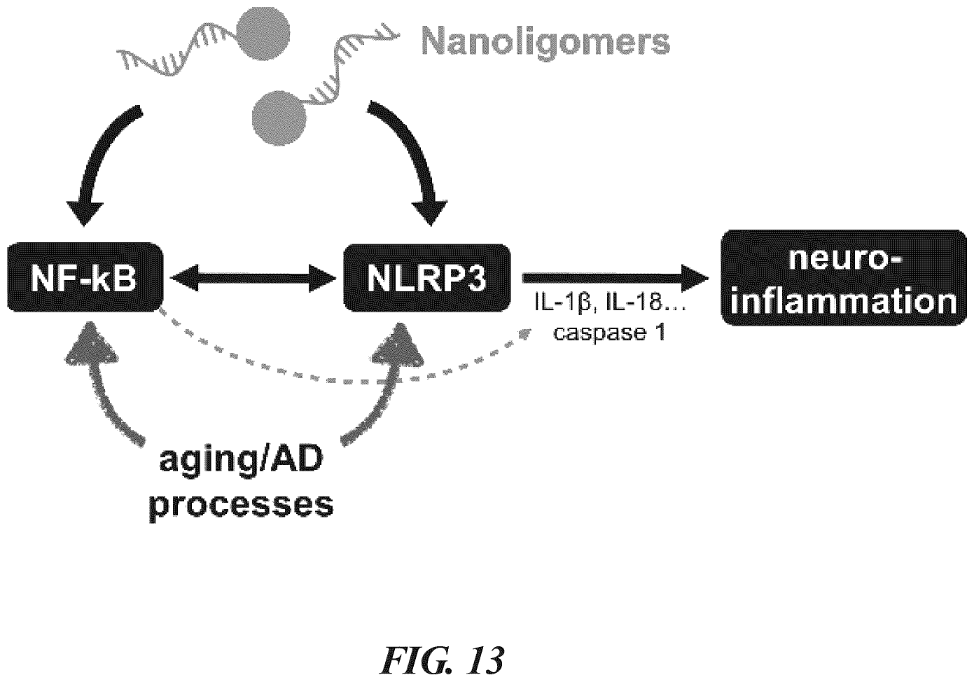

Now referring to FIG. 13, 50 million people worldwide suffer from dementia associated with aging, and ˜30 million suffer from Alzheimer's disease (AD), the most common cause of dementia. Because older age is the primary risk factor for AD, there is an important need to understand and target the mechanisms by which aging drives the development and progression of AD. Brain aging involves declines in cognitive function and pathological events that are precursors to AD (e.g., the deposition of amyloid beta (Aβ) and tau), and neuroinflammation is central contributor to all of these events. Neuroinflammation is characterized by reactive glial cells that produce pro-inflammatory, neurotoxic cytokines, and it develops with aging and in response to AD pathology. As such, identifying strategies to reduce neuroinflammation with both aging and pathology is a promising research strategy. However, generic anti-inflammatory drugs have been ineffective for treating cognitive decline/AD so it may be necessary to target specific upstream neuroinflammation mediators.

Still referring to FIG. 13, Nanoligomer immunotherapy cocktail SB_NI_112 downregulates nuclear factor kappa B (NF-κB) and NLR family pyrin domain containing 3 (NLRP3) and reduces neuroinflammation in vivo. Nanoligomers can regulate specific genes by targeting mRNA (to inhibit translation) and DNA (to increase or decrease transcription). SB_NI_112 lead molecule was developed, via bioinformatics-driven screening and testing in human brain neurospheroids to specifically target key cytokines, inflammasomes, and transcription factors involved in neuroinflammation. The brain-penetrant compound has minimal off-target effects, excellent biodistribution (including to different brain regions), requires no specific formulation and can be administered in an oral, injectable, or inhalable form. The two targets of SB_NI_112, NF-kB and NLRP3, both: 1) play a central aging and AD (e.g., in innate immune activation, cytokine production, etc.); 2) impair cognitive function in transgenic mouse models; 3) increase AD risk; and 4) exacerbate pathology in response Aβ and tau. However, no one has specifically targeted both NF-kB and NLRP3 in brain aging/AD as a therapeutic strategy.

Still referring to FIG. 13, studies of SB_NI_112 treatment in old wild-type mice (no pathology) and transgenic rtg4510 mice (tau pathology) were performed. We found that SB_NI_112 treatment: 1) reduced neuroinflammation, anxiety, and enhanced learning/memory in both models; 2) suppressed transcriptome signatures of neuroinflammation in old mice; 3) reduced misfolded protein accumulation (i.e., tau pathology); 4) had minimal off-target effects (using RNA-sequencing); 4) potentially preserved the ability to fight infections by specifically targeting the rogue inflammasome, while other key cytokines (e.g. IFN-γ) expression were unchanged; and 5) had positive systemic effects like reduced liver and heart inflammation, which could reflect improved cardiovascular and metabolic function (effects that would further reduce AD and dementia risk).

Still referring to FIG. 13, further studies may test the efficacy of SB_NI_112 for reducing neuroinflammation and protecting cognitive function in the settings of both AD-related pathology and aging. Based on our successful proof-of-concept studies in transgenic rTg4510 mice at 150 mg/kg SB_NI_112 (LP. injection), dose-finding studies using 2 translatable routes: 1) oral; and 2) subcutaneous, ranging from 1 -650 mg/kg (1, 5, 25, 125, 650 mg/kg, to identify the correct ranges, and then 2-fold changes), in 3 male and 3 female mice per group (7 groups oral dose, 7 groups subcutaneous, 14 groups total for both models) may be conducted. Such a study may be used to 1) Obtain dose-response curve, 2) identify dose with >50% improvement in cognition (using novel object recognition (NOR)) and 50% reduction in IL-1β expression in the hippocampus. Potential efficacy in age-related dementia using 20-month-old mice, and age vs pathology effects using transcriptome studies may also be done. Young and old wild-type mice may be treated with SB_NI_112, using the threshold doses identified in Aim 1 (50% and 90% threshold) and multiple measures of cognitive function may be evaluated. Brain region-specific transcriptomics (RNA-seq) may be performed to determine which pathways are modulated most. Such studies may result in >50% improvement in cognition (using NOR) and 50% threshold reduction in IL-1β and may reveal 36-different pro-inflammatory cytokine expression (evaluated using principal component) in the brain. 28-day GLP-toxicology safety studies in dogs may be performed. IND-enabling Chemistry, Manufacturing, and Control (CMC) and Safety-Toxicology studies for SB_NI_112 may be performed.

Still referring to FIG. 13, aging itself is the primary risk factor for AD, but the exact mechanisms by which brain aging contributes to the development of AD are unclear. Brain aging is associated with declines in cognitive function that can develop into mild cognitive impairment (MCI, a potential precursor to AD), and both brain aging and MCI are associated with the deposition of amyloid beta (Aβ and hyperphosphorylated tau (key pathological features of AD). These observations suggest that age-related processes play a central role in the development of AD, and among these, neuroinflammation is a particularly promising therapeutic target. Neuroinflammation is characterized by innate immune activation and the production of pro-inflammatory, CNS-toxic cytokines, especially by glial cells. It plays a central role in AD, as reflected by the fact that many AD risk genes have innate immune functions (e.g., CD33, TREM2). However, neuroinflammation also increases with normal brain aging, and may precede Aβ/tau pathology, increase further in response to pathology, and directly reduce cognitive function. As such, an important goal for pharmaceutical research is to identify interventions for reducing neuroinflammation in the context of aging and AD-related misfolded protein pathology.

Still referring to FIG. 13, activated abnormal activation of the inflammasome, specifically NLR family pyrin domain containing 3 (NLRP3), may be associated with a wide range of inflammatory, autoimmune, cardiometabolic, and neurodegenerative diseases. However, NLRP3 is also poorly understood. For example, designing antibodies to target NLRP3 effectively or target specific binding pockets of the protein through small molecules has been fairly challenging, making clinical translation difficult, and NLRP3 participates in multiple signaling pathways that may drive neuroinflammation. Among the many other possible targets besides NLRP3, one with key roles in brain aging and AD is nuclear factor kappa B (NF-κB). NF-κB is a central upstream transcription factor involved in many inflammatory processes and pivotal in “inflammaging”, as well as in NLRP3-associated inflammatory responses to both extracellular and intracellular stimuli, many of which are implicated in both aging and AD (FIG. 13). For example, multiple age- and AD-related signals (e.g., systemic inflammatory processes, immune dysregulation, etc.) elicit NF-κB signaling via Tumor Necrosis Factor (TNF) receptors and Toll-Like Receptors (TLRs), and NF-κB is also activated in response to intracellular stressors that increase with aging (e.g., reactive oxygen species and DNA damage). NLRP3 plays a major role in many of these events, and in responses to pathogen-associated molecular patterns (e.g., LPS, viral RNA), as well as mitochondrial reactive oxygen species/damaged DNA and senescence signals (all of which are hallmarks of aging). Moreover, NF-κB and NLRP3 may be involved in tau and AP pathology, and both interact with AD risk genes/proteins. Inhibiting NF-κB or NLRP3 pharmacologically or genetically may protect against neurodegeneration. However, therapeutics in this context are limited by: 1) potential off-target effects; and 2) failing to target both NF-κB and NLRP3. SB_NI_112 addresses all of these problems, and the cocktail potently inhibits neuroinflammation. SB_NI_112 reduces pro-inflammatory astrocytes/microglia even in prion-treated mice (a model of severe neurodegeneration), and is associated with cognitive improvements. Thus, targeting neuroinflammation with SB_NI_112 has promising therapeutic potential. It is also worth noting that gene targeting therapies delivery across the blood-brain barrier has been impossible, and they typically elicit a strong immunogenic response. Peptide nucleic acids (PNAs) can circumvent issues with immune activation and have several attractive molecular attributes for therapeutic development, but their neutral backbone makes delivery across cell membranes even more challenging.

Still referring to FIG. 13, PNA-based Nanoligomers may safely up-regulate (activate) or down-regulate (inhibit) any desired gene in any organism in a safe, effective, and targeted manner. In some embodiments, a nanoligomer may include: 1) a polynucleotide binding domain (which may be referred to as a DNA or RNA binding domain or DBD/RBD) that functions as the gene expression-modifying element; 2) cell uptake domain (CUD), to transport the peptide molecular assembly (Nanoligomer) across either specific tissues and organs, or non-specifically across different tissues and cell-types; 3) Nuclear localization sequence (NLS) peptide sequences for transport across the nucleus and DNA (or transcriptional) targeting; 4) peptides that can recruit more transcriptional activators, for gain-of-function; and 5) a highly engineered gold nanoparticle (made from FDA generally regarded as safe or GRAS materials) that aids in purification and enhances the transport of the molecule into target cells.

Still referring to FIG. 13, a nucleic acid-binding domain (RBD/DBD) may include a peptide nucleic acid (PNA), a synthetic DNA analog in which the phosphodiester bond is replaced with 2-N-aminoethylglycine units. PNAs demonstrate strong hybridization and specificity to their targets compared to naturally occurring RNA or DNA, and they exhibit no known enzymatic cleavage, leading to increased stability in human blood serum and mammalian cellular extracts. These advantages make PNAs a promising choice for expression-modifying therapeutics, but they are neutral molecules and therefore their past implementation has suffered from transport challenges. Nanoligomers improve both purification and transport, resulting in a self-contained, “transfection-free” nucleic acid therapy that can be designed to bind to mRNA or double-stranded DNA. Nanoligomers designed to alter gene expression bind to the respective promoter regions and include domains to bind transcriptional activators or repressors, thereby activating or repressing transcription, as desired. Nanoligomers designed to inhibit the translation of target mRNAs simply binds to the mRNA which prevents access to ribosomes.

Still referring to FIG. 13, to develop brain-penetrant SB_NI_112, an artificial intelligence (AI) platform may be used to rank potential PNA sequences aimed at regulating genes of interest and identify the best molecule/sequence with minimal off-targeting. Candidate Nanoligomers may be tested in 3D human iPSC-derived brain neurospheroids, specifically for their ability to downregulate neuroinflammation-associated cytokines and inflammasomes. SB_NI_112 is more stable than other nucleic acid therapies, has proven delivery in the brain, requires no special formulation, and can be administered directly in animals.

Still referring to FIG. 13, brain penetrant nanoligomers targeting NF-KB and NLRP3 may inhibit up and downstream causes of neuroinflammation in aging/AD.

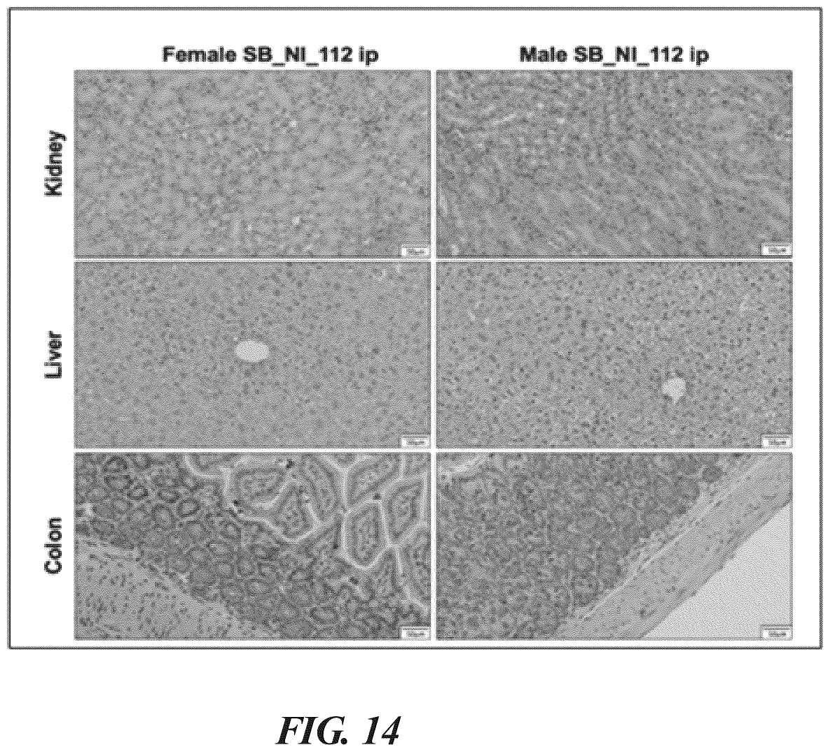

Now referring to FIG. 14 and also referring back to FIG. 10, brain-penetrant SB_NI_112 can be delivered easily, shows good bioavailability in different brain regions, with no immunogenic response or accumulation in first-pass organs. We have conducted detailed pharmacokinetic (PK), bioavailability, safety-toxicology studies in small animals (mice) to show that all three routes of administration (injectable, oral, and inhalable) can be used for delivery of SB_NI_112 across different brain regions. Although the Kd value of SB_NI_112 is low (2-15 nM), low-to-medium dosing (˜150 mg/kg) shows high bioavailability across different brain regions (analyzed using inductively coupled plasma mass spectrometry ICP-MS analysis), with drug profile closely matching the protein expression (FIG. 10). The large measured half-life (˜24 days), renal clearance of the drug in <24 hours with missense/non-host targeting sequences, and multiple translatable routes represent good pharmacological properties. Additionally, detailed analysis of first pass and clearance organs (kidney, liver, colon) show no accumulation, and H&E-stained images (FIGS. 14), and 36-cytokine ELISA show no immunogenic response.

Still referring to FIG. 14, H&E stained first pass and clearance organs with no accumulation or immunogenic response using 500 mg/kg dosing of NF-κBNLRP3 downregulating SB_NI_112 treatment is shown.

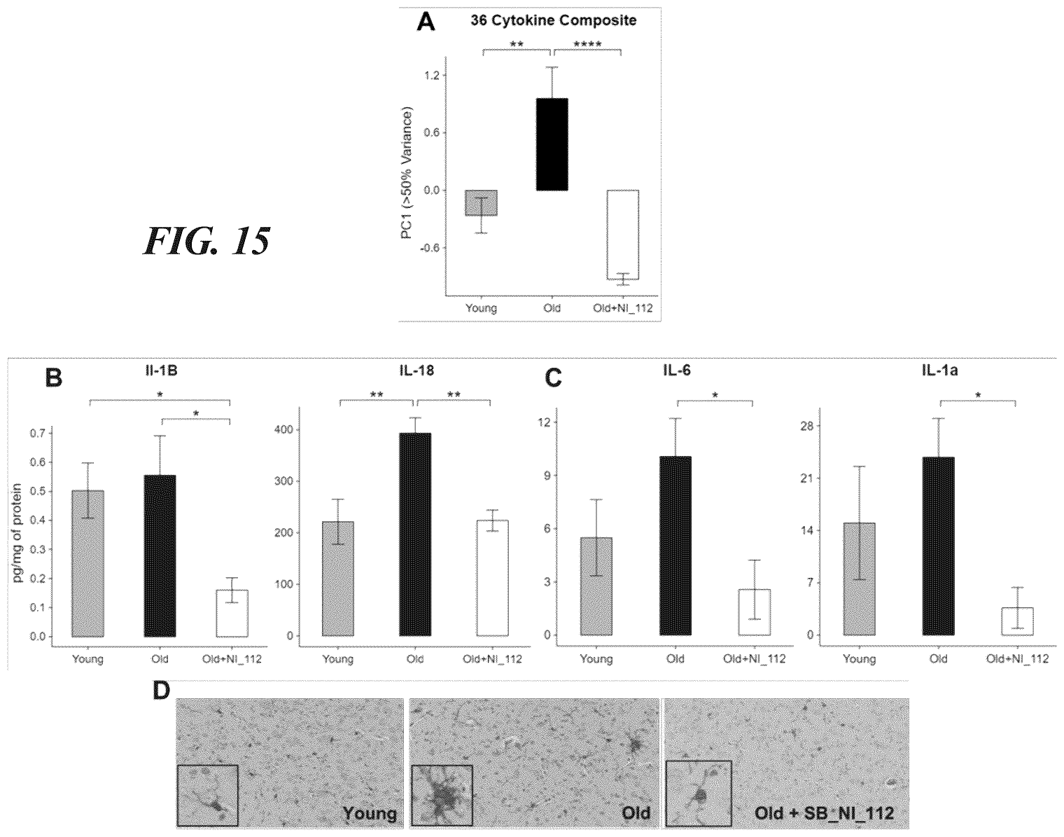

Now referring to FIG. 15, reduced neuroinflammation and microglial activation in old mice with 4-week treatment was found. We assessed the impact of NF-kB/NLRP3 downregulating SB_NI_112 in 20-month-old mice, with 4 weeks of treatment. As seen using multiplexed ELISA of the hippocampus in FIG. 15. Using principal component analysis of all 36 cytokines to assess neuroinflammation and neurodegeneration, a clear reduction in neuroinflammation can be seen across the board in old mice, within just 4 weeks (FIG. 15A). Especially, high target engagement (shown using IL-1β and IL-18 expression, FIG. 15B), and a clear reduction in key cytokines like IL-6, IL-1α (FIG. 15C) in the brain shows clear evidence of reduced neuroinflammation and biomarkers associated with “inflammaging”. Further evidence of reduced microglial activation was seen in different brain regions in a detailed histological examination of brain regions using Iba-1 (for activated microglia, FIG. 15D), and GFAP staining (for activated astrocytes). A clear reduction in activated microglia and astrocytes points to the potential reduction/blocking of inflammatory pathways implicated in the neuropathology seen in a number of neurodegenerative diseases.

Still referring to FIG. 15, data showing that NF-KB/NLRP3 downregulating SB_NI_112 treatment (A) reduces multiple neuroinflammation-associated cytokines and (B) shows high target engagement as seen using IL-1B and IL-18 expression are provided. Also provided is (C) data on other key cytokine changes, and representative immunohistochemistry staining for Iba1 (activated microglia). *P<0.05, N=6-8/group, means +/−SD, 4 week IP treatment.

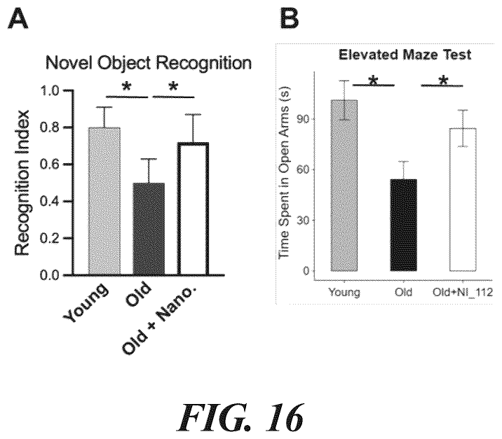

Now referring to FIG. 16, improved cognition, reduced Anxiety, and marginal improvement in grip strength is shown. We also conducted a battery of behavioral and cognitive tests in young, old, and brain-penetrant SB_NI_112-treated mice. 1) Novel Object Recognition (NOR, learning/memory): Mice were habituated to 2 identical objects; after 24 h, one was replaced with a new object. Recognition index was quantified as time exploring new vs. old. 2) Elevated Plus Maze (anxiety/behavior): Mice were placed in an apparatus consisting of 2 open and 2 closed arms. Anxiety-like behavior will be measured as time/distance achieved in closed vs. open arms. 3) Basic motor function tasks (to evaluate potential neuromuscular improvements): Grip strength and rotarod endurance/latency to fall were evaluated using standard procedures and equipment (Maze Engineers). The treatment improved learning and memory, evaluated using NOR, in just 2 weeks (and then further in 4 weeks, FIG. 16A). While there was a clear reduction in anxiety (FIG. 16B) and marginal improvement in grip strength (p=0.09, associated with cognition), other motor functions such as frailty index, rotarod endurance, etc. were unchanged, pointing to clear improvement in cognition and brain function specifically, with similar muscular function.

Still referring to FIG. 16, data showing that SB_NI_112 treatment (A) improves learning/memory and (B) reduces anxiety-like behavior in old mice is provided. *P<0.05, N=6-8/group, mean +/−SB, 4 week IP treatment.

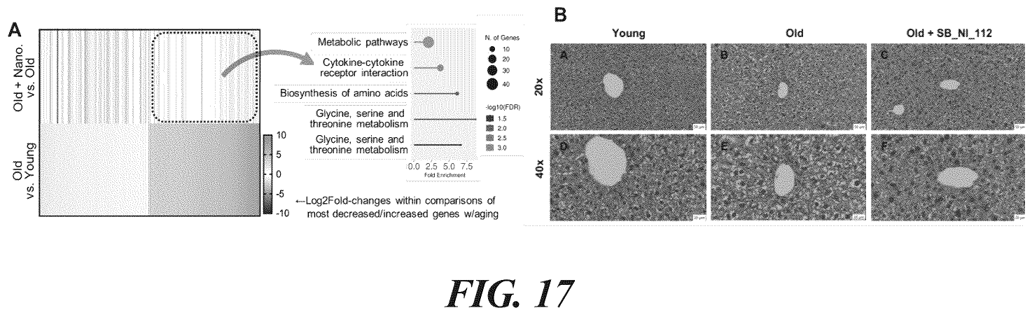

Now referring to FIG. 17, data showing systemic improvement in chronic inflammation (“inflammaging”), potential impact on cardiovascular and metabolic function in old mice is provided. Inflammaging contributes to declines in multiple physiological functions, and cognitive function is impaired by both neuroinflammation and peripheral inflammation. These events are also linked with many other adverse aging processes, and as a result, “inflammaging” is among the most central hallmarks of aging. For example, DNA damage, protein misfolding, senescence and mitochondrial damage are reported to both cause and increase in response to inflammaging. These processes also engage NF-κB and NLRP3, which drive immune responses to bacterial toxins, DNA/mitochondrial damage and other intracellular signals. In the brain, these events are particularly relevant, as neurons are less able to diffuse intracellular damage and glial cells are increasingly reactive at baseline. Together, these events cause declines multiple domains of cognitive function (e.g., memory, processing speed, executive function) and an increase in anxiety-related behaviors. They also eventually impair activities of daily living and increase the risk for MCI/AD. Given these observations, to evaluate potential mechanisms of action for SB_NI_112 in our studies described above, we performed a pilot RNA-seq study on mouse hippocampus (FIG. 17A). We found clear evidence of reduced cytokine-related signaling with SB_NI_112 treatment, which prompted further investigation of potential systemic improvements in cardiovascular and metabolic function, given the key role of inflammation in inducing age-related pathology in these tissues. Using H&E-stained heart and liver of old treated, untreated, and young mice, we observed several key data points that show reduction in systemic inflammation. First, we observed more polyploid hepatocytes with large nuclei in the aged mice, but were significantly reduced in those that were treated, and not present in young mice livers (FIG. 17B). This observation together with reduced inflammaging typically indicates lower terminal differentiation and senescence, which can improve outcomes for age-related cardiometabolic diseases too. Further, we also observed a clear polymorphonuclear leukocyte aggregation around the hepatic portal vein of the untreated old mice, but mostly absent in young mice and old treated mice, which is additional evidence for reversal of age-related inflammation in hepatic tissue (FIG. 17B). These findings clearly reveal that SB_NI_112 has excellent systemic benefits in reducing age-related inflammation and can contribute to significant improvement in cardiometabolic function, thereby further contributing to healthy aging.

Still referring to FIG. 17, NF-KB/NLRP3 nanoligomer treatment reduces systemic inflammation and causes clear change in transcriptome signatures in old mice. (A) Heatmap of top age-related RNA-seq changes; genes/KEGG processes reversed by treatment include cytokine signaling. (B) Representative immunohistochemistry staining for H&E stained liver. Old mice livers showed more polyploid hepatocytes with large nuclei which reduces with treatment, indicating terminal differentiation and senescence is reduced in liver with SB_NI_112 treatment. Aged mice also show polymorphonuclear leukocyte aggregation around the vein (no treatment) liver, suggesting age-related inflammation, rescued with treatment. 4 week IP treatment.

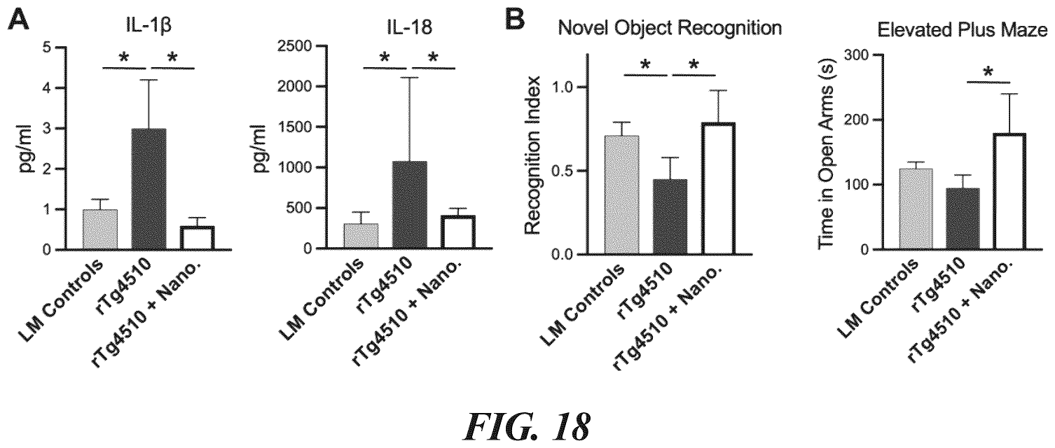

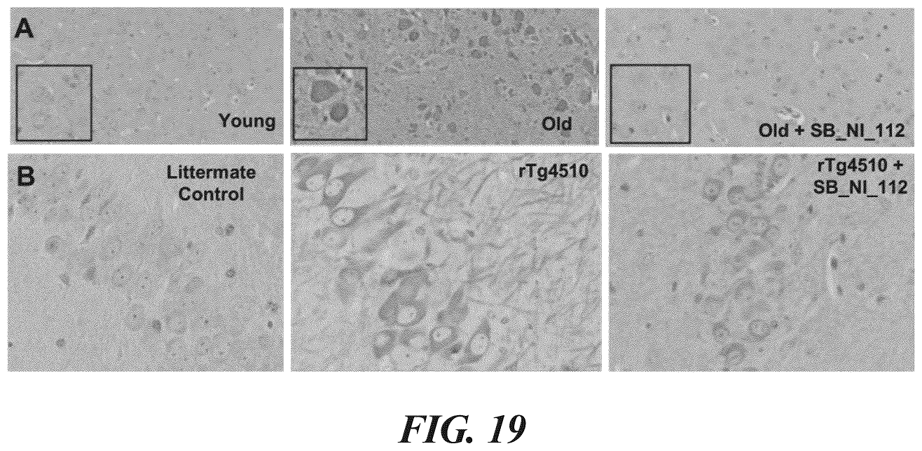

Now referring to FIGS. 18 and 19, improved cognition in AD transgenic model for tauopathy is shown: While there is no perfect mouse model for AD, for proof of concept, we performed a pilot study of NF-κB/NLRP3-downregulating SB_NI_112 Nanoligomer treatment in rTg4510 mice, a reproducible model of tauopathy based on the expression of mutant (P301L) human tau. These mice develop neurofibrillary tangles, neuroinflammation, and cognitive dysfunction starting at 2-4 months of age. However, we found that 4 weeks of SB_NI_112 treatment starting at 3 months of age reduced neuroinflammation (similar effects on 36 cytokines in old mice), improved measures of cognitive function, and reduced anxiety-like behavior in these animals (FIG. 18). Additional histological analysis of brain regions showed clear evidence of increased tau pathology in old mice and rTG4510 mice, compared to treatment (FIG. 19). Therefore, this pilot study showed further evidence of potential for reduction in misfolded proteins with just 4 weeks of treatment in old mice and needs further examination through a combination of tau and Aβ models proposed in this application.

Now referring to FIG. 18, NF-κB/NLRP3 nanoligomer treatment SB_NI_112 (A) reduces neuroinflammation and (B) improves learning/memory and reduces anxiety-like behavior in rTg4510 (tauopathy) mice. *P<0.05, N=4-6/group, mean +/−SD, 4 week IP treatment.

Now referring to FIG. 19, representative immunohistochemistry staining for T-217 stain for phosphorylated tau in (A) old mice study; and (B) rTG4510 mice. Box in old mice brains shows increased phospo tau, whereas transgenic and old mice show significant reduction in misfolded tau protein with just 4 weeks of IP treatment.

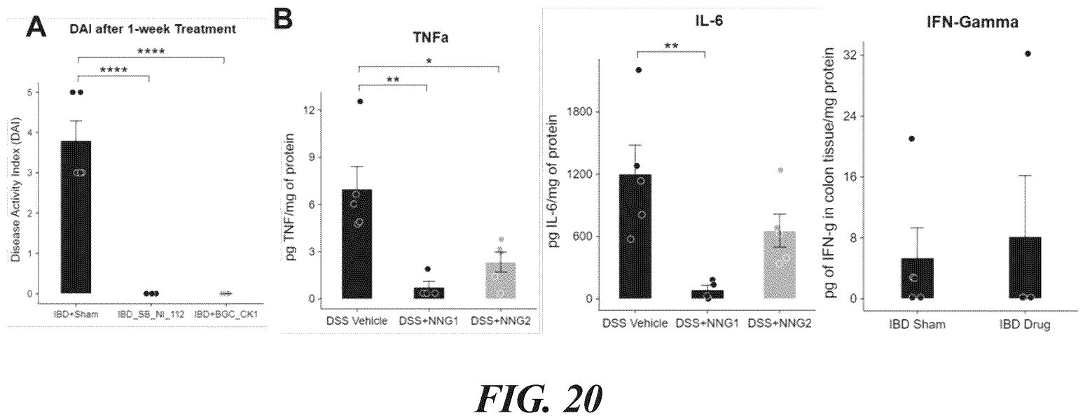

Now referring to FIG. 20, data on treatment of autoimmune diseases, while preserving the ability to fight infections is provided. In a recent pre-clinical study for assessment of autoimmune disease (ulcerative colitis, which increases the risk of dementia in humans) in mice, we used chemically induced (using DSS-induced colitis) and genetically engineered mice (TNF^{ΔARE}) with their guts populated with microbiome using fecal matter from inflammatory bowel disease (IBD) patients. Following just 3 treatments of SB_NI_112 in less than 1 week, the mice with serious colitis (watery stool and blood) showed complete remission (disease activity index DAI went from 4 to zero, FIG. 20A), and biochemical analysis of colon tissue revealed >90% reduction in key biomarkers such as TNF, IL-6 (FIG. 20B). While biochemical and histology data showed a strong reduction in inflammation and shift in the microbiome, key cytokines such as IFN-γ showed that the mice potentially retained their ability to fight infections (FIG. 20B), due to specificity of targeting using Nanoligomers.

Still referring to FIG. 20, NF-κB/NLRP3 nanoligomer treatment SB_NI_112 (A) reduces autoimmune IBD DAI scores. (B) Specific cytokine expression in colon (TNF, IL-6) shows >90% reduction in inflammation, while preserving the ability to fight infections (BGC_CK1 is personalized microbiome targeting Nanoligomer cocktail treatment). *P<0.05, **P<0.01, ****P<0.0001, N=4-6/group, mean +/−SD, 1 week IP treatment.

Still referring to FIG. 20, the brain penetrant SB_NI_112 offers the following potential advantages over other drugs: 1) Orthogonal mechanism of action compared to standard of care treatments and can be used in combination to improve cognition and memory. Standard-of-care drugs are cholinesterase inhibitors and N-methyl-D-aspartate (NMDA) receptor antagonists, which are routinely used in combination with other medications to treat other symptoms or conditions, such as depression, sleep disturbances, hallucinations, parkinsonism, or agitation. Our lead molecule targets upstream inflammasome pathways and have shown strong improvement in cognition, memory, reduced anxiety, and misfolded protein concentration in the brain such as phosphorylated tau, and amyloid beta. 2) High safety profile and potentially preserves the ability to fight infections. Almost all neuroinflammation treatments such as steroids and immune suppressants require prior testing for pre-existing conditions and infections. However, as a result of massive COVID outbreaks and coupled outbreaks and spread of RSV and Flu, neurologists have started to pay more attention to this aspect given the chronic and long-term dosing required to assess cognition improvement. 3) Strong systemic benefits for cardiometabolic function. This is especially important for the patients and clinicians since many patients suffer from pre-existing cardiometabolic issues/pathology with age, and this positive systemic effect was highly valued during customer discovery.

Still referring to FIG. 20, AD and MCI is a complex disease, and the poor results of clinical testing of several high-profile therapeutics and immunosuppressants highlights the challenges of treating the disease. Nanoligomers may be deployed as a stand-alone, first-line therapy for some patients. However, the complexity of disease and disease advancement could mean standard-of-care treatments may be used as a co-treatment. Nanoligomers may also be used as an adjuvant to cholinesterase inhibitors and NMDA antagonists. Nanoligomers may be used as a prophylactic.

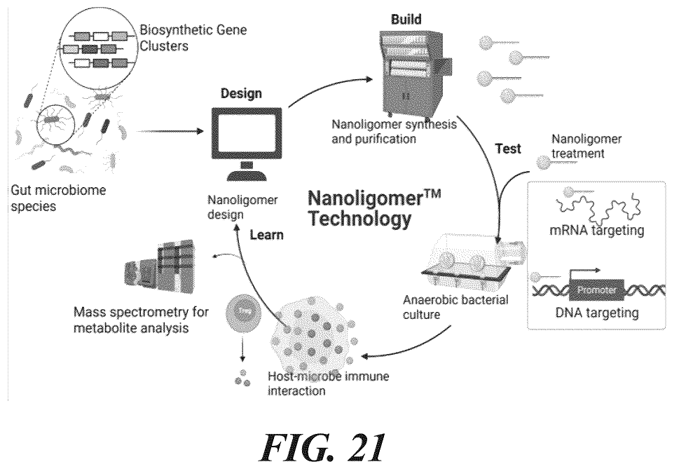

Now referring to FIG. 21, inflammatory bowel disease (IBD) is a complex illness with inadequate treatment options. Approximately 1.6 million Americans suffer from Crohn's disease or Ulcerative colitis, collectively described as inflammatory bowel disease (IBD). In the US alone, costs associated with IBD are estimated to be>$31 B each year, in part due to the persistence of illness in patients who do not achieve complete remission with current treatments. The etiology of IBD is not entirely understood, but broad evidence indicates the hallmark chronic inflammation stems from abnormal immune responses and alterations in gut microbiota, triggered by diet or other environmental causes in individuals who are genetically susceptible. The standard of care for IBD varies depending on disease severity and other factors, but it typically includes immunosuppressants that increase the long-term risk of cancer and achieve complete remission in less than half of patients.

Still referring to FIG. 21, given that immunosuppressants are not adequate for all patients, researchers have turned to the altered gut microbiome as a target for novel therapies. Delivery of selected microbes (bugs as drugs), fecal matter transplant (FMT), and genetically engineered probiotics have been proposed as potential therapies targeting the microbiome. Each approach is designed to shift the composition and relative abundance of bacteria to better match individuals without IBD, with the hope that such a shift will restore an anti-inflammatory environment and eliminate the bacterial metabolites that contribute to the altered immune response. Of the more than 300 ClinicalTrials.gov-registered studies of FMT for various indications, only one has successfully completed Phase 3 trials (for intractable Clostridium difficile infections). Eight Phase 3 trials are testing FMT in IBD, but none are being conducted in the US. Of ˜30 Phase 2 trials, 15 have been completed, terminated, or withdrawn, and none of the trials completed in the US have advanced to Phase 3. Based on multiple reports of adverse events, FDA has also issued a warning about the risk of disease transmission and morbidity with FMT, which may ultimately limit further development.

Still referring to FIG. 21, remission rates for IBD treatment using generic anti-inflammatory therapies is low (<50%). Nanoligomers may be used to target specific upstream mediators of inflammation for therapeutic efficacy. Our studies have shown that specifically: 1) Nanoligomers reduce key cytokine mediators of IBD, 2) Nanoligomers are delivered to the gut using oral or intraperitoneal (IP) administration, requiring no special formulation. Nanoligomers are differentiated from the current standard of care (SOC) treatments in at least four ways: 1) Reduced inflammation as evidenced by decrease of key cytokines targeted by blockbuster drugs. 2) Not affecting the ability to fight infections as evidenced by no changes in the interferon pathway. 3) Expression of growth factors responsible for healing are kept at levels similar to healthy animals. 3) Oral administration vs. current SOC which is only available as injectable or infusion.

Still referring to FIG. 21, a nanoligomer platform for targeting Biosynthetic Gene Clusters (BGCs) in gut microbes is shown. Nanoligomers may combine nucleic acid-binding PNAs with an engineered nanoparticle to achieve entry into cells where they may bind DNA at the promoter region of a target gene to enhance or inhibit transcription or may bind a target mRNA to prevent translation.

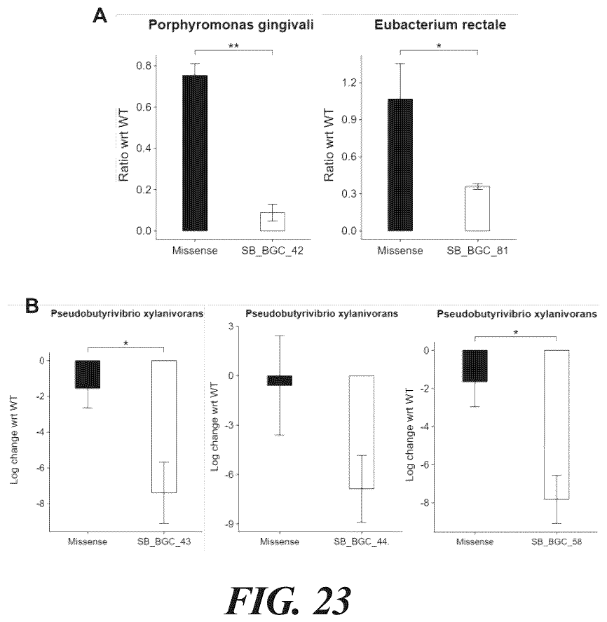

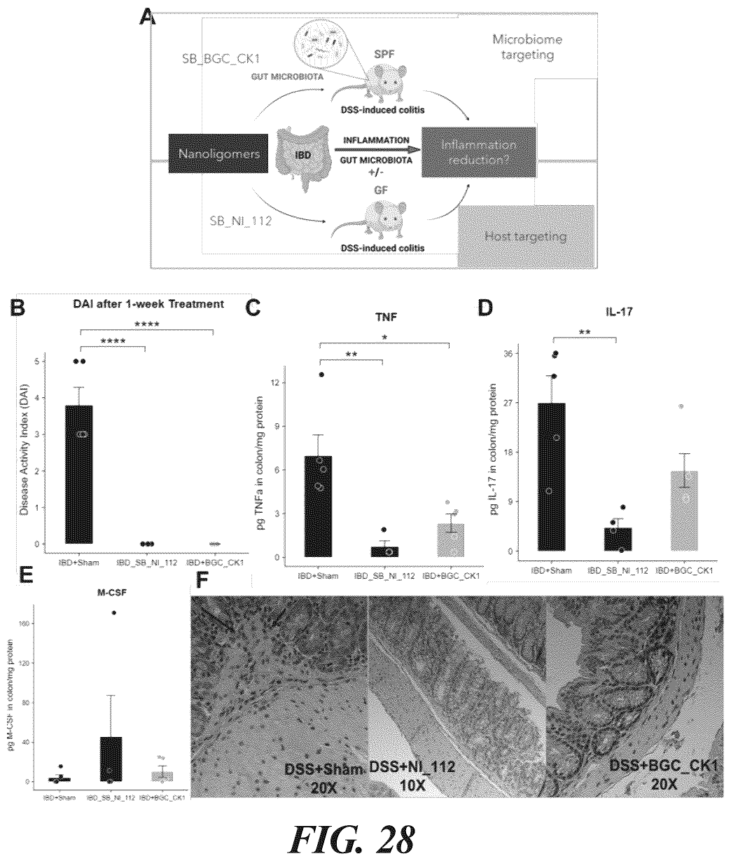

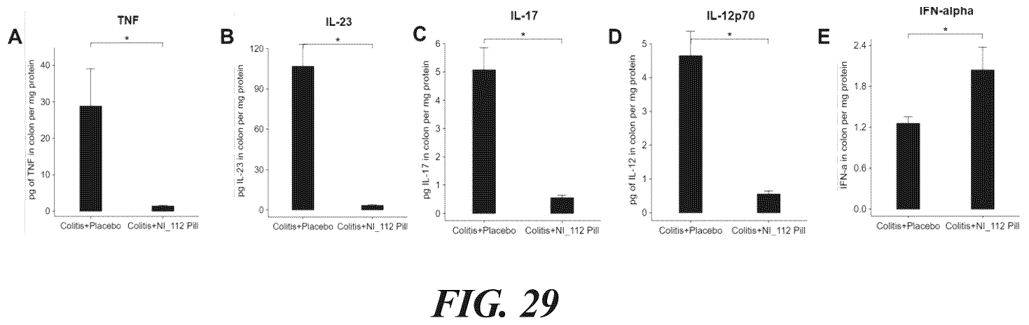

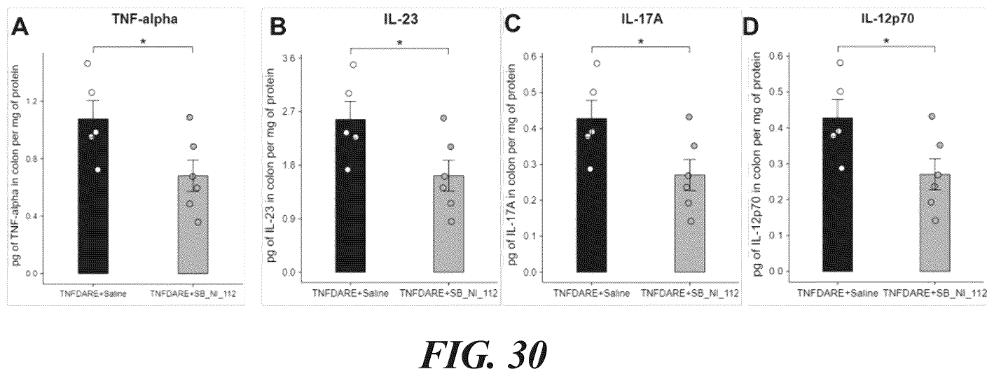

Now referring to FIGS. 21-31, we evaluated and compared the results and mechanism of Nanoligomers with other marketed and IBD drugs under development (clinical evaluation). First, since the microbiome-targeting Nanoligomer therapeutic targets either human microbiome (BGC_CK1) or human inflammatory pathway (NI_112), and therefore is more upstream/causal compared with other therapeutics in the market/under development. Consequently, we observed that the lead molecules affected/reduced key biomarkers for the top-selling IBD drugs without compromising the ability to fight infections as evidenced by no suppression of the interferon pathway (FIGS. 23-30). For example, besides TNF, other damaging cytokines such as IL-17, IL-23, IL-12 etc. were also reduced significantly (80 and 63%) by the lead molecules (NI_112 and CK1) in colon tissue (FIGS. 28C, 29, 30). Further, top-selling IBD drugs like Stelara target IL-12 and IL-23, which are also downregulated by 90% and 85% by the Nanoligomer molecules (FIGS. 28C, 28D, 29, 30). A more systematic evaluation revealed the Nanoligomer lead molecules combined the mechanism of action of all top-selling drugs like Humira®, Remicade®, Stelara®, Skyrizi®, Tryemfya®, Coseyntix®, Taltz® as observed in both chronic DSS-colitis (FIGS. 28-29), and genetic TNF^{ΔARE/+} mice (FIG. 30) models. Further, 14-day Safety-Tox and tolerability studies in large animals (beagle dogs) showed that: 1) Nanoligomers are safe and have excellent bioavailability, 2) identify MTD for three translatable routes (IV, SQ, and PO), and 3) provide 14-day safety-tox data and monitoring of organ health using detailed CBC/Chem (FIG. 31).

Still referring to FIGS. 21-31, the evaluation of mouse immunological endpoints and in vitro PBMC evaluation revealed that both Nanoligomer lead molecules could potentially preserve the ability to fight infections. This can also be seen in the unchanged expression of IFN-α,γ in the mouse cytokine expression (FIG. 29E). This could again be a key product attribute desirable by clinicians and patients suffering from moderate to severe UC and Crohn's. Almost all current treatments available for IBD require prior testing for pre-existing conditions and infections, especially upper airway respiratory infections. However, as a result of massive COVID outbreaks and coupled outbreaks and spread of RSV and Flu, many clinicians have started to pay more attention to this key aspect.

Still referring to FIGS. 21-31, especially for moderate to severe UC and Crohn's patients, gastroenterologists and patients prefer a drug that promotes ulcer healing. On close evaluation of both histology, as well as cytokine expression of key growth factors, both lead Nanoligomers showed either an unchanged or slightly increased expression of growth factors in the colon (like M-CSF, FIG. 28E, 28F). This is especially important for the patients and clinicians since the alternative for many patients (assessed during customer discovery) was much more expensive emergency room visits or surgery, with long and painful recovery periods.

Still referring to FIGS. 21-31, IBD is a complex condition, and the mixed success of immunosuppressants highlights the challenges of treating the disease. Nanoligomers may be deployed as a stand-alone, first-line therapy for some patients. Nanoligomers may be used as an adjunct to immunosuppressive therapies (such as to aid the >50% of patients who do not completely respond to immunosuppressants). Nanoligomers may be used as a prophylactic to prevent a recurrence.

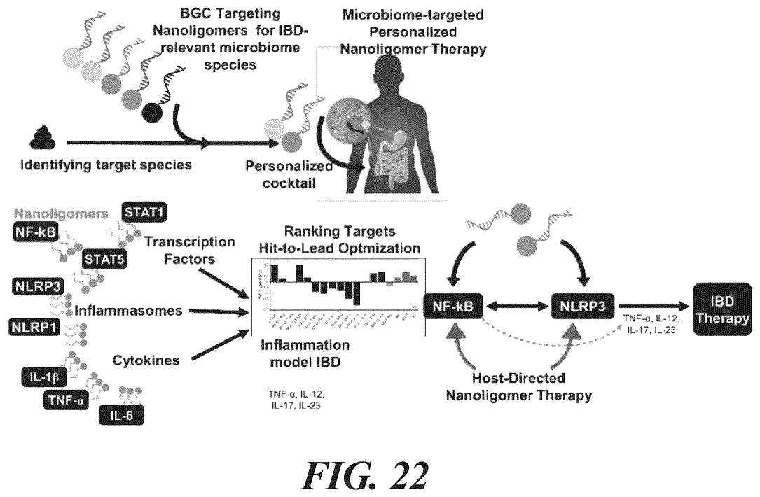

Now referring to FIG. 22, we screened 70 BGCs and corresponding 42 microbial species from either metagenomic sequencing of human poop samples, and/or annotated BGCs and their metabolites from databases such as antiSMASH 6.0 and 5.0, BiG-FAM, DoBISCUIT, and MIBiG 2.0 (FIG. 22). Some examples of human microbiome species included Bacteriodes fragilis, Eubacterium rectale, Faecalibacterium prausnitzii, Blautia coccoides, Blautia hansenii, Roseburia hominis, Clostridium sporogenes, Ruminococcus (Blautia) obeum, Akkermanisa muciniphila, Ruminococcus gnavus, Alistipes shahii. Examples of BGCs include C4Q21_RS06440, WP_014080837.1,Clospo_02864, ZP_01962381,WP_000945878.1 etc. We prioritized list of metabolites and strains that are hypothesized/known to produce immunomodulatory effects, and prioritized BGC targeting these metabolites, to assess their impact and prioritize them.

Still referring to FIG. 22, selection criteria used to identify personalized microbiome targeting cocktail SB_BCG_CK and host directed therapy SB_NI_112 are shown.

Now referring to FIG. 23, the designed Nanoligomers were able to selectively target the BGCs with high efficacy, and were validated for modulation of gene expression of corresponding BGCs using qPCR. Overall, ˜70-measured Nanoligomers targeting BGC met a minimum threshold of 2-4 fold decrease (of target genes) in gene expression, with respect to both wild-type (wrt WT) and missense (non-targeting) Nanoligomers.

Still referring to FIG. 23, these Nanoligomers were used for further analysis of immune modulation with donor-derived human Peripheral Blood Mononuclear Cells (PBMCs), of a gut-immune model. We used a gut-immune model where microbiome cultures (in their growth phase) were treated with Nanoligomers targeting the BGCs, and then either the: a) cell supernatants; or, b) cell lysates (all filtered sterilized with 0.2 μm filter), were added to donor-derived human PBMCs, to monitor their effect on host immune system. The cell lysates could stimulate the PBMCs by themselves, whereas for cell supernatants, we used PMA (25 ng/ml) and ionomycin (1 μg/ml) for 6 hours. All cytokines measured in the PBMC supernatants were analyzed using multiplexed 65-panel ThermoFisher ProcartaPlex Human Immune monitoring panel, and all treatments were normalized with their respective untreated controls.

Still referring to FIG. 23, data on nanoligomers selectively targeting BGCs and immunomodulatory metabolites in target species is shown. (A) BGC-targeting nanoligomers SB_BGC_42 and 81 show >70% downregulation of target gene, compared to WT and missense (non-targeting_ Nanoligomers, in BAA-308 and ATCC 33656, (B) Nanoligomers SB_BGC_43, 44, and 58 show 6-8 log decrease (>98.4 to 99.6% decrease) compared to missense Nanoligomer, in BAA-455. While nanoligomers show significant reduction in gene expression, the measured qPCR values also depend on mRNA stability.

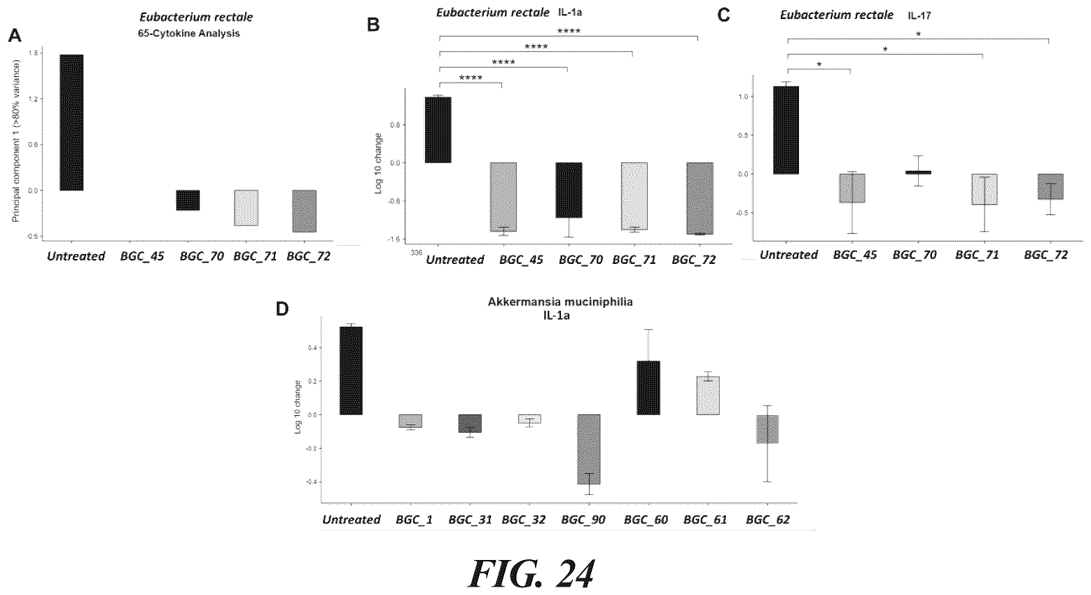

Now referring to FIG. 24, immune expression profile of treated cell lysates is compared. Using Eubacterium rectale (ATCC 33656) and Akkermansia muciniphila (BAA-835) as two specific examples, we show selective BGC (and immuno-modulatory metabolite encoded by those BGCs) in FIG. 24. Using a 65-cytokine composite shown in FIG. 24A for ATCC 33656, the donor-derived PBMCs showed a strong anti-inflammatory effect on down-regulating BGCs 45, 70, 71, and 72. Using 2 specific cytokines as examples of immune modulation, IL-1α) (key proinflammatory cytokine mediator for IBD78, FIG. 24B) and IL-17 (heavily implicated in IBD and target of many therapeutics, FIG. 24C), we demonstrate (on a Log-10 scale) that all target BGCs (45, 70, 71, and 72) reduced inflammation by ˜20-fold. However, these all target the same strain, and hence only the top 2 molecules (SB_BGC_71, SB_BGC_72) for E. rectale were advanced to create the therapeutic cocktail. Further, many different Nanoligomer targeting BGCs in Akkermansia muciniphila (BGCs 31, 90) also met success criteria, but due to the abundance of options, only the top Nanoligomer target (SB_BGC_90) was advanced/included in the microbiome targeting cocktail for in vivo testing in IBD mouse models (chemically-induced DSS colitis, and genetic TNF^{ΔARE}).

Still referring to FIG. 24, data showing that nanoligomers targeting key immunomodulatory metabolites cause change in host immune response is shown. (A) 65-cytokine composite analysis in Eubacterium rectale using principal component 1 (84.1% variance) shows reduction in multiple inflammatory cytokines, with varying degree of efficacy. Specific cytokines on Log-10 scale for (B) IL-1* and (C) IL-17 shows high reduction in inflammation (>20-50 fold), using gut-immune in vitro model. (D) Log-10 reduction in IL-1α Akkermansla muciniphila showing that targeting only selective metabolites (those targeted by BGCs 1, 31, 32 and 90) show reduced inflammation, whereas others can increase inflammation. All data shown is statistically significance and significance marks are removed from (A) and (D) for clarity.

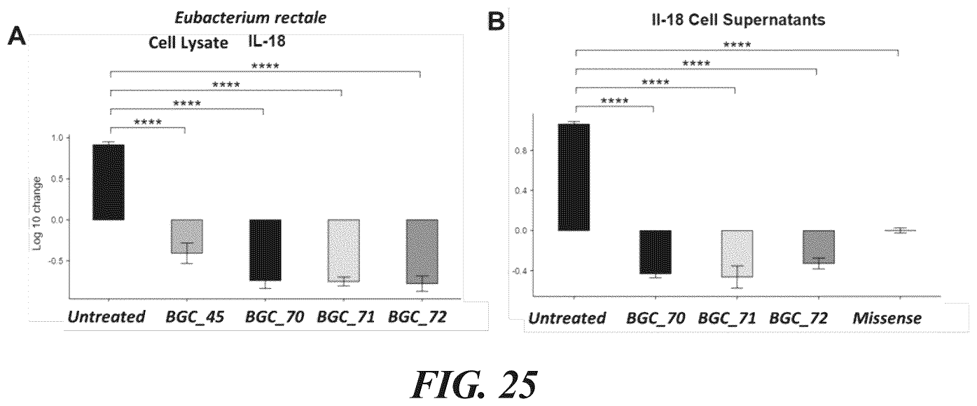

Now referring to FIG. 25, immune expression profile of treated cell supernatants is compared. First, we showed the effect of cell-lysates (above), and how top Nanoligomer targeting specific BGCs were identified. In parallel, we also treated donor-derived PBMCs using cell supernatants of the untreated and top Nanoligomer treated cultures. While the 65-cytokine immune profiles are different, key outcomes and results were consistent. One key difference was the IL-18 expression was the key pro-inflammatory output in cell supernatants (not IL-1α, IL-1β, IL-17 etc.), likely due to early profiling (6-hour supernatant treatment of PBMCs instead of 24-hour cell-lysate treatment protocol established in the gut-immune models used) of developing immune-modulation. Comparing the two models used in parallel (in Log 10-scale) in FIG. 25, top Nanoligomer molecules and targets were similar, although the ranking of E. rectale (ATCC 33656) targets changed slightly. However, multiple top BGC targeting Nanoligomers (SB_BGC_70, 71, and 72) again meet the success criterion of more than 2-3 fold reduction of host inflammation. This was also verified for key molecules selected and advanced for in vivo evaluation in IBD mouse models.

Still referring to FIG. 25, a comparison of gut-immune response in vitro model for cell lysate and cell supernatant comparison using IL-18 expression by donor-derived PBMCs is shown. (A) cell lysates show strong (>4-fold) reduction in IL-18 expression using top BGC targeting Nanoligomers. (B) Cell supernatants from the ATCC 33656 (E. rectale) treated with top nanoligomers also shown significant (>3-fold) reduction of host inflammation.

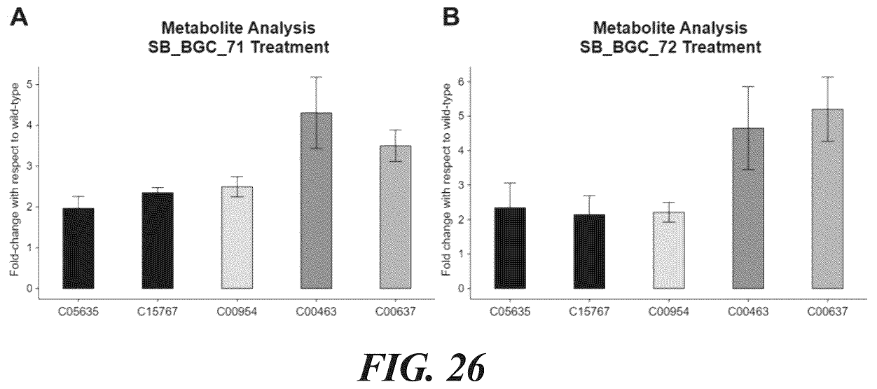

Now referring to FIG. 26, data on nanoligomers altering metabolite profiles of genetically intractable gut anaerobes is provided. To assess whether the changes in metabolite profile can modulate immune responses, we collected lysates and supernatants from the Nanoligomer-treated microbiome and assessed the effect of BGC targeting on metabolites using LC-MS profiling. While more than 2000 targeted and untargeted metabolites were identified, and a significant fraction of them (>25%) were differentially expressed, to provide a glimpse into key immunomodulatory metabolites, we focused on the short-chain fatty acid (SCFA) derivatives, which are important for colonocytes growth and metabolism and promoting a healthy gut microbiome, as well as some selected metabolites in the indole tryptophan pathway. For clarity, we are also showing the top 2 BGC treatments for Eubacterium rectale, as identified using in vitro gut-immune model. The following metabolites are shown here for these Nanoligomer treatments (compound IDs: [C05635]: 5-Hydroxyindoleacetate; [C15767]: gamma-Glutamyl-gamma- aminobutyrate; [C00954]: Indole-3-acetate; [C00463]: Indole; and [C00637]: Indole-3-acetaldehyde. As seen in FIG. 26A for BGC 71 treatment, all key SCFA derivatives were upregulated by more than 2-fold (all p-value<0.001). SCFAs participate in the maintenance of intestinal mucosa integrity, improve glucose and lipid metabolism, control energy expenditure, and regulate the immune system and inflammatory responses. Given their strong anti-inflammatory role, modulation of gut epithelial cells and preventing/reversing gut-dysbiosis linked to IBD and colorectal cancer, and promoting a healthy gut microbiome, the more than 2-fold upregulation seen with top BGC targeting Nanoligomers meet success criteria. Additionally, using the indole and tryptophan metabolite analysis, we clearly see significant upregulation of these key anti-inflammatory and barrier integrity strengthening metabolites by more than 2-fold (FIG. 26).

Still referring to FIG. 26, data on nanoligomer treatments altering the metabolite profile of Eubacterium rectale (and other gut anaerobes) is shown. More than 2000 metabolites were analyzed in cell lysates of wild type strains (ATCC 33656 E. rectale here), and corresponding Nanoligomer treated samples. The data shown here presents a small cross-section of data focused on short chain fatty acids (SCFA) derivatives and Indole-Tryptophan pathway. (A) Differentally expressed metabolites indicated by their corresponding compond iIDs: C05635: 5-hydroxyindoleacetate; C15767: gamma-Glutamyl-gamma-aminobutyrate; C00954: Indole-3-acetate; C00463: Indole; and C00637: Indole-3-acetaldehyde. More than 2-fold enhancement in SCFA derivatives and these key metabolites explains anti-inflammatory and gut-protective immune response observed in gut-immune in vitro model, and in vivo IBD studies. (B) Similar metabolites shown for SB_BGC_72 treatment. All p-values <0.001.

Now referring to FIGS. 14, 22 and 27, data on Host-targeting Nanoligomer SB_NI_112 is provided. From literature we identified ˜100 host immune gene targets and designed Nanoligomers (3/target) for each one (FIG. 22). b) Synthesized Nanoligomers, and c) screened them in multiple parallel in vitro inflammation models, including in PBMCs, human primary astrocytes, as well as human iPSC derived brain organoids, and measured 65-plex human cytokines. We identified top candidates that suppressed pro-inflammatory cytokines, and found that synergistic combination of NFkB and NLRP3 (SB_NI_112) in 1:1 ratio was most effective. d) Tested the brain-penetrant version of SB_NI_112 in vivo models of neuro-inflammatory diseases (including Multiple Sclerosis, and Alzheimer's), and validated target engagement as well as disease modification capabilities. Based on these results, a peripherally-restricted version of SB_NI_112 was used for auto-immune disease, in this case, Ulcerative colitis. We also identified one key mechanism that targets the host colonocytes to modulate microbiome composition and the resulting host-immune profile. Using upstream inflammasome targets like NOD-like receptor pyrin domain-3 (NLRP3) that are implicated as a causal mechanism for gut dysbiosis and microbiome enrichment and production of more pro-inflammatory metabolites, we used one of our lead molecules that had been identified as the first-in-class and best-in-class SB_NI_112, an NLRP3-NF-κβ (Nuclear factor kappa B) inflammasome inhibitor, to target colonocytes and modulate the gut microbiota. This lead molecule has shown excellent safety and biodistribution profile in pre-clinical studies.

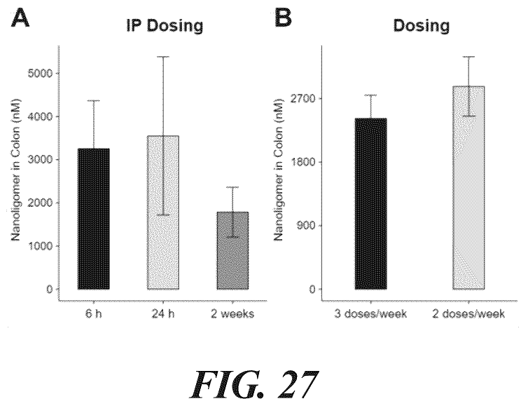

Still referring to FIGS. 14, 22, and 27, we conducted PK/PD Biodistribution and Safety Toxicology studies in mice. Although the Nanoligomers have shown remarkably low Kd values (2-18 nM) and excellent clearance of non-host targeting Nanoligomer molecules, Nanoligomers show high bioavailability profile for on-target engagement (FIG. 27). For example, using a single 500 mg/kg dose and tracking it over 2 weeks, we observed excellent concentration (˜3000 nM in the colon, and drug half-lifetime (˜28 days, FIG. 27A). Following dosing of lower maintenance doses of 150 mg/kg using 3 and 2 doses per week, we saw identical concentrations, and therefore are not limited by drug- availability (FIG. 27B). Further evaluation of safety, tox, and potential immunogenic response at a high concentration of 500 mg/kg dosing, showed no accumulation or immunogenic response, as monitored through serum 36-plex cytokine measurements as well as H&E stained histology images (FIG. 14, first pass and target organ colon). These datasets show favorable pharmacological properties using both intraperitoneal (IP) and oral dosing (enteric-coated capsules).

Now referring to FIG. 27, PK/PD safety and toxicology for nanoligomer therapeutic molecule data is provided. (A) A single intraperitoneal (IP) administration of 500 mg/kg nanoligomer dose tracked through time shows 28-day half life of the drug on target. (B) dosing 150 mg/kg nanoligomer 3 and 2 times per week shows identical drug profile in colon.

Now referring to FIGS. 28 and 29, a dextran sodium sulfate (DSS) model is provided and data is shown. First, the C57BL/6 mice were acclimatized and then treated with antibiotics for 5 days, to wipe out their microbiome, and then recolonized with fecal gavage prepared from 10 different IBD patients (collected at the University of Colorado Anschutz). We also spiked the fecal gavage with E. rectale, A. muciniphila, Blautia coccoides, and Alistipes shahii. Following one week of successful recolonization of the gut of these mice (both C57BL/6 for chemically-induced), with IBD relevant/representative microbiome. Following IBD microbiome colonization in a gnotobiotic mice-facility, for DSS-induced colitis, the mice were introduced to 3% dextran sodium sulfate (DSS) in drinking water for 5 days, and then replaced back with normal drinking water, for a total of 3 cycles. After colitis induction, the Nanoligomers (SB_BGC_CK1 and SB_NI_112, FIG. 28A) were administered using IP (FIG. 28) and oral routes (FIG. 29) 3 times/week during weeks when DSS water is used. The disease activity index (DAI) scores were recorded to monitor weights, stool consistency, and potential blood in the stool. Following the experiment, the mice were euthanized and their colons were harvested. The colons were used for 2 biochemical endpoint studies: 1) 36-panel cytokine panel analysis of colon tissue; and 2) histology.

Still referring to FIGS. 28 and 29, we assessed the two lead Nanoligomers in the mouse colitis models. As shown here, both lead molecules showed a rapid drop in the disease activity index (DAI) scores with just 1-week of treatment. Mice with sham (sterile saline or enteric-coated pills with filler alone) showed high disease severity, characterized by loose/watery stool and blood. However, the mice with treatment (IP and pills with Nanoligomer) showed complete reversal of disease in most mice within a week (FIG. 28B-F). On further biochemical assessment of colon tissue, homogenized using ThermoFisher homogenizing solution, and then evaluated using a 36-cytokine panel revealed a significant reduction in inflammation, correlating with DAI scores. Using TNF and IL-17 as two biomarkers of IBD inflammation and the targets of key disease-modifying therapies in the area such as Humira, Remicade, JAK-STAT inhibitors, etc., showed significant reduction (SB_NI_112 showed an average of 90% reduction in TNF and 85% reduction in IL-17, SB_BGC_CK1 showed 66% and 50% reduction in TNF and IL-17, respectively, FIG. 28C, D), within 1-week using IP route of administration. Further assessment during a 3-week chronic IBD study using oral route (FIG. 29) showed decrease in key inflammatory markers associated with IBD to be reduced in colons upon treatment with Nanoligomers.

Now referring to FIG. 28, information on a dextran sodium sulfate model is provided. (A) Schematic showing two nanoligomer molecules SB_BGC_CK1 and SB_NI_112 being assessed in a colitis mouse model. (B) Disease activity index (DAI) scores showing immediate relief from colitis in just 1 week of dosing (3 doses/week) for both lead molecules. (C) TNF-biomarker shows 90% reduction with NI_112 and 66% reduction with CK1 respectively in DSS-induced colitis. (D) using IL-17 as a biomarker, 85% reduction with NI_112 and 50% reduction in CK1 was observed. € slight increases in M-CSF and growth factors aid in colon healing. (F) representative histology images showing reduced inflammation with both lead nanoligomers. N=5 in mice, IP administration.

Now referring to FIG. 29, data from oral drug administration of SB_NI_112 shows effectiveness over a 3 week DSS study. Nanoligomers are administered 3 times per week at a dose of 75 mg/kg. Statistically significant reduction of expression of key inflammatory markers of IBD compared to vehicle controls are observed in mouse colons: (A-D) TNF-alpha (A), IL-23 (B), IL-17 (C), IL-12p70 (D), while IFN-alpha (E) expression remains unchanged. N=5 mice, oral route (75 mg/kg API), placebo: filler in capsules with no API, 3-week chronic DSS-induced IBD.

Now referring to FIG. 30, data on a TNF^{ΔARE/+} model is provided. This model involves a mutation in the adenylate-uridylate-rich element (ARE) of the TNF gene, leading to increased stability of TNF mRNA and higher TNF production. Elevated TNF levels contribute to intestinal inflammation and colitis in these mice. TNF^{ΔARE} model is a standard model of human Chron's disease and is characterized by elevated TNF expression and inflammation in colon as is observed in Chron's disease patients. Similar to DSS model, the TNF^{ΔARE/+} mice were acclimatized for 2 weeks, and then treated with antibiotics for 5 days, to wipe out their microbiome, and then recolonized with fecal gavage prepared from 10 different IBD patients (collected at the University of Colorado Anschutz). We also spiked the fecal gavage with E. rectale, A. muciniphila, Blautia coccoides, and Alistipes shahii between week 6-7. Following one week of successful recolonization of the gut of these TNF^{ΔARE/+} with IBD relevant/representative microbiome, the mice were divided into four groups: 1) TNF^{ΔARE/+}+Saline (positive control), 2) TNF^{ΔARE/+}SB_NI_112, 3) TNF^{ΔARE/+}+SB_BGC_CK1, and 4) WT (negative control). Post 4-weeks of treatment with 3 doses/week of 150 mg/kg administered IP, mouse was euthanized and colon tissue samples were harvested for cytokine analysis. Evaluation of cytokines continued to validate the efficacy of both lead molecules as shown by the significant reduction of key IBD associated inflammatory cytokines including TNF-alpha, IL-23, IL-17A, IL-12p70 (FIG. 30), and lack of change in IFN-pathways, again re-affirming that the lead molecules do not suppress the ability of fight infections. While TNF^{ΔARE/+} mice show lower inflammation compared to DSS-induction, the biomarkers were reduced to their respective negative controls. These results provide strong evidence for the efficacy of lead molecules, and therefore in this proposal we pursue further translation of these molecules to the clinic.

Still referring to FIG. 30, IP administration of SB_NI_112 shows effectiveness in a genetic TNF^{ΔARE} model. Nanoligomers were administered 3 times per week at 150 mg/kg. Statistically significant reduction of expression of key inflammatory markers of IBD compared to vehicle controls are observed in mouse colons: TNF-alpha (A), IL-23 (B), IL-17 (C), IL-12p70 (D). N>=5 mice per group. * indicates p<0.05.

Now referring to FIG. 31, a preliminary safety and PK/PD study in a large animal model was conducted. We performed preliminary PK/PD study in canine model, where we translated the dose of 75 mg/kg oral dose in mice to 33 mg/kg dose in dog using established formula in literature. An exhaustive blood work analysis shows no adverse effect observed in the animals over a period of two weeks. A representative set of metabolic and immune markers are shown in FIG. 31. Importantly, no statistical changes in immune cell distribution were found with percentages of immune cells populations within healthy range. This data further highlights that SB_NI_112 does not suppress the body's ability to fight infections.

Still referring to FIG. 31, data on a preliminary safety and PK/PD study in a canine (beagle) model are provided. SB_NI_112 was administered orally at a dose of 33 mg/kg (two capsules). No statistical difference was observed over a period of 2 weeks as shown by representative markers including levels of liver enzyme ALP (A), albumin (b). The immune cell distribution was not affected, shown here by neutrophils (C) and Lymphocytes (D), providing evidence that SB_NI_112 does not suppress the body's ability to fight infections.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

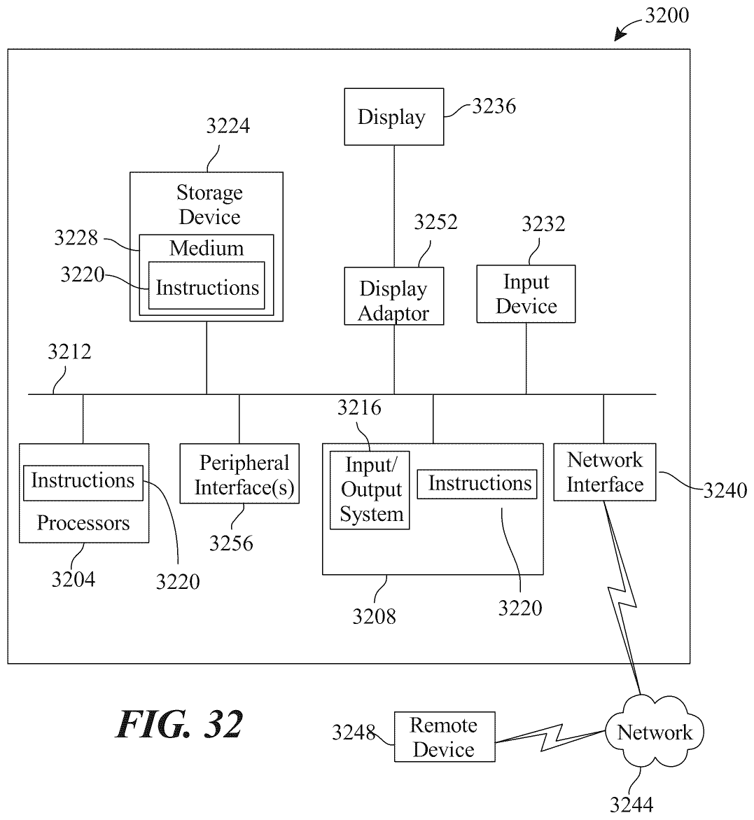

FIG. 32 shows a diagrammatic representation of one embodiment of a computing device in the exemplary form of a computer system 3200 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 3200 includes a processor 3204 and a memory 3208 that communicate with each other, and with other components, via a bus 3212. Bus 3212 may include any of several types of bus structures including, but not limited to, a memory bus, a memory controller, a peripheral bus, a local bus, and any combinations thereof, using any of a variety of bus architectures.

Processor 3204 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 3204 may be organized according to Von Neumann and/or Harvard architecture as a non-limiting example. Processor 3204 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 3208 may include various components (e.g., machine-readable media) including, but not limited to, a random-access memory component, a read only component, and any combinations thereof. In one example, a basic input/output system 3216 (BIOS), including basic routines that help to transfer information between elements within computer system 3200, such as during start-up, may be stored in memory 3208. Memory 3208 may also include (e.g., stored on one or more machine-readable media) instructions (e.g., software) 3220 embodying any one or more of the aspects and/or methodologies of the present disclosure. In another example, memory 3208 may further include any number of program modules including, but not limited to, an operating system, one or more application programs, other program modules, program data, and any combinations thereof.

Computer system 3200 may also include a storage device 3224. Examples of a storage device (e.g., storage device 3224) include, but are not limited to, a hard disk drive, a magnetic disk drive, an optical disc drive in combination with an optical medium, a solid-state memory device, and any combinations thereof. Storage device 3224 may be connected to bus 3212 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 3224 (or one or more components thereof) may be removably interfaced with computer system 3200 (e.g., via an external port connector (not shown)). Particularly, storage device 3224 and an associated machine-readable medium 3228 may provide nonvolatile and/or volatile storage of machine-readable instructions, data structures, program modules, and/or other data for computer system 3200. In one example, software 3220 may reside, completely or partially, within machine-readable medium 3228. In another example, software 3220 may reside, completely or partially, within processor 3204.

Computer system 3200 may also include an input device 3232. In one example, a user of computer system 3200 may enter commands and/or other information into computer system 3200 via input device 3232. Examples of an input device 3232 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 3232 may be interfaced to bus 3212 via any of a variety of interfaces (not shown) including, but not limited to, a serial interface, a parallel interface, a game port, a USB interface, a FIREWIRE interface, a direct interface to bus 3212, and any combinations thereof. Input device 3232 may include a touch screen interface that may be a part of or separate from display 3236, discussed further below. Input device 3232 may be utilized as a user selection device for selecting one or more graphical representations in a graphical interface as described above.

A user may also input commands and/or other information to computer system 3200 via storage device 3224 (e.g., a removable disk drive, a flash drive, etc.) and/or network interface device 3240. A network interface device, such as network interface device 3240, may be utilized for connecting computer system 3200 to one or more of a variety of networks, such as network 3244, and one or more remote devices 3248 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 3244, may employ a wired and/or a wireless mode of communication. In general, any network topology may be used. Information (e.g., data, software 3220, etc.) may be communicated to and/or from computer system 3200 via network interface device 3240.

Computer system 3200 may further include a video display adapter 3252 for communicating a displayable image to a display device, such as display device 3236. Examples of a display device include, but are not limited to, a liquid crystal display (LCD), a cathode ray tube (CRT), a plasma display, a light emitting diode (LED) display, and any combinations thereof. Display adapter 3252 and display device 3236 may be utilized in combination with processor 3204 to provide graphical representations of aspects of the present disclosure. In addition to a display device, computer system 3200 may include one or more other peripheral output devices including, but not limited to, an audio speaker, a printer, and any combinations thereof. Such peripheral output devices may be connected to bus 3212 via a peripheral interface 3256. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve systems and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A nanoligomer comprising:
a targeting sequence, wherein the targeting sequence comprises a polynucleotide binding domain and a transcription activation domain; and
a nanostructure, wherein the nanostructure comprises a cell uptake domain.

2. The nanoligomer of claim 1, wherein the polynucleotide binding domain is capable of hybridizing with a polynucleotide encoding NLRP3.

3. The nanoligomer of claim 1, wherein the polynucleotide binding domain is capable of hybridizing with a polynucleotide encoding NF-κβ.

4. The nanoligomer of claim 1, wherein the transcription activation domain comprises an acidic domain.

5. The nanoligomer of claim 4, wherein the targeting sequence is capable of hybridizing to a promoter of a polynucleotide sequence that has an associated TATA box.

6. The nanoligomer of claim 1, wherein the transcription activation domain comprises a glutamine rich domain.

7. The nanoligomer of claim 6, wherein the targeting sequence is capable of hybridizing to a promoter of a polynucleotide sequence that has an associated GC box.

8. The nanoligomer of claim 1, wherein the transcription activation domain comprises a proline rich domain.

9. The nanoligomer of claim 8, wherein the targeting sequence is capable of hybridizing to a promoter of a polynucleotide sequence that has an associated CCAAT box.

10. The nanoligomer of claim 1, wherein the transcription activation domain comprises an isoleucine rich domain.

11. The nanoligomer of claim 1, wherein the transcription activation domain comprises a sequence selected from the group consisting of SEQ ID NO: 30-45.

12. The nanoligomer of claim 1, wherein the hydrodynamic size of the nanoligomer is no more than 5 nm.

13. The nanoligomer of claim 1, wherein the nanoligomer is a brain penetrating nanoligomer and has a hydrodynamic size of less than 2 nm.

14. The nanoligomer of claim 1, wherein the nanoligomer is a peripherally restricted nanoligomer and has a hydrodynamic size of 4 nm to 5 nm.

15. The nanoligomer of claim 1, wherein the nanoligomer does not form a protein corona in serum.

16. The nanoligomer of claim 1, wherein the cell uptake domain has a length of between 1 and 3 amino acids.

17. The nanoligomer of claim 1, wherein the targeting sequence further comprises a linker.

18. The nanoligomer of claim 1, wherein the targeting sequence further comprises a nanostructure binding domain.

19. The nanoligomer of claim 1, further comprising a nuclear localization sequence.

20. The nanoligomer of claim 1, wherein the nanostructure comprises a transition metal nanoparticle.
